(19) United States
(12) Patent Application Publication
(10) Pub. No.: US 2024/0328947 A1
(43) Pub. Date: Oct. 3, 2024

(54) SYSTEMS AND METHODS FOR IMPROVING PARTICLE PROCESSING

(71) Applicant: Ultima Genomics, Inc., Fremont, CA (US)

(72) Inventors: Robert ONO, Palo Alto, CA (US); Aklilu WORKU, San Francisco, CA (US); Stephanie KUBECKA, San Jose, CA (US); Florian OBERSTRASS, Redwood City, CA (US); Gilad ALMOGY, Palo Alto, CA (US)

(21) Appl. No.: 18/635,829

(22) Filed: Apr. 15, 2024

Related U.S. Application Data
(63) Continuation of application No. PCT/US2022/047318, filed on Oct. 20, 2022.
(60) Provisional application No. 63/270,432, filed on Oct. 21, 2021.
(60) Provisional application No. 63/315,058, filed on Feb. 28, 2022.

(51) Int. Cl. G01N 21/64; C12Q 1/6806; C12Q 1/6834

(52) U.S. Cl. CPC G01N 21/6456; C12Q 1/6806; C12Q 1/6834

(57) ABSTRACT

Disclosed herein are buffer solutions for decreasing the size of particles during imaging. The buffer solutions may comprise polymers, ions, or a combination thereof. The buffer solutions may decrease the sizes of particles to facilitate the resolution of individual particles during imaging. Also provided herein are methods and systems of using the same.

### CROSS-REFERENCE

This application is a continuation of International Patent Application No. PCT/US2022/047318, filed Oct. 20, 2022, which claims the benefit of U.S. Provisional Patent App. No. 63/270,432, filed Oct. 21, 2021, and U.S. Provisional Patent App. No. 63/315,058, filed Feb. 28, 2022, each of which is entirely incorporated herein by reference.

### BACKGROUND

Biological sample processing, such as the detection, quantification, and/or sequencing of cells and biological molecules, has various applications in the fields of molecular biology and medicine (e.g., diagnosis). Genetic testing may be useful for a number of diagnostic methods. For example, disorders that are caused by rare genetic alterations (e.g., sequence variants) or changes in epigenetic markers, such as cancer and partial or complete aneuploidy, may be detected or more accurately characterized with deoxyribonucleic acid (DNA) or ribonucleic acid (RNA) sequence information. For example, nucleic acid sequencing can be used to provide sequence information for a nucleic acid sample. Such sequence information may be used to diagnose a certain condition in a subject and in some cases tailor a treatment plan. As another example, research into pathogens may lead to treatment of contagious diseases. Sequencing is widely used for molecular biology applications, including vector designs, gene therapy, vaccine design, industrial strain design and verification.

Biological sample processing may involve a fluidics system and/or a detection system. In some cases, nucleic acid molecules may be sequenced while coupled to particles such as beads. However, the use of such particle-based approaches may reduce sequencing quality via, for example, aggregation of particles via associations between nucleic acid molecules bound to the particles. Thus, despite the advance of sequencing technology, determining sequences with spatial resolution still requires laborious efforts.

### SUMMARY

Disclosed herein are methods, systems, and compositions for particle processing that improve sequencing quality. The methods, systems, and compositions may modulate the size of particles during imaging. Increased particle sizes may cause the signals of two particles imaged to overlap. The overlapped signal may interfere with the detection and resolution of the two particles during imaging. Decreasing the sizes of the particles during imaging can improve the resolution of individual particle imaged. In some cases, decreasing the sizes of the particles can improve the resolution of individual reads of sequencing. The methods, systems, and compositions may reduce particle aggregation and/or increase substrate loading efficiency. Decreasing the average sizes of particles during loading can increase the number of particles that can be loaded onto a substrate for sequencing. Particles processed according to the methods, systems, and compositions provided herein may be useful in nucleic acid sequencing applications.

Provided herein, are methods for processing a substrate. In an aspect, a method for processing a substrate comprises: (a) providing the substrate comprising a plurality of individually addressable locations, wherein a plurality of particles is immobilized to at least a subset of the plurality of individually addressable locations; (b) adding a buffer solution to the substrate, wherein the buffer solution decreases an average size of at least a subset of the plurality of particles; and (c) subsequent to (b), imaging at least a portion of the substrate.

In another aspect, a method for processing a substrate comprises: (a) providing the substrate comprising a plurality of individually addressable locations, wherein a plurality of particles is immobilized to at least a subset of the plurality of individually addressable locations; and (b) adding a buffer solution comprising polyethylene glycol (PEG) to the substrate, wherein the buffer solution decreases an average size of at least a subset of the plurality of particles.

In another aspect, a method for processing a substrate comprises: (a) providing the substrate comprising a plurality of individually addressable locations, wherein a plurality of particles is immobilized to at least a subset of the plurality of individually addressable locations; and (b) adding a buffer solution, across an air gap, to the substrate, wherein the buffer solution decreases an average size of at least a subset of the plurality of particles.

In another aspect, a method for processing a substrate comprises: (a) providing the substrate comprising a plurality of individually addressable locations, wherein the plurality of individually addressable locations is substantially planar, wherein a plurality of particles is immobilized to at least a subset of the plurality of individually addressable locations; and (b) adding a buffer solution to the substrate, wherein the buffer solution decreases an average size of at least a subset of the plurality of particles.

In another aspect, a method for processing a substrate comprises: (a) providing the substrate comprising a plurality of individually addressable locations, wherein the plurality of individually addressable locations are protrusions from a base surface of the substrate, wherein a plurality of particles is immobilized to at least a subset of the plurality of individually addressable locations; and (b) adding a buffer solution to the substrate, wherein the buffer solution decreases an average size of at least a subset of the plurality of particles.

In another aspect, a method for processing a substrate comprises: (a) providing the substrate comprising a plurality of individually addressable locations, wherein a plurality of particles is immobilized to at least a subset of the plurality of individually addressable locations; and (b) adding a buffer solution comprising spermine or a derivative thereof to the substrate, wherein the buffer solution decreases an average size of at least a subset of the plurality of particles.

In another aspect, a method for processing a substrate comprises: (a) providing the substrate comprising a plurality of individually addressable locations, wherein a plurality of particles is immobilized to at least a subset of the plurality of individually addressable locations; (b) adding a buffer solution to the substrate, wherein the buffer solution decreases an average size of at least a subset of the plurality of particles; and (c) imaging at least a portion of the substrate, wherein the buffer solution is present on the substrate during the imaging.

In another aspect, a method for processing a substrate comprises: (a) providing the substrate comprising a plurality of individually addressable locations, wherein a plurality of particles is electrostatically immobilized to at least a subset of the plurality of individually addressable locations; and (b) adding a buffer solution to the substrate, wherein the buffer solution decreases an average size of at least a subset of the plurality of particles.

In some embodiments, the imaging detects at least 1000 particles of the plurality of particles. In some embodiments, the imaging detects at least 10,000 particles of the plurality of particles. In some embodiments, the imaging detects at least 100,000 particles of the plurality of particles. In some embodiments, the imaging detects at least 1,000,000,000 particles of the plurality of particles. In some embodiments, the imaging detects at least 10% of the plurality of particles. In some embodiments, the imaging detects at least 50% of the plurality of particles. In some embodiments, the imaging detects at least 70% of the plurality of particles. In some embodiments, the imaging detects at least 90% of the plurality of particles. In some embodiments, the imaging comprises using photometry.

In some embodiments, the method further comprises, subsequent to the imaging of (c), washing the substrate of the buffer solution. In some embodiments, upon or subsequent to the washing, the average size of the at least the subset of the plurality of particles increases. In some embodiments, upon or subsequent to the washing, the average size of the at least the subset of the plurality of particles returns to within 10% of the average size of the plurality of particles in (a). In some embodiments, upon or subsequent to the washing, the average size of the at least the subset of the plurality of particles returns to within 5% of the average size of the plurality of particles in (a).

In some embodiments, the method further comprises, subsequent to the washing, (i) performing one or more operations on or with the plurality of particles on the substrate, (ii) repeating (b), and (iii) repeating the imaging. In some embodiments, the method further comprises repeating a cycle of (i)-(iii) at least 20 times, with washing in between each cycle. In some embodiments, the one or more operations comprise a nucleotide incorporation reaction with a nucleic acid molecule immobilized to a particle of the plurality of particles.

In some embodiments, the method further comprises, subsequent to adding the buffer solution of (b), (c) imaging at least a portion of the substrate. In some embodiments, the imaging detects at least 1000 particles of the plurality of particles. In some embodiments, the imaging detects at least 10,000 particles of the plurality of particles. In some embodiments, the imaging detects at least 100,000 particles of the plurality of particles. In some embodiments, the imaging detects at least 1,000,000,000 particles of the plurality of particles. In some embodiments, the imaging detects at least 10% of the plurality of particles. In some embodiments, the imaging detects at least 50% of the plurality of particles. In some embodiments, the imaging detects at least 70% of the plurality of particles. In some embodiments, the imaging detects at least 90% of the plurality of particles. In some embodiments, the method further comprises subsequent to the imaging of (c), washing the substrate of the buffer solution. In some embodiments, upon or subsequent to the washing, the average size of the at least the subset of the plurality of particles increases. In some embodiments, upon or subsequent to the washing, the average size of the at least the subset of the plurality of particles returns to within 10% of the average size of the plurality of particles in (a). In some embodiments, upon or subsequent to the washing, the average size of the at least the subset of the plurality of particles returns to within 5% of the average size of the plurality of particles in (a). In some embodiments, the method further comprises subsequent to the washing, (i) performing one or more operations on or with the plurality of particles on the substrate, (ii) repeating (b), and (iii) repeating the imaging. In some embodiments, the method further comprises repeating a cycle of (i)-(iii) at least 20 times, with washing in between each cycle. In some embodiments, the one or more operations comprise a nucleotide incorporation reaction with a nucleic acid molecule immobilized to a particle of the plurality of particles. In some embodiments, the imaging comprises using photometry.

In some embodiments, the buffer solution comprises Tris. In some embodiments, the Tris is the buffer solution has a concentration of 20 mM. In some embodiments, the buffer solution comprises NaCl. In some embodiments, the NaCl in the buffer solution has a concentration of about 10 μM. In some embodiments, the NaCl in the buffer solution has a concentration of about 80 μM. In some embodiments, the NaCl in the buffer solution has a concentration of about 800 μM. In some embodiments, the NaCl in the buffer solution has a concentration of about 8 mM. In some embodiments, the NaCl in the buffer solution has a concentration of about 80 mM.

In some embodiments, the average size, as measured in full-width-half-maximum (FWHM), of the plurality of particles prior to (b) is about 0.1 μm. In some embodiments, the average size, as measured in full-width-half-maximum (FWHM), of the plurality of particles prior to (b) is about 1 μm. In some embodiments, the decrease of the average size in (b) is at least about 1%. In some embodiments, the decrease of the average size in (b) is at least about 10%. In some embodiments, the decrease of the average size in (b) is at least about 25%.

In some embodiments, the plurality of particles comprises a plurality of beads. In some embodiments, a bead of the plurality of beads comprises a nucleic acid molecule immobilized thereto. In some embodiments, the nucleic acid molecule comprises a fluorescent dye. In some embodiments, the bead comprises a plurality of nucleic acid molecules, including the nucleic acid molecule, immobilized thereto. In some embodiments, nucleic acid molecules of the plurality of nucleic acid molecules have sequence homology with each other.

In some embodiments, the PEG has an average molecular weight of at least 100 daltons. In some embodiments, the PEG has an average molecular weight of at least 4000 daltons. In some embodiments, the PEG has an average molecular weight of at least 8000 daltons. In some embodiments, the PEG is at least 0.1%, by weight, in the buffer solution. In some embodiments, the PEG is at least 1%, by weight, in the buffer solution. In some embodiments, the PEG is at least 5%, by weight, in the buffer solution. In some embodiments, the PEG is at least 10%, by weight, in the buffer solution.

In some embodiments, the buffer solution comprises an ion or a salt derivative thereof. In some embodiments, the ion comprises a cation or a salt derivative thereof. In some embodiments, the cation comprises a divalent cation. In some embodiments, the divalent cation comprises a magnesium ion. In some embodiments, the magnesium ion comprises Mg²⁺. In some embodiments, the salt comprises a chloride salt. In some embodiments, the ion or the salt derivative in the buffer solution has a concentration of about 1 μM. In some embodiments, the ion or the salt derivative in the buffer solution has a concentration of about 50 μM. In some embodiments, the ion or the salt derivative in the buffer solution has a concentration of about 500 μM. In some embodiments, the ion or the salt derivative in the buffer solution has a concentration of about 5 mM. In some embodiments, the ion or the salt derivative in the buffer solution has a concentration of about 50 mM.

In some embodiments, the spermine or the derivative thereof in the buffer solution has a concentration of about 1 μM. In some embodiments, the spermine or the derivative thereof in the buffer solution has a concentration of about 50 μM. In some embodiments, the spermine or the derivative thereof in the buffer solution has a concentration of about 500 μM. In some embodiments, the spermine or the derivative thereof in the buffer solution has a concentration of about 5 mM. In some embodiments, the spermine or the derivative thereof in the buffer solution has a concentration of about 10 mM.

In another aspect, provided is a method for processing a substrate, comprising: (a) dispensing a first plurality of particles in a first buffer solution onto the substrate, wherein a subset of the first plurality of particles immobilizes onto a first plurality of individually addressable locations on the substrate; (b) adding a second buffer solution to the substrate, wherein an average maximum dimension of the subset of the first plurality of particles decreases upon contacting the second buffer solution; and (c) dispensing a second plurality of particles in the first buffer solution onto the substrate, wherein a subset of the second plurality of particles immobilizes onto a second plurality of individually addressable locations on the substrate.

In some embodiments, subsequent to (c), the method further comprises imaging (d) at least a portion of the substrate. In some embodiments, the imaging comprises using photometry.

In some embodiments, a particle in the subset of the first plurality of particles comprises a nucleic acid molecule immobilized thereto. In some embodiments, the nucleic acid molecule comprises a fluorescent dye. In some embodiments, the particle comprises a plurality of nucleic acid molecules, including the nucleic acid molecule, immobilized thereto. In some embodiments, nucleic acid molecules of the plurality of nucleic acid molecules have sequence homology with each other. In some embodiments, a particle in the subset of the second plurality of particles comprises a nucleic acid immobilized thereto.

In some embodiments, the adding (b) further comprises making one or more individually addressable locations in the first plurality of individually addressable locations available for immobilizing particles. In some embodiments, prior to the dispensing (c), the second plurality of individually addressable locations do not have a particle of the first plurality of particles immobilized thereto.

In some embodiments, the method further comprises prior to the imaging (d), washing the substrate of the first buffer solution.

In some embodiments, upon or subsequent to the dispensing (c), the average maximum dimension of the subset of the first plurality of particles increases.

In some embodiments, the first plurality of individually addressable locations comprises at least about 100,000 locations. In some embodiments, the second plurality of individually addressable locations comprises at least about 100,000 locations.

In some embodiments, the imaging (d) detects 1000-10,000 particles immobilized to individually addressable locations on the substrate.

In some embodiments, the average maximum dimension, as measured in full-width half-maximum (FWHM), of the subset of the first plurality of particles prior to the adding (b) is about 0.1 μm. In some embodiments, the average maximum dimension, as measured in full-width half-maximum (FWHM), of the subset of the first plurality of particles prior to the adding (b) is about 1 μm. In some embodiments, the average maximum dimension of the subset of the first plurality of particles upon the adding (b) decreases by at least about 1%. In some embodiments, the average maximum dimension of the subset of the first plurality of particles upon the adding (b) decreases by at least about 10%. In some embodiments, the average maximum dimension of the subset of the first plurality of particles upon the adding (b) decreases by at least about 25%.

In some embodiments, the first plurality of particles comprises a first plurality of beads and the second plurality of particles comprises a second plurality of beads.

In some embodiments, the adding (b) comprises adding the second buffer solution across an air gap to the substrate.

In some embodiments, the second buffer solution comprises polyethylene glycol (PEG). In some embodiments, the PEG has an average molecular weight of at least 100 daltons. In some embodiments, the PEG has an average molecular weight of at least 4000 daltons. In some embodiments, the PEG has an average molecular weight of at least 8000 daltons. In some embodiments, the second buffer solution comprises at least 0.1% by weight of PEG. In some embodiments, the second buffer solution comprises at least 1% by weight of PEG. In some embodiments, the second buffer solution comprises at least 5% by weight of PEG. In some embodiments, the second buffer solution comprises at least 10% by weight of PEG.

In some embodiments, the second buffer solution comprises a divalent cation. In some embodiments, the divalent cation comprises a magnesium ion or a calcium ion.

In some embodiments, the dispensing (a) comprises incubating the first plurality of particles in the first buffer solution on the substrate for a time period of at least 30 minutes; and the dispensing (b) comprises incubating the second plurality of particles in the second buffer solution on the substrate for a time period of at least 30 minutes. In some embodiments, the dispensing (a) comprises incubating the first plurality of particles in the first buffer solution on the substrate for a time period of at least 60 minutes; and the dispensing (b) comprises incubating the second plurality of particles in the second buffer solution on the substrate for a time period of at least 60 minutes.

In another aspect, provided is a method of processing a substrate, comprising: (a) providing a plurality of particles in a first buffer solution; (b) adding an aliquot of a second buffer solution to the plurality of particles in the first buffer solution, thereby providing the plurality of particles in a third buffer solution; (c) incubating the plurality of particles with the third buffer solution; and (d) dispensing the plurality of particles onto the substrate to immobilize at least a subset of the plurality of particles onto a plurality of individually addressable locations on the substrate.

In some embodiments, the method further comprises, subsequent to the dispensing (d), imaging at least a portion of the substrate. In some embodiments, the imaging comprises using photometry. In some embodiments, the method further comprises, prior to the imaging (d), washing the substrate of the third buffer solution.

In some embodiments, a particle in the at least the subset of the plurality of particles comprises a nucleic acid molecule immobilized thereto. In some embodiments, the nucleic acid molecule comprises a fluorescent dye. In some embodiments, the particle comprises a plurality of nucleic acid molecules, including the nucleic acid molecule, immobilized thereto. In some embodiments, nucleic acid molecules of the plurality of nucleic acid molecules have sequence homology with each other.

In some embodiments, the second buffer solution comprises at least 1% by weight of PEG. In some embodiments, the second buffer solution comprises at least 5% by weight of PEG.

In some embodiments, the second buffer solution comprises a divalent cation. In some embodiments, the divalent cation is a magnesium ion or a calcium ion.

In some embodiments, the third buffer solution comprises at least 0.1% by weight of PEG. In some embodiments, the third buffer solution comprises at least 1% by weight of PEG.

In some embodiments, first buffer solution does not comprise PEG.

In some embodiments, the first buffer solution does not comprise a divalent cation.

In some embodiments, the first buffer solution or the second buffer solution comprises a Tris buffer solution.

In some embodiments, the plurality of individually addressable locations comprises at least about 100,000 locations.

In some embodiments, an average maximum dimension of the plurality of particles decreases upon or subsequent to the incubating (c). In some embodiments, the average maximum dimension, as measured in full-width half-maximum (FWHM), of the plurality of particles prior to the adding (b) is about 0.1 μm. In some embodiments, the average dimension, as measured in full-width half-maximum (FWHM), of the subset of the plurality of particles prior to the adding (b) is about 1 μm.

In some embodiments, the average dimension of the plurality of particles upon or subsequent to the incubating (c) decreases by at least about 1%. In some embodiments, the average dimension of the plurality of particles upon or subsequent to the incubating (c) decreases by at least about 10%. In some embodiments, the average dimension of the plurality of particles upon or subsequent to the incubating (c) decreases by at least about 25%.

In some embodiments, the incubating (c) comprises incubating the plurality of particles in the third buffer solution for a time period of at least 10 minutes. In some embodiments, the incubating (c) comprises incubating the plurality of particles in the third buffer solution for a time period of at least 1 hour.

In some embodiments, the plurality of particles comprises a plurality of beads.

In some embodiments, prior to the adding (b), the plurality of particles is stored in the first buffer solution for a time period of at least 1 hour. In some embodiments, prior to the adding (b), the plurality of particles is stored in the first buffer solution for a time period of at least 1 day.

Another aspect of the present disclosure provides a system comprising one or more computer processors and computer memory coupled thereto. The computer memory comprises machine executable code that, upon execution by the one or more computer processors, implements any of the methods above or elsewhere herein.

Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIGS. 1A-1G illustrate examples of cross-sectional surface profiles of a substrate, as described herein.

FIGS. 2A-2B illustrate methods for loading beads onto a substrate, as described herein. FIG. 2A illustrates a method for loading beads onto specific regions of a substrate. FIG. 2B illustrates a method for loading a subset of beads onto specific regions of a substrate.

FIG. 3 shows an example coating of a substrate with a hexagonal lattice of beads, as described herein.

FIGS. 4A-4B illustrate example systems and methods for loading a sample or a reagent onto a substrate, as described herein.

FIG. 5 shows a flowchart for an example method for sequencing a nucleic acid molecule, as described herein.

FIG. 6 illustrates a system with different environmental conditions in an open substrate system, as described herein.

FIGS. 7A-7C illustrate multiplexed stations in a sequencing system.

FIG. 8 shows a cross-sectional view of an example immersion optical system, as described herein.

FIG. 9 shows an example computerized system for sequencing a nucleic acid molecule, as described herein.

FIGS. 10A-10C shows non-limiting example images of a substrate loaded with particles (e.g., beads). The images depict beads in a control imaging buffer (FIG. 10A), beads in an imaging buffer that includes 10% PEG-4000 (FIG. 10B), and beads in another imaging buffer that includes both 10% PEG-4000 and 50 mM MgCl₂ (FIG. 10C).

FIGS. 11A-11C show non-limiting example images of a substrate loaded with particles (e.g., beads). The images depict beads in the control imaging buffer (FIG. 11A), beads in an imaging buffer that includes 10% PEG-8000 (FIG. 11B), and beads in another imaging buffer that includes 10% PEG-8000 and 50 mM MgCl₂ (FIG. 11C).

FIG. 12 shows non-limiting examples of the effect of PEG-4000 on bead size. The graph illustrates exemplary measurements of bead diameter for beads loaded on a substrate and imaged in the presence of imaging buffers, where each buffer includes a titrated percentage of PEG-4000 (the x-axis). Each buffer further includes 50 mM of MgCl₂. The diameter of the beads was determined as relative full width at half maximum (FWHM) of the beads (the y-axis), where bead diameters were normalized to beads in an imaging buffer with no PEG-4000.

FIG. 13 shows a non-limiting example of the effect of MgCl₂ on bead size. The graph illustrates exemplary measurements of bead diameter (represented as FWHM of the beads (y-axis)) for beads loaded on a substrate and imaged in the presence of imaging buffers, where each buffer includes a titrated amount of MgCl₂ (the x-axis). Each buffer further includes 5% PEG-4000.

FIGS. 14A and 14B show non-limiting examples of the FWHM of the beads loaded on a substrate and imaged under various conditions in an imaging buffer (20 mM Tris, 80 mM NaCl. TritonX) versus a shrinkage buffer (the imaging buffer+10% w/v PEG-4000 and 50 mM MgCl₂).

FIG. 15 shows the average relative FWHM of the beads (y-axis) loaded on a substrate imaged in the presence of a shrinkage buffer (20 mM Tris, 80 mM NaCl. TritonX with various amounts of spermine) (x-axis).

FIGS. 16A-16C illustrate non-limiting examples of a substrate (e.g., wafer) loaded with particles (e.g., beads), as described herein. The images depict beads in a first location on a wafer in the first imaging buffer (FIG. 16A), beads in the first location on the wafer in fifth imaging buffer that includes 10% PEG-8000 and 50 mM MgCl₂ (FIG. 16B), and beads in the first location on the wafer in the first imaging buffer after washing out the fifth imaging buffer (FIG. 16C).

FIG. 17 illustrates a non-limiting example of the average FWHM of beads (y-axis) loaded on a substrate and imaged under various buffers and conditions over multiple cycles.

FIG. 18 illustrates examples of patterns of individually addressable locations distributed on substrates, as described herein.

FIGS. 19A-19C illustrate representative, non-limiting examples of beads loaded onto wafer coupons. FIG. 19A illustrates the results of an initial loading of beads on a wafer coupon. FIG. 19B illustrates the same wafer coupon that has been incubated in a shrinking buffer (e.g., comprising 10% w/v PEG-8000), FIG. 19C illustrates the same wafer coupon after a second bead loading.

FIGS. 20A-20C show representative, non-limiting images of beads loaded onto wafer coupons with a pitch of 1.4 μm. FIG. 20A illustrates the results of loading beads that were suspended in a control buffer. FIG. 20B illustrates the results of loading beads in a first concentration of shrinking buffer (e.g., beads pre-shrunk in a shrinking buffer comprising PEG-8000). FIG. 20C illustrates the results of loading beads in a second concentration of shrinking buffer (e.g., beads pre-shrunk in a shrinking buffer comprising PEG-8000).

FIGS. 21A-21B show representative, non-limiting images of beads loaded onto wafer coupons. FIG. 21A illustrates beads on a wafer coupon imaged in the presence of buffer lacking PEG. FIG. 21B illustrates beads on the same wafer coupon imaged in the presence of a buffer comprising 1% w/v PEG-8000 and 50 nM MgCl₂.

FIGS. 22A-22D show representative images of bead aggregation on a wafer coupon upon loading onto the wafer after the beads were incubated for different amounts of time. FIG. 22A illustrates the results of loading the beads to the wafer immediately after incubation with TTM buffer. FIGS. 22B, 22C, and 22D illustrate the result of bead loading after 30 minutes, 60 minutes, or 120 minutes, respectively, of incubation of the beads with TTM buffer. Circles highlight selected bead aggregates that were detected. * indicates selected regions with significant aggregation. ** indicates selected regions of poor bead loading.

FIGS. 23A-23E show representative images of bead aggregation on a wafer coupon with a pitch size of 1.8 μm. The wafer surfaces were incubated in a buffer with different levels of Mg²⁺ (prewet) and the beads were incubated in a buffer with different levels of Mg²⁺ (load) before the beads were loaded onto the wafer surface. FIG. 23A illustrates a non-limiting example of bead loading where the substrate was incubated in Mg²⁺ and the beads were loaded using a buffer comprising Mg²⁺ FIGS. 23B, 23C, 23D, and 23E illustrate non-limiting examples of bead loading wherein the beads were loading using buffers lacking Mg²⁺, and the substrates were incubated with 50 mM Mg²⁺, 100 mM Mg²⁺, 200 mM Mg²⁺, and 300 mM Mg²⁺, respectively, prior to bead loading. Circles highlight selected bead aggregates that were detected.

FIGS. 24A-24B show representative images of bead aggregation on a wafer coupon with a pitch size of 1.5 μm. The wafer surfaces were incubated in a buffer with different levels of Mg²⁺ (prewet) and the beads were incubated in a buffer with different levels of Mg²⁺ (load) before the beads were loaded onto the wafer surface. Circles highlight example bead aggregates.

FIGS. 25A-25E show representative images of bead aggregation on a wafer coupon with a pitch size of 1×1.5 μm. The wafer surfaces were incubated in a buffer with different levels of Mg²⁺ (prewet) and the beads (amplified IH beads. B1434 (10%-tBA ATRP in THF). PA39FAM labeled) were incubated in a buffer with different levels of Mg²⁺ (load) before the beads were loaded onto the wafer surface. FIGS. 25A, 25B, 25C, 25D, and 25E illustrate non-limiting examples of bead loading wherein the wafer substrates were incubated with 0 mM Mg²⁺. 50 mM Mg²⁺, 100 mM Mg²⁺, 200 mM Mg²⁺, and 300 mM Mg²⁺, respectively. Circles highlight example bead aggregates that were detected.

### DETAILED DESCRIPTION

While various instances of the invention have been shown and described herein, it will be obvious to those skilled in the art that such instances are provided by way of example only. Numerous variations, changes, and substitutions may occur to those skilled in the art without departing from the invention. It should be understood that various alternatives to the instances of the invention described herein may be employed.

As used herein, the singular forms “a.” “an.” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc,), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40) bases, 50) bases, 100 bases, 200 bases, 300 bases, 400) bases, 500 bases, 1 kilobase (kb), 2 kb, 3 kb, 4 kb, 5 kb, 10 kb, 20 kb, 30 kb, 40 kb, 50 kb, 100 kb, 200 kb, 300 kb. 400 kb, 500 kb, 1 megabase (Mb), 10 Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxy hydroxylmethyl) uracil, 5-carboxy methylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxy carboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The terms “amplifying.” “amplification.” and “nucleic acid amplification” are used interchangeably and generally refer to generating one or more copies of a nucleic acid or a template. For example. “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor. S. et al. C.C. PNAS. 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron. Method for Performing Amplification of Nucleic Acid with Two Primers Bound to a Single Solid Support. Mosaic Technologies, Inc. (Winter Hill, Mass.); Whitehead Institute for Biomedical Research. Cambridge, Mass., (1997); Adessi et al., Nucl. Acids Res. 28: E87 (2000); Pemov et al., Nucl. Acids Res. 33: e11 (2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem, 320:55-65 (2003), each of which is incorporated herein by reference), and clonal amplification on beads using emulsions (Dressman et al., Proc. Natl. Acad. Sci. USA 100: 8817-8822 (2003), which is incorporated herein by reference) or ligation to bead-based adapter libraries (Brenner et al., Nat. Biotechnol. 18:630-634 (2000): Brenner et al., Proc. Natl. Acad. Sci. USA 97:1665-1670 (2000)): Reinartz, et al., Brief Funct. Genomic Proteomic 1: 95-104 (2002), each of which is incorporated herein by reference). Amplification products from a nucleic acid may be identical or substantially identical. A nucleic acid colony resulting from amplification may have identical or substantially identical sequences.

The terms “dispense” and “disperse” may be used interchangeably herein. In some cases, dispensing may comprise dispersing and/or dispersing may comprise dispensing. Dispensing generally refers to distributing, depositing, providing, or supplying a reagent, solution, or other object, etc. Dispensing may comprise dispersing, which may generally refer to spreading.

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering. Rayleigh scattering. Raman scattering, surface-enhanced Raman scattering. Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD). Hybrid TDI, complementary metal oxide semiconductor (CMOS) pseudo TDI device, or TDI line-scan camera.

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or a via a non-covalent interaction. A coupling between a first object and a second object may comprise a labile moiety, such as a moiety comprising an ester, vicinal diol, phosphodiester, peptidic, glycosidic, sulfone. Diels-Alder, or similar linkage. The strength of a coupling between a first object and a second object may be indicated by a dissociation constant. Kd, that indicates the inclination of a coupled object comprising a first object and a second object to dissociate into the uncoupled first and second objects and may be expressed as a ratio of dissociated (e.g., uncoupled) objects to coupled objects.

The term “label,” as used herein, generally refers to a moiety or dye that is capable of coupling with a species, such as, for example a nucleotide analog. A label may be used as an optically detectable moiety during sequencing methods and/or imaging methods described herein. A label may include an affinity moiety. In some cases, a label may be a detectable label (e.g., an optically detectable label) that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. In some cases, the coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl) phosphine (TCEP), tris(hydroxypropyl)phosphine (THP) or enzymatically cleavable (e.g., via an esterase, lipase, peptidase or protease). In some cases, the label may be luminescent: that is, fluorescent or phosphorescent. For example, the label may be or comprise a fluorescent moiety (e.g., a dye). Dyes and labels may be incorporated into nucleic acid sequences. Dyes and labels may also be incorporated into or attached to linkers, such as linkers for linking one or more beads to one another. For example, labels such as fluorescent moieties may be linked to nucleotides or nucleotide analogs via a linker (e.g., as described herein). A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromators, gratings, beam splitters, and lenses may be used to direct light to or from a fluorescent dye. A fluorescent dye may emit light (e.g., fluoresce) in the visible region of the electromagnetic spectrum ((e.g., between about 430-770) nm). A fluorescent dye may be excited over a single wavelength or a range of wavelengths. A fluorescent dye may be excitable by light in the red region of the visible portion of the electromagnetic spectrum (about 625-740) nm) (e.g., have an excitation maximum in the red region of the visible portion of the electromagnetic spectrum). Alternatively or additionally, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 625-740 nm) (e.g., have an emission maximum in the red region of the visible portion of the electromagnetic spectrum). Alternatively or additionally, fluorescent dye may emit signal in the green region of the visible portion of the electromagnetic spectrum (about 500-565 nm) (e.g., have an emission maximum in the green region of the visible portion of the electromagnetic spectrum).

Labeled nucleotides may comprise a dye, fluorophore, or quantum dot, Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580), DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5-(or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q, 532, and 633, or other fluorophores and quenchers. In some cases, the label may be one with linkers. For instance, a label may have a disulfide linker attached to the label. Non-limiting examples of such labels include Cy5-azide, Cy-2-azide, Cy-3-azide. Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

The term “open substrate,” as used herein, generally refers to a substrate (e.g., a surface for performing sequencing methods described herein) in which any point on an active surface of the substrate is physically accessible from a direction normal to the substrate. One or more surfaces of the substrate may be exposed to a surrounding open environment, and accessible from such surrounding open environment. For example, an array on the substrate may be exposed and accessible from such surrounding open environment. In some cases, as described elsewhere herein, the surrounding open environment may be controlled and/or confined in a larger controlled environment. Substrates that can be used in accordance with systems and methods described herein are described in further detail in U.S. Patent Pub. No. 2021/0079464, which is entirely incorporated herein by reference for all purposes.

### Sample Processing Systems

Despite the prevalence of sample processing systems and methods (e.g., nucleic acid sequencing systems and methods), such systems and methods may have low efficiency and can also be time-intensive. For example, sample processing systems and methods may not make maximal use of available surface area within the sample processing systems. Not making efficient use of analysis space inherently increases the throughput time for sample processing and correspondingly decreases the number of samples that can be processed overall. Thus, recognized herein is a need for methods and systems for sample processing and/or analysis with high efficiency.

Described herein are devices, systems, and methods that use open substrates or open flow cell geometries to process a sample. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in U.S. Patent Pub. Nos. 20200326327A1, 20210354126A1, and 20210079464A1, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

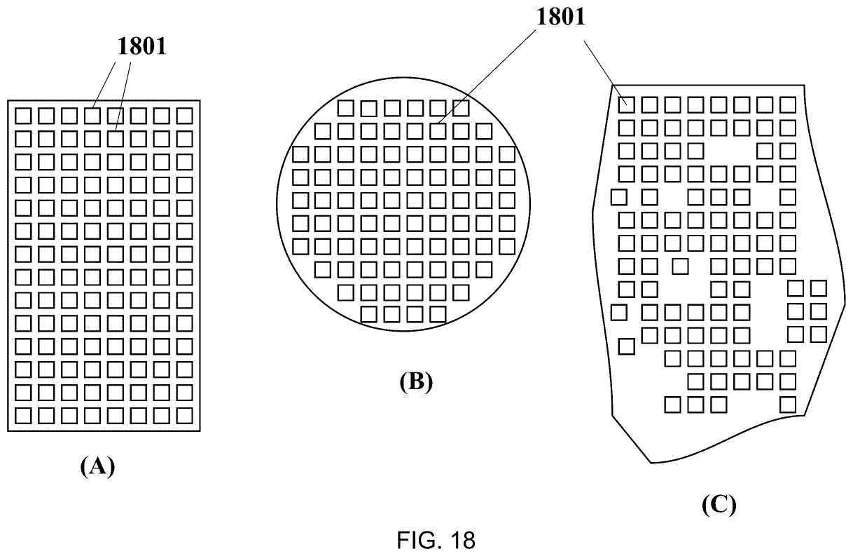

The plurality of individually addressable locations may be arranged as an array, randomly, or according to any pattern, on the substrate. FIG. 18 illustrates different substrates (from a top view) comprising different arrangements of individually addressable locations 1801, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries, as shown in FIG. 2A. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto), as shown in FIG. 2B. The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

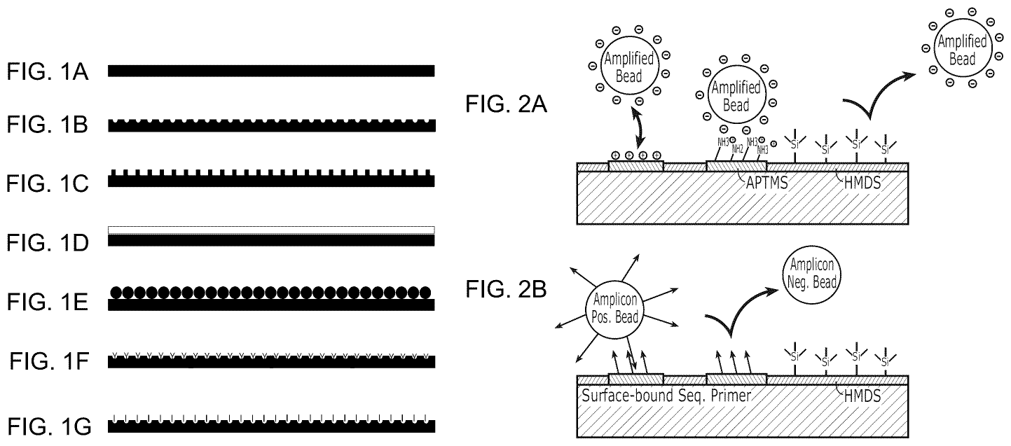

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 1A-1G illustrate different examples of cross-sectional surface profiles of a substrate. FIG. 1A illustrates a cross-sectional surface profile of a substrate having a completely planar surface. FIG. 1B illustrates a cross-sectional surface profile of a substrate having semi-spherical troughs or grooves. FIG. 1C illustrates a cross-sectional surface profile of a substrate having pillars, or alternatively or in conjunction, wells. FIG. 1D illustrates a cross-sectional surface profile of a substrate having a coating. FIG. 1E illustrates a cross-sectional surface profile of a substrate having spherical particles. FIG. 1F illustrates a cross-sectional surface profile of FIG. 1B, with a first type of binders seeded or associated with the respective grooves. FIG. 1G illustrates a cross-sectional surface profile of FIG. 1B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9) rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary: the substrate may then be subjected to rotation (or other motion) following the dispensing of the solution. Alternatively, the substrate may be subjected to rotation (or other motion) prior to the dispensing of the solution: the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm. 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

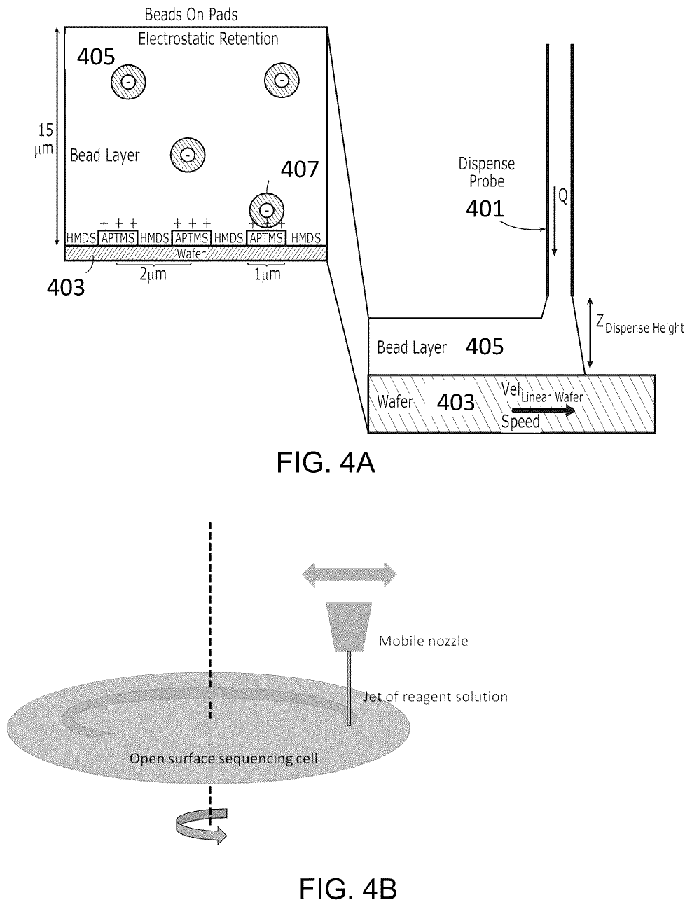

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 4B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm. 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

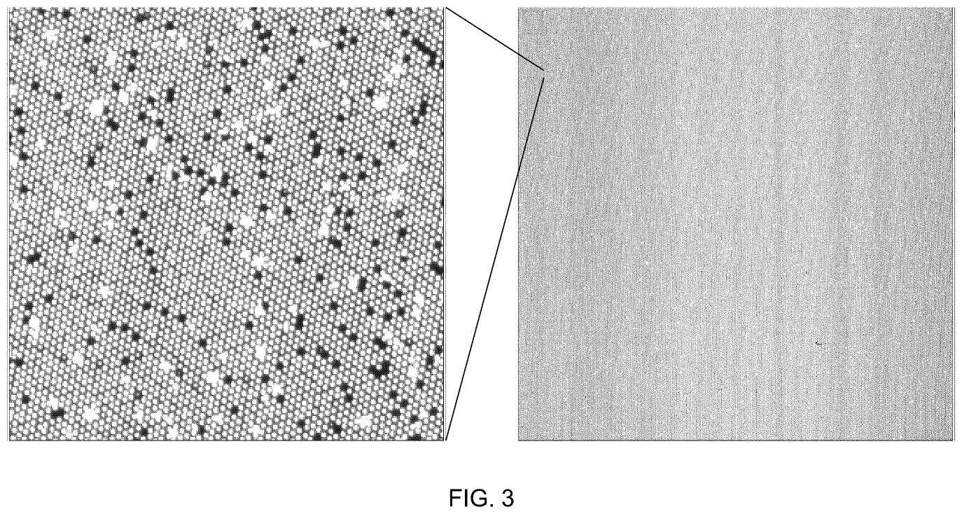

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 3 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 4A-4B. As shown in FIG. 4A, a solution comprising beads may be dispensed from a dispense probe 401 (e.g., a nozzle) to a substrate 403 (e.g., a wafer) to form a layer 405. The dispense probe may be positioned at a height (“Z”) above the substrate. In the illustrated example, the beads are retained in the layer 405 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 405 a bead may successfully land on a first location of the first location type (as in 407). In the illustrated example, the location size is 1 micron, the pitch between the different locations of the same location type (e.g., first location type) is 2 microns, and the layer has a depth of 15 micron. FIG. 4B illustrates a reagent (e.g., beads) being dispensed along a path on an open surface of the substrate. As shown in FIG. 4B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors. CCD cameras. CMOS cameras. TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

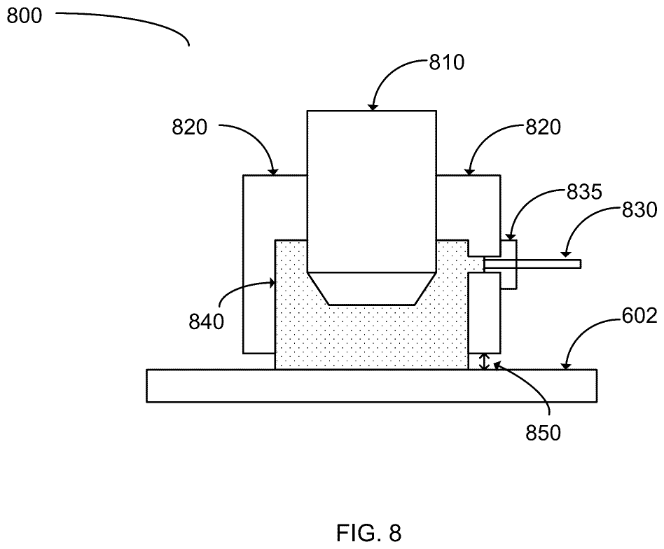

In some cases, the optical system may comprise an immersion objective lens. FIG. 8 shows a cross-sectional view of an immersion optical system 800. The system 800 may be used to optically image the substrates described herein. The system 800 may be integrated with any other optical system or system for nucleic acid sequencing described herein, or any element thereof. The system may comprise an optical imaging objective 810. The optical imaging objective may be an immersion optical imaging objective. The optical imaging objective may be configured to be in optical communication with a substrate, such as substrate 602 described herein. The optical imaging objective may be configured to be in optical communication with any other optical elements described herein. The optical imaging objective may be partially or completely surrounded by an enclosure 820. The enclosure may partially or completely surround a sample-facing end of the optical imaging objective. The enclosure and fluid may comprise an interface between the atmosphere in contact with the substrate and the ambient atmosphere. The atmosphere in contact with the substrate and the ambient atmosphere may differ in relative humidity, temperature, and/or pressure. The enclosure may have a generally cup-like shape or form. The enclosure may be any container. The enclosure may be configured to contain a fluid or immersion fluid 840 (such as water or an aqueous or organic solution) in which the optical imaging objective is to be immersed. The enclosure may be configured to maintain a minimal distance 850 between the substrate and the enclosure in order to avoid contact between the enclosure and the substrate during rotation of the substrate. In some instances, air or a pressure differential may be used to maintain the minimal distance. In some instances, the minimal distance is up to about 100 nm, 200 nm, 500 nm, 1 μm, 2 μm, 5 μm, 10 μm, 20 μm, 50 μm, 100 μm, 200 μm, 500 μm, 1 mm, or a distance that is within a range defined by any two of the preceding values. Even with the minimal distance, the enclosure 820 may contain the fluid due to surface tension effects. The system may comprise a fluid flow tube 830 configured to deliver fluid to the inside of the enclosure. The fluid flow tube may be connected to the enclosure through an adaptor 835. The adaptor may comprise a threaded adaptor, a compression adaptor, or any other adaptor. An electrical field application unit (not shown) can be configured to regulate a hydrophobicity of one or more surfaces of a container to retain at least a portion of the fluid contacting the immersion objective lens and the open substrate, such as by applying an electrical field.

As used herein, the fluid contacting the immersion objective lens may be referred to as “immersion fluid” or “fluid”. The immersion fluid may comprise any suitable immersion medium for imaging. For example, the immersion medium may comprise an aqueous solution. Non-limiting examples of aqueous immersion fluids include water. In some cases, the aqueous solution may comprise salts, surfactants, oils and/or any other chemicals or reagents useful in imaging. In some cases, the immersion medium comprises an organic solution. Non-limiting examples of organic immersion fluids include oils, perfluorinated polyethers, perfluorocarbons, and hydrofluorocarbons. In some cases, the immersion fluid may be substantially the same as the wash buffer, as described elsewhere herein, or any buffer used in the processes described herein. The immersion fluid may be tuned based on the optical requirements of the systems and methods described herein. For example, where a high numerical aperture (NA) is required, the appropriate immersion fluid (e.g., oil) may be used for imaging. In some cases, the immersion fluid may be selected to match an index of refraction of a solution on the substrate (e.g., a buffer), a surface (e.g., a coverslip or the substrate), or an optical component (e.g., an objective lens).

In some instances, the fluid may also be in contact with an open substrate. The optical imaging objective and enclosure may be configured to provide a physical barrier between a first location in which chemical processing operations are performed and a second location in which detection operations are performed. In this manner, chemical processing operations and detection operations may be performed with independent operation conditions. In addition, in such cases, contamination of the detector may be avoided.

The optical imaging objective may be in fluidic contact with the substrate (e.g., via the immersion fluid). The open substrate may comprise a layer of fluid covering the surface of the substrate. The optical imaging objective may be configured to scan the surface comprising the layer of fluid. The layer of fluid on the surface may comprise the same fluid as the immersion fluid. The layer of fluid on the surface may comprise a different fluid than the immersion fluid. The layer of fluid on the surface may be miscible with the immersion fluid, or the layer of fluid on the surface may be immiscible with the immersion fluid. In some cases, the layer of fluid is deeper where it contacts the optical imaging objective than at other points on the surface. A portion of the layer of fluid may adhere to the optical imaging objective. In some cases, the portion of the layer of fluid may move with the optical imaging objective relative to the substrate during scanning. The optical imaging objective may remain in fluidic contact with the substrate during scanning. The optical imaging objective may be configured to have a long travel distance in a vertical direction relative to the substrate. In some cases, the optical imaging objective may be configured to lift away from the substrate such that the optical imaging objective is no longer in fluidic contact with the substrate. For example, the optical imaging objective may be lifted away from the substrate while fluid is being dispensed on the substrate. A portion of the layer of fluid, the immersion fluid, or both may adhere to the optical imaging objective when it leaves fluidic contact with the substrate. The portion of the layer of fluid adhering to the optical imaging objective may prevent bubbles from forming or accumulating between the substrate and the optical imaging objective when the optical imaging objective re-enters fluidic contact with the substrate.

A system of the present disclosure may be contained in a container or other closed environment. For example, a container may isolate an internal environment from an external environment. The internal environment may be controlled such as to localize temperature, pressure, and/or humidity, as described elsewhere herein. In some instances, the external environment may be controlled. In some instances, the internal environment may be further partitioned, such as via, or with aid of, the enclosure 820 to separately control parts of the internal environment (e.g., first internal environment for chemical processing operations, second internal environment for detection operations, etc.). The different parts of the internal environment may be isolated via a seal. For example, the seal may comprise the immersion objective described herein.

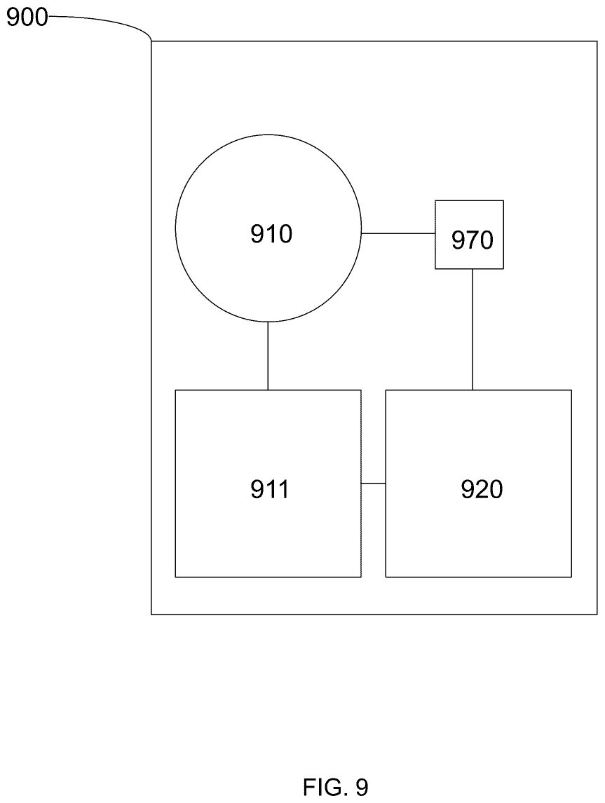

FIG. 9 shows a computerized system 900 for sequencing a nucleic acid molecule. The system may comprise a substrate 910, such as any substrate described herein. The system may further comprise a fluid flow unit 911. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 910. The fluid flow system may be configured to collect any solution from the substrate 910. The system may further comprise a detector 970, such as any detector described herein. The detector may be in sensing communication with the substrate surface.

The system may further comprise one or more processors 920. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in U.S. Patent Pub. No. 20210354126A1, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C. 30° C., 25° C., 20° C., or less. Different elements of the system may be maintained at different temperatures or within different temperature ranges, such as the temperatures or temperature ranges described herein. The systems (or any element thereof) may be maintained at a relative humidity of up to about 5%, 10%, 15%, 20%, 25%, 30%, 35%, 40%, 45%, 50%, 55%, 60%, 65%, 70%, 75%, 80%, 85%, 90%, 95%, or 100%. In some instances, the systems (or any element thereof) may be maintained at a relative humidity that is less than 5%. In some instances, the systems (or any element thereof) may be maintained at a relative humidity that is greater than 100%. In some instances, the systems (or any element thereof) may be maintained at a relative humidity that is within a range defined by any two of the preceding values.

Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

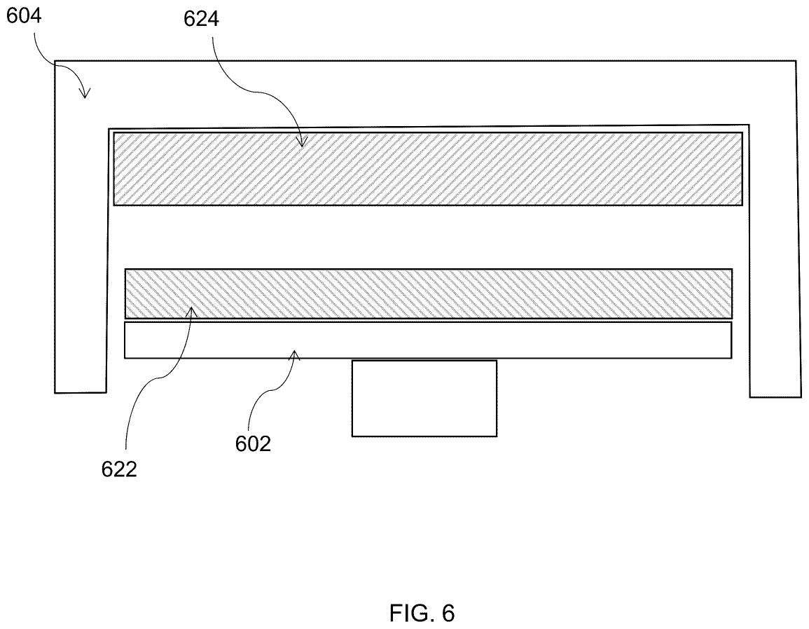

FIG. 6 illustrates a system with different environmental conditions in an open substrate system. An open substrate system may comprise a substrate 602 and a container 604 enclosing the substrate. The substrate 602 may be any substrate described herein. The container 604 may define a surrounding environment of the substrate 602. In some instances, the surrounding environment may be confined and/or closed. In some instances, the surrounding environment may be sealed (e.g., hermetically sealed, frictionally sealed, pneumatically, etc.). In some instances, the surrounding environment may be sealed using a pressure differential (e.g., pneumatic pressure, mechanical pressure, etc.). The open substrate system may comprise at least two non-overlapping regions, a first region 622 and a second region 624, having different environmental conditions. In some instances, the first region 622, contacting or in proximity to a surface of the substrate 602, such as the surface that comprises one or more analytes as described herein, may be maintained at a first set of temperatures and first set of humidities. In some instances, the second region 624, contacting or in proximity to a top portion of the container 604 (or otherwise referred to herein as a lid or cover), may be maintained at a second set of temperatures and second set of humidities. The first set of temperatures and first set of humidities may be controlled such as to prevent or minimize evaporation of one or more reagents on the surface of the substrate. In some instances, the systems or any element thereof (e.g., the first set of temperatures and first set of humidities) may be configured to prevent less than about 80%, 70%, 60%, 50%, 40%, 30%, 20%, 10%, 5%, 4%, 3%, 2%, or 1% evaporation of the volume of the solution layer dispensed on the uncovered surface. The second set of temperatures and second set of humidities may also be controlled such as to enhance or restrict condensation. For example, the first set of temperatures may be the lowest temperatures within the surrounding environment of the open substrate system. For example, the second set of temperatures may be the highest temperatures within the surrounding environment of the open substrate system. In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods. For example, the first region 622 may be maintained at cooler temperatures by controlling the temperature of the substrate 602, and the second region 624 may be maintained at warmer temperatures by controlling the temperature of a top portion of the container 604, via conduction.

The system may further comprise a reservoir beneath the substrate 602 (not shown in FIG. 6). The reservoir may be configured to hold fluid. The reservoir may be configured to collect fluid, precipitation, or condensation from other surfaces, for example from the substrate 602 or the top portion of the container 604. Fluid may be removed from the reservoir. In some cases, fluid may be removed from the reservoir volumetrically. For example, fluid may be removed from the reservoir volumetrically to balance an amount of fluid added to the system. In some cases, fluid is continuously added to the system and fluid is continuously removed from the reservoir. The amount of fluid added may be equal to the amount of fluid removed. In some cases, a volume of fluid in the reservoir is held constant. The volume of fluid in the reservoir may be determined based on a relative humidity of the system. The relative humidity of the system may depend on the volume of fluid in the reservoir, the amount of fluid in the system, the temperature of the system, or any combination thereof.

The system may be temperature controlled. In some cases, the elements of the system may be held at different temperatures. The differential temperatures of individual elements in the system may control the accumulation of condensation or precipitation on the individual elements of the system. The top portion of the container 604 may be held at a different temperature than the substrate 602, an objective of a detector, or the reservoir. Alternatively or in addition, the substrate may be held at a different temperature than the top portion of the container, the objective, or the reservoir. Alternatively or in addition, the reservoir may be held at a different temperature than the top portion of the container, the objective, or the substrate. Alternatively or in addition, the objective may be held at a different temperature than the top portion of the container, the reservoir, or the substrate. In some cases, the top portion of the container is held at a higher temperature than at least one other element in the system to prevent the accumulation of condensation on the top surface of the container. In an exemplary configuration, the top portion of the container is held at the highest temperature, the substrate is held at the lowest temperature, and the reservoir and the objective are held at intermediate temperatures, thereby preventing condensation from forming on the top portion of the container or from forming or dripping onto the objective. In another example, the objective is held at the highest temperature, the top portion of the container is held at an intermediate temperature, and the substrate and the reservoir are held at lower temperatures than the top portion of the container, thereby preventing condensation from forming on the top portion of the container or from forming or dripping onto the objective. In some cases, the objective may be fully or partially surrounded by a seal. The seal may be configured to prevent moisture from the container surrounding the substrate (for example, as shown in FIG. 6) from reaching other optical components in the system. The seal may comprise a flexible material. The flexible seal may be configured to allow relative motion of individual elements of the system while maintaining the seal. In some instances, the flexible seal may stretch, expand, or contract. For example, the flexible seal may be configured to allow independent motion of two or more imaging heads. Alternatively or in addition, the seal may comprise a waterproof material. For example, the seal may be rubber, silicone, latex, plastic, Teflon, nitrile, elastin, an elastomer, or a polymer. The seal may surround the objective and contact the top portion of the container. In some cases, a portion of the objective comprising a front lens is not covered by the seal. The front lens of the objective may be exposed to the container surrounding the substrate. In some cases, the front lens of the objective may be in fluidic contact with the substrate.

The systems (or any element thereof) may be contained within a sealed container, housing, or chamber that insulates the system (or any element thereof) from the external environment or atmosphere, allowing for the control of the temperature or humidity. An environmental unit (e.g., humidifiers, heaters, heat exchangers, compressors, etc.) may be configured to regulate one or more operating conditions in each environment. In some instances, each environment may be regulated by independent environmental units. In some instances, a single environmental unit may regulate a plurality of environments. In some instances, a plurality of environmental units may, individually or collectively, regulate the different environments. An environmental unit may use active methods or passive methods to regulate the operating conditions. For example, the temperature may be controlled using heating or cooling elements. The humidity may be controlled using humidifiers or dehumidifiers. In some instances, a part of the internal environment within the container or chamber may be further controlled from other parts of the internal environment. Different parts may have different local temperatures, pressures, and/or humidity. For example, the internal environment may comprise a first internal environment and a second internal environment separated by a seal. Alternatively or in conjunction, the systems or methods described herein may comprise a solution comprising an agent that may reduce evaporation. For example, the solution may comprise glycerol, which can prevent evaporation of the solution.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below: adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

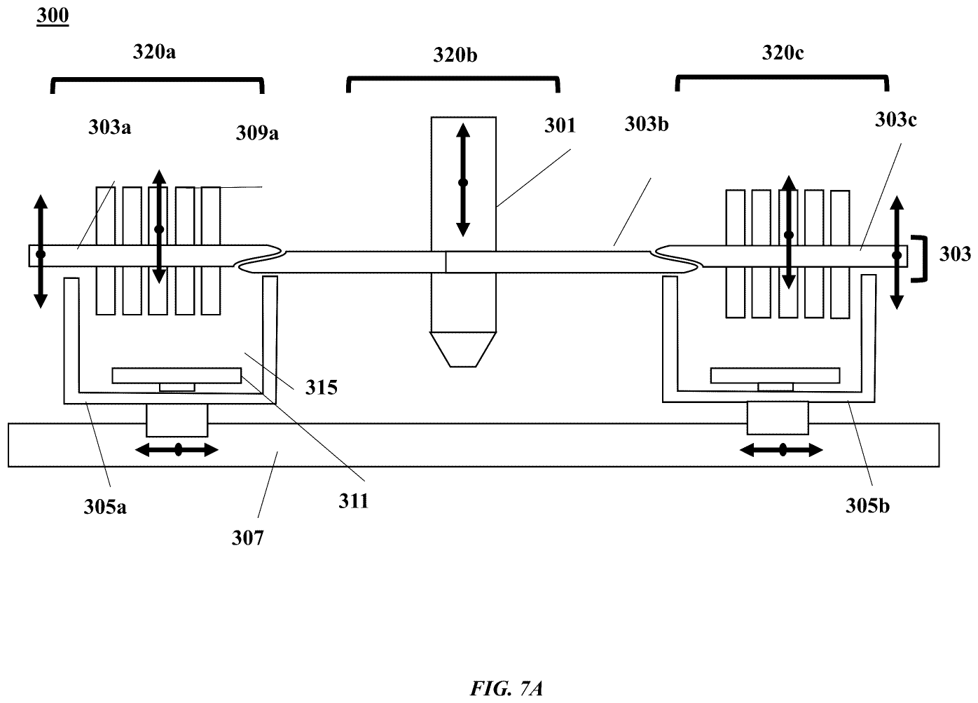

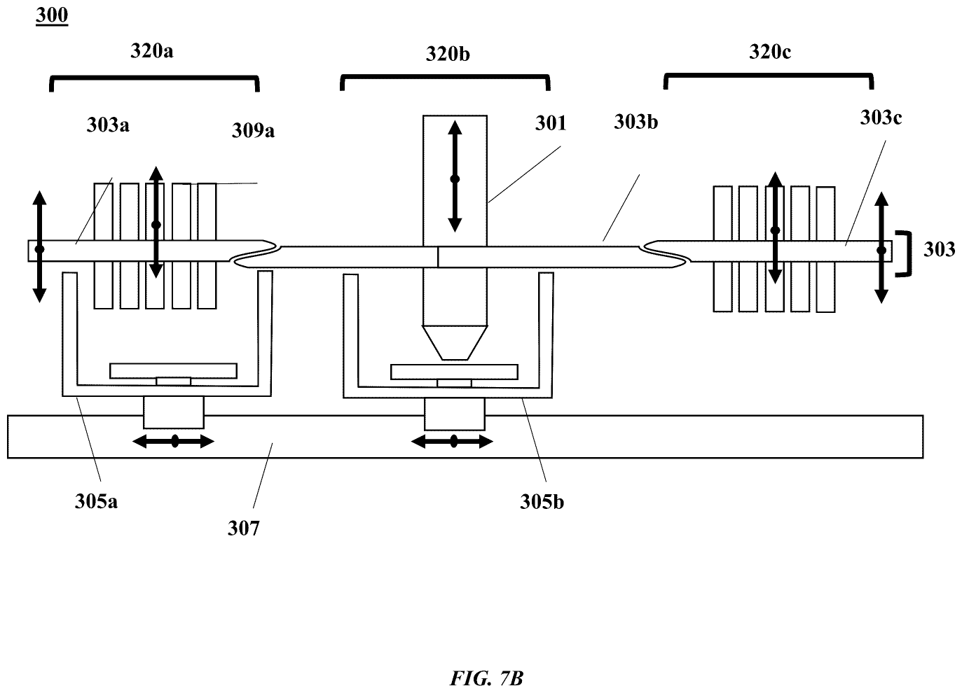

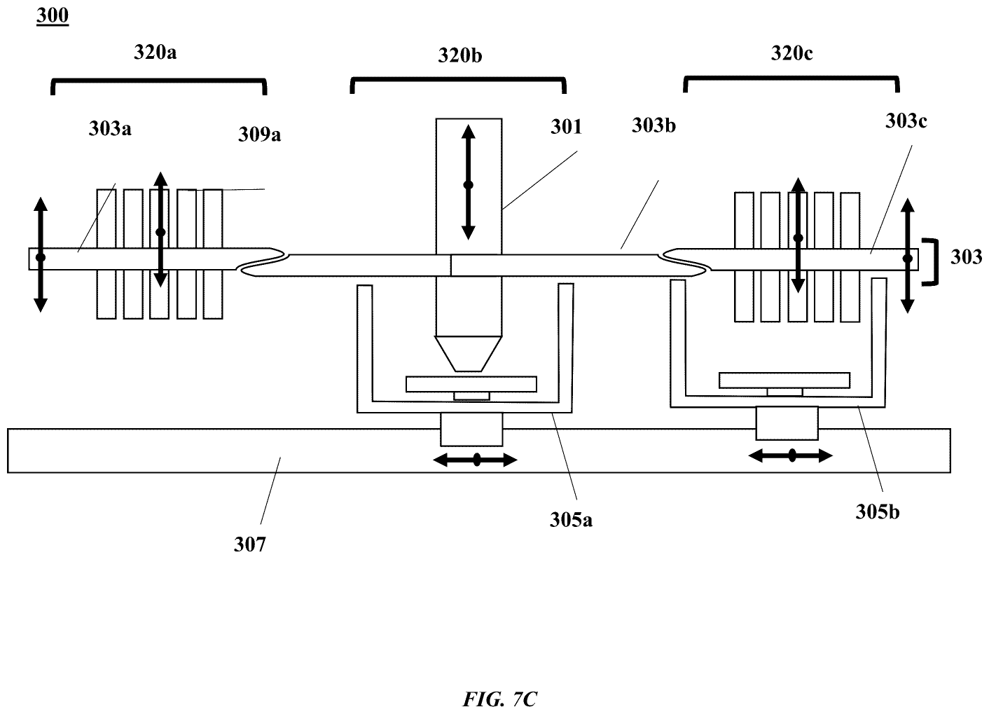

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Nucleic Acid Sequencing

Without intending to be bound by theory, the methods, systems, and compositions provided herein may facilitate improved sequencing of nucleic acid molecules.

In some instances, a method for sequencing may employ sequencing by synthesis schemes wherein a nucleic acid molecule is sequenced base-by-base with primer extension reactions. For example, a method for sequencing a nucleic acid molecule may comprise providing a substrate comprising an array having immobilized thereto the nucleic acid molecule. The method may comprise directing a solution comprising a plurality of nucleotides across the array. The nucleic acid molecule may be subjected to a primer extension reaction under conditions sufficient to incorporate or specifically bind at least one nucleotide from the plurality of nucleotides into a growing strand that is complementary to the nucleic acid molecule (e.g., extending primer molecule). A signal indicative of incorporation or binding of at least one nucleotide may be detected, thereby sequencing the nucleic acid molecule. The process of extension of the growing strand and detection may be repeated for any number of times to obtain a desired length of sequence information.

Optionally, a substrate may be rotated according to the systems and methods described elsewhere herein to facilitate the efficiency of chemistry (e.g., extension of primer) and/or detection operations.

In some instances, the method may comprise, prior to providing the substrate having immobilized thereto the nucleic acid molecule, immobilizing the nucleic acid molecule to the substrate. In an example, a solution comprising a plurality of nucleic acid molecules comprising the nucleic acid molecule may be directed to the substrate prior to, during, or subsequent to rotation of the substrate, and the substrate may be subject to conditions sufficient to immobilize at least a subset of the plurality of nucleic acid molecules as an array on the substrate. Rotation of the substrate may facilitate coating of the substrate surface with the solution.

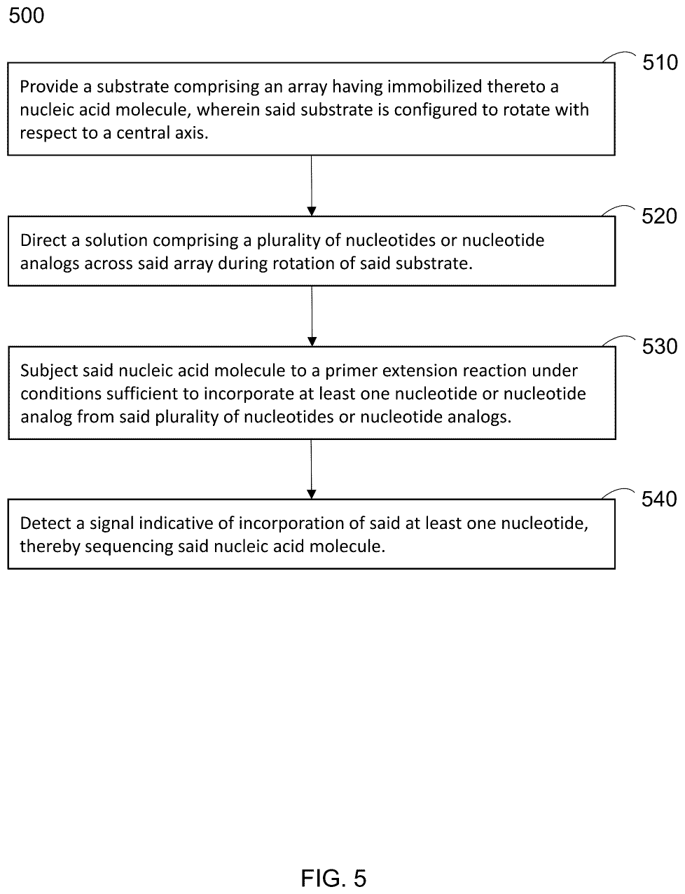

FIG. 5 illustrates an example method 500 for sequencing a nucleic acid molecule. In some instances, the method may comprise providing 510 a substrate. The substrate may be configured to rotate with respect to an axis. The axis may be an axis through the center or substantially center of the substrate. The axis may be an off-center axis. The substrate may be configured to rotate with different rotational velocities during different operations described herein. The substrate may be configured to rotate with a rotational velocity that varies according to a time-dependent function, such as a ramp, sinusoid, pulse (e.g., thereby creating an annular wave of the solution moving radially outward), or other function or combination of functions. The time-varying function may be periodic or aperiodic.

In some instances, the method further comprises directing a solution across the substrate. In some instances, the solution is directed across the substrate prior to or during rotation of the substrate. The solution may be centrifugally directed across the substrate. In some instances, the solution may be directed to the solution during rotation of the substrate in a continuous stream while the stream moves radially with respect to an axis of rotation of the substrate, thereby directing the solution to the array in a spiral pattern.

In some cases, a solution may comprise beads, as described elsewhere herein. The beads may be coated with a nucleic acid molecule to be sequenced. The solution comprising beads may be dispensed onto the substrate using the methods described herein. For example, the solution comprising beads may be dispensed onto the substrate, as illustrated in FIGS. 4A-4B. In some cases, the beads may preferentially interact with a first location type of the substrate (e.g., a positively charged region), as illustrated in FIG. 4A. In some cases, a bead may not interact with a second location type of the substrate (e.g., a hydrophobic region). For example, a bead coated with a nucleic acid molecule may interact with the first location type, and a bead that is not coated with a nucleic acid molecule may not interact with the first location type, as shown in FIG. 4B. A solution comprising beads may be dispensed onto the substrate to initially load the substrate with the template nucleic acid molecules to be sequenced.

In some cases, the solution may comprise probes configured to interact with nucleic acid molecules (e.g., initially loaded, e.g., with the beads). For example, in some instances, such as for performing sequencing by synthesis, the solution may comprise a plurality of nucleotides (in single bases). The plurality of nucleotides may include nucleotide analogs, naturally occurring nucleotides, and/or non-naturally occurring nucleotides, collectively referred to herein as “nucleotides.” The plurality of nucleotides may or may not be bases of the same canonical base type (e.g., A, T, G, C, etc.). For example, the solution may or may not comprise bases of only one type. The solution may comprise at least 1 or more type of bases. For example, the solution may comprise at least 2, 3, 4, 5, 6, 7, 8, 9, or 10 types of nucleotide bases. For instance, the solution may comprise any possible mixture of A, T, C, and G, or subset thereof. In some instances, the solution may comprise a plurality of natural nucleotides and non-natural nucleotides. The plurality of natural nucleotides and non-natural nucleotides may or may not be bases of the same type (e.g., A, T, G, C). In some cases, the solution may comprise probes that are oligomeric (e.g., oligonucleotide primers). For example, in some instances, such as for performing sequencing by synthesis, the solution may comprise a plurality of nucleic acid molecules, e.g., primers, that comprise 1, 2, 3, 4, 5, 6, 7, 8, 9, 10, 20, 30, 40, 50, 60, 70, 80, 90, 100 or more nucleotide bases. The plurality of nucleic acid molecules may comprise nucleotide analogs, naturally occurring nucleotides, and/or non-naturally occurring nucleotides, collectively referred to herein as “nucleotides.”

In some cases, the plurality of nucleotides in the solution may be non-terminated. In some cases, none of the nucleotides in the solution may be terminated. Following incorporation of a non-terminated nucleotide into a nucleic acid strand, the nucleic acid strand may be able to incorporate another nucleotide. For example, where a solution of non-terminated A-base nucleotides are provided to a template that comprises a poly-T sequence, at the poly-T sequence locations the nucleic acid strand may incorporate multiple non-terminated A-base nucleotides in consecution. Alternatively, one or more nucleotides of the plurality of nucleotides may be terminated (e.g., reversibly terminated). For example, a nucleotide may comprise a reversible terminator, or a moiety that is capable of terminating primer extension reversibly. Nucleotides comprising reversible terminators may be accepted by polymerases and incorporated into growing nucleic acid sequences analogously to non-reversibly terminated nucleotides. A polymerase may be any naturally occurring (i.e., native or wild-type) or engineered variant of a polymerase (e.g., DNA polymerase, Taq polymerase, etc.). Following incorporation of a nucleotide analog comprising a reversible terminator into a nucleic acid strand, the reversible terminator may be removed to permit further extension of the nucleic acid strand.

In some cases, the solution may comprise any reagent described herein, such as a reagent configured to improve photometry for detection, a reagent configured to reduce particle aggregation, and/or a reagent configured to shrink or otherwise decrease the size of particles. A solution may comprise any number of types of components and/or reagents (e.g., beads, nucleotides, nucleic acid molecules, primers, buffer, particle shrinking reagent, etc.).

The solution may be directed to the array using one or more nozzles. In some cases, different reagents (e.g., nucleotide solutions of different types, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Two or more nozzles may be operated in combination to deliver fluids to the substrate more efficiently.

In some instances, the solution may be dispensed on the substrate while the substrate is stationary. In some instances, the substrate may then be subjected to rotation following the dispensing of the solution. Alternatively, in some cases, the substrate may be subjected to rotation prior to the dispensing of the solution: the solution may then be dispensed on the substrate while the substrate is rotating.

In some instances, the method further comprises subjecting 530 a nucleic acid molecule on the substrate to a primer extension reaction. The primer extension reaction may be conducted under conditions sufficient to incorporate at least one nucleotide from the plurality of nucleotides into a growing strand that is complementary to the nucleic acid molecule. The incorporated nucleotide may or may not be labeled. In some cases, operation 530 may further comprise modifying at least one nucleotide. Modifying the nucleotide may comprise labeling the nucleotide. For instance, the nucleotide may be labeled, such as with a dye, fluorophore, or quantum dot. The nucleotide may be cleavably labeled (e.g., labeled with a removable label). In some instances, modifying the nucleotide may comprise activating (e.g., stimulating) a label of the nucleotide.

In some instances, the method further comprises detecting 540 a signal indicative of incorporation of the at least one nucleotide. The signal may be an optical signal. The signal may be a fluorescence signal. The signal may be detected during rotation of the substrate. The signal may be detected following termination of the rotation. The signal may be detected while the nucleic acid molecule to be sequenced is in fluid contact with the solution. The signal may be detected following fluid contact of the nucleic acid molecule with the solution. Operation 540 may further comprise modifying a label of the at least one nucleotide. For instance, operation 540 may further comprise cleaving the label of the at least one nucleotide (e.g., after detection). The at least one nucleotide may be cleaved by one or more stimuli, such as exposure to a chemical, an enzyme, light (e.g., ultraviolet light), or heat. In some cases, once the label is cleaved, a signal indicative of the incorporated nucleotide may not be detectable with one or more detectors.

In some instances, method 500 may further comprise repeating operations 520, 530, and/or 540 one or more times to identify one or more additional signals indicative of incorporation of one or more additional nucleotides, thereby sequencing the nucleic acid molecule. Operations 520, 530, and 540 may be collectively referred to herein as a “flow cycle.” In some instances, method 500 may comprise repeating operations 520, 530, and/or 540 in an iterative manner (e.g., iterative flow cycles). For each iteration (e.g., flow cycle), an additional signal may indicate incorporation of an additional nucleotide. The additional nucleotide may be the same nucleotide as detected in the previous iteration. The additional nucleotide may be a different nucleotide from the nucleotide detected in the previous iteration. In some instances, at least one nucleotide may be modified (e.g., labeled and/or cleaved) between each iteration of the operations 520, 530, or 540. For instance, method 500 may comprise repeating the operations 520, 530, and/or 540 at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, 1,000,000,000 or more times. The method 500 may comprise repeating the operations 520, 530, and/or 540 a number of times that is within a range defined by any two of the preceding values. The method 500 may thus result in the sequencing of a nucleic acid molecule of any size.

The method may comprise directing different solutions to the array during rotation of the substrate in a cyclical manner. For instance, the method may comprise directing a first solution containing a first type of nucleotide (e.g., in a plurality of nucleotides of the first type) to the array, followed by a second solution containing a second type of nucleotide, followed by a third type of nucleotide, followed by a fourth type of nucleotide, etc. In another example, different solutions may comprise different combinations of types of nucleotides. For example, a first solution may comprise a first canonical type of nucleotide (e.g., A), a second solution may comprise a second canonical type of nucleotide (e.g., C), a third solution may comprise a third canonical type of nucleotide (e.g., T), and a fourth solution may comprise a fourth canonical type (e.g., G) of nucleotide. In another example, a first solution may comprise a first canonical type of nucleotide (e.g., A) and a second canonical type of nucleotide (e.g., C), and a second solution may comprise the first canonical type of nucleotide (e.g., A) and a third canonical type of nucleotide (e.g., T), and a third solution may comprise the first canonical type, second canonical type, third canonical type, and a fourth canonical type (e.g., G) of nucleotide. In another example, a first solution may comprise a mixture of labeled and unlabeled nucleotides, and a second solution may comprise unlabeled nucleotides. In another example, a first solution may comprise labeled nucleotides, and a second solution may comprise unlabeled nucleotides, and a third solution may comprise a mixture of labeled and unlabeled nucleotides. In another example, a first solution may comprise a mixture of labeled and unlabeled nucleotides of a first canonical base type (e.g., A), a second solution may comprise unlabeled nucleotides of the first canonical base type (e.g., A), a third solution may comprise a mixture of labeled and unlabeled nucleotides of a second canonical base type (e.g., C), a fourth solution may comprise unlabeled nucleotides of the second canonical base type (e.g., C), a fifth solution may comprise a mixture of labeled and unlabeled nucleotides of a third canonical base type (e.g., T), a sixth solution may comprise unlabeled nucleotides of the third canonical base type (e.g., T), a seventh solution may comprise a mixture of labeled and unlabeled nucleotides of a fourth canonical base type (e.g., G), and an eighth solution may comprise unlabeled nucleotides of the fourth canonical base type (e.g., G). It will be appreciated that the sequence and/or cycle of nucleotide base types that are flowed to the substrate may be flexibly customized and varied for particular sequencing applications.

In some instances, the method may comprise directing at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, 10,000,000, 20,000,000, 50,000,000, 100,000,000, 200,000,000, 500,000,000, or 1,000,000,000 types of solutions to the array. The method may comprise directing a number of types of solutions that is within a range defined by any two of the preceding values to the array.

The method may comprise directing at least 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, at least 10,000,000, 20,000,000, 50,000,000, 100,000,000, 200,000,000, 500,000,000, or 1,000,000,000 washing solutions to the substrate. For instance, a washing solution may be directed to the substrate after each type of nucleotide is directed to the substrate. The washing solutions may be distinct. The washing solutions may be identical. The washing solution may be dispensed in pulses during rotation, creating annular waves as described herein. The washing solution may be dispensed in a continuous stream during rotation while the stream moves radially with respect to the axis of rotation of the substrate, thereby dispensing the washing solution in a spiral pattern.

The method may further comprise recycling a subset or an entirety of the solution(s) after the solution(s) have contacted the substrate. Recycling may comprise collecting, filtering, and reusing the subset or entirety of the solution. The filtering may be molecule filtering.

In some cases, the operations 520 and 530 may occur at a first location (e.g., first station) and the operation 540 may occur at a second location (e.g., second station). The first and second locations may comprise first and second processing stations, respectively, as described herein. The first and second locations may comprise or use first and second rotating spindles. The first rotating spindle may be exterior or interior to the second rotating spindle. The first and second rotating spindles may be configured to rotate with different angular velocities. Alternatively, the operation 520 may occur at a first location and the operations 530 and 540 may occur at the second location.

The method may further comprise transferring the substrate between the first and second locations. In some instances, operations 520 and 530 may occur while the substrate is rotated at a first angular velocity and operation 540 may occur while the substrate is rotated at a second angular velocity. In some cases, the first angular velocity may be less than the second angular velocity, in some cases, the first angular velocity may be between about 0) rpm and about 100 rpm. The second angular velocity may be between about 100 rpm and about 1,000 rpm. Alternatively, in some instances, the operation 520 may occur while the substrate is rotated at the first angular velocity and the operations 530 and 540 may occur while the substrate is rotated at the second angular velocity.

Many variations, alterations, and adaptations based on the method 500 provided herein are possible. For example, the order of the operations of the method 500 may be changed, some of the operations removed, some of the operations duplicated, and additional operations added as appropriate. Some of the operations may be performed in succession. Some of the operations may be performed in parallel. Some of the operations may be performed once. Some of the operations may be performed more than once. Some of the operations may comprise sub-operations. Some of the operations may be automated. Some of the operations may be manual. Some of the operations may be performed separately, e.g., in different locations or during different steps and/or processes. For example, directing a solution comprising a plurality of probes to the substrate may occur separately from the reaction and detection processes. Various sequencing approaches, including non-sequencing-by-synthesis approaches, may be implemented.

For example, in some cases, in the third operation 530, instead of facilitating a primer extension reaction, the nucleic acid molecule may be subject to conditions to allow transient binding of a nucleotide from the plurality of nucleotides to the nucleic acid molecule. The transiently bound nucleotide may be labeled. The transiently bound nucleotide may be removed, such as after detection (e.g., see operation 540). Then, a second solution may be directed to the substrate, this time under conditions to facilitate the primer extension reaction, such that a nucleotide of the second solution is incorporated (e.g., into a growing strand hybridized to the nucleic acid molecule). The incorporated nucleotide may be unlabeled. After washing, and without detecting, another solution of labeled nucleotides may be directed to the substrate, such as for another cycle of transient binding.

In another example, such as for performing sequencing by ligation, the solution may comprise different probes. For example, the solution may comprise a plurality of oligonucleotide molecules. For example, the oligonucleotide molecules may have a length of about 2, 3, 4, 5, 6, 7, 8, 9, 10 or more bases. The oligonucleotide molecules may be labeled with a dye (e.g., fluorescent dye), as described elsewhere herein. In some instances, such as for detecting repeated sequences in nucleic acid molecules, such as homopolymer repeated sequences, dinucleotide repeated sequences, and trinucleotide repeated sequences, the solution may comprise targeted probes (e.g., homopolymer probe) configured to bind to the repeated sequences. The solution may comprise one type of probe (e.g., nucleotides). The solution may comprise different types of probes (e.g., nucleotides, oligonucleotide molecules, etc.). The solution may comprise different types of probes (e.g., oligonucleotide molecules, antibodies, etc.) for interacting with different types of analytes (e.g., nucleic acid molecules, proteins, etc.). Different solutions comprising different types of probes may be directed to the substrate any number of times, with or without detection between consecutive cycles (e.g., detection may be performed between some consecutive cycles, but not between some others), to sequence or otherwise process the nucleic acid molecule, depending on the type of processing.

As such, a template nucleic acid molecule may be processed using a substrate comprising an array having immobilized thereto the template nucleic acid molecule. The template nucleic acid molecule may be a sample nucleic acid molecule derived from a nucleic acid sample (e.g., as described herein). The template nucleic acid molecule may be immobilized to the substrate via a particle (e.g., bead), such as a particle treated according to any of the methods provided herein. The template nucleic acid molecule may be hybridized to a growing nucleic acid strand (e.g., an extending primer molecule). The substrate may comprise a plurality of template nucleic acid molecules (e.g., across a plurality of particles) immobilized thereto, such as in an array. The substrate may be configured to rotate with respect to a rotational axis. A solution comprising a plurality of nucleotides or nucleotide analogs may be directed across the array during rotation of the substrate. The plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with an optically detectable label such as a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. The template nucleic acid molecule and growing nucleic acid strand complex may be subjected to conditions sufficient for nucleotides or nucleotide analogs of the plurality of nucleotides or nucleotide analogs to be incorporated into the growing nucleic acid strand (e.g., in a primer extension reaction). A signal (e.g., an optical signal) indicative of incorporation of a nucleotide or nucleotide analog may be detected (e.g., via optical detection), thereby sequencing the nucleic acid molecule. The plurality of nucleotides or nucleotide analogs may be provided to the growing nucleic acid strands in a first reaction mixture, followed by one or more additional flows to wash away unbound nucleotides or nucleotide analogs and reagents from the reaction space and/or to cleave cleavable moieties of linkers coupling labels to nucleotides or nucleotide analogs, etc. Additional reaction mixtures comprising different combinations of nucleotides or nucleotide analogs may be provided (e.g., in a predefined sequence) to continue sequencing of the template nucleic acid molecule.

In another example, a template nucleic acid molecule may be processed using an open substrate comprising an array of immobilized analytes thereon. For example, a template nucleic acid molecule may be immobilized to the open substrate via a particle (e.g., bead), such as a particle treated according to any of the methods provided herein. The template nucleic acid molecule may be hybridized to a growing nucleic acid strand (e.g., an extending primer molecule). The open substrate may be configured to rotate with respect to a rotational axis. A solution comprising a plurality of probes (e.g., nucleotides or nucleotide analogs) may be delivered to a region proximal to the central axis and/or rotational axis of the open substrate to introduce the solution to the open substrate. The solution may be dispersed across the open substrate such that at least one of the plurality of probes binds to at least one of the immobilized analytes to form a bound probe. Where the plurality of probes is a plurality of nucleotides or nucleotide analogs, the plurality of nucleotides or nucleotide analogs may comprise non-terminated nucleotides to facilitate sequencing of homopolymeric regions of a template nucleic acid molecule. The plurality of nucleotides or nucleotide analogs may comprise a plurality of labeled nucleotides or nucleotide analogs labeled with a fluorescent label (e.g., coupled to a nucleotide or nucleotide analog via a linker, such as a semi-rigid linker comprising a cleavable moiety). The plurality of nucleotides or nucleotide analogs may comprise nucleotides or nucleotide analogs of a single canonical type (e.g., adenine, uracil, thymine, cytosine, or guanine-containing nucleotides or nucleotide analogs) or of one or more different types. A bound probe may comprise a growing nucleic acid strand having a nucleotide or nucleotide analog incorporated therein. Formation of the bound probe may comprise subjecting a template nucleic acid molecule to conditions sufficient for nucleotides or nucleotide analogs of the plurality of nucleotides or nucleotide analogs to be incorporated into the growing nucleic acid strand (e.g., in a primer extension reaction).

### Particle Shrinkage, Aggregation, and Loading Efficiency

Described herein are methods, systems, and compositions to improve processing of particles on a substrate. In some cases, particles may be treated prior to, during, or subsequent to loading onto a substrate. Alternatively or in addition, a substrate may be treated prior to, during, or subsequent to loading particles onto the substrate. Treatment may comprise contacting the particles and/or the substrate with a buffer solution. Treatment may comprise incubation of the particles in or with the buffer solution. Treatment of the particles, on or off the substrate, may effectively decrease an average size or dimension of the particles. The decreased size of the particles may be advantageous to one or more sequencing operations.

In one example, a plurality of particles may be loaded onto individually addressable locations of a substrate, the nucleic acid molecules attached to the particles subjected to a chemistry operation (e.g., primer extension reaction) to label the particles, and the substrate or at least a portion thereof imaged; treating the particles with a buffer solution prior to imaging may decrease the average size or dimension of the particles, improving photometry quality. In some cases, the particles may be treated with the buffer solution at each imaging step in the sequencing cycles, or only at a subset of imaging steps in the sequencing cycles. In some cases, the particles may be treated with the buffer solution at only one or more specific imaging steps, such as the first imaging step, first two imaging steps, first three imaging steps, first four imaging steps, or any number of preamble imaging steps, sufficient for one or more processors to process the signals to complete an initial detection of the particle locations on the substrate and build a particle catalogue: the particle catalogue can be referenced to attribute signals detected at subsequent imaging steps (for subsequent flows) to specific particles or locations thereof.

In another example, a plurality of particles may be loaded onto individually addressable locations of a substrate, the nucleic acid molecules attached to the particles subjected to a chemistry operation (e.g., primer extension reaction) to label the particles, and the substrate or at least a portion thereof imaged; treating the particles with a buffer solution prior to, during, or subsequent to loading the particles onto the substrate may decrease the average size or dimension of the particles, allowing the substrate to immobilize more particles onto individually addressable locations of the substrate (e.g., at higher density) with reduced particle aggregation. In some cases, the particles may be loaded on the substrate in two or more loading steps, with intervening buffer solution treatment of particles. In some cases, the particles may be incubated in a buffer solution for a predetermined duration prior to loading to pre-shrink the particles.

### Improving Photometry

A method for processing a substrate may comprise providing the substrate comprising a plurality of individually addressable locations and adding a buffer solution to the substrate. In some cases, a method for processing a substrate may comprise providing the substrate comprising a plurality of individually addressable locations, and adding a buffer solution, across an air gap, to the substrate. A plurality of particles may be immobilized to at least a subset of a plurality of individually addressable locations. The buffer solution may decrease an average size of at least a subset of the plurality of particles. In some instances, the buffer solution comprises polyethylene glycol (PEG). In some instances, the buffer solution comprises spermine. In some instances, the buffer solution comprises MgCl₂. In some instances, the buffer solution comprises both PEG and MgCl₂.

The plurality of particles may be immobilized on the substrate prior to adding the buffer solution on the substrate. The plurality of particles may be immobilized on the substrate sequent to adding the buffer solution on the substrate, that is, such that the particles are loaded onto a substrate that comprises the buffer solution. In some cases, the plurality of particles may be mixed with the buffer solution, such as incubated in the buffer solution, prior to loading the plurality of particles in the buffer solution to the substrate.

In some cases, the substrate may be substantially planar. In some cases, the plurality of individually addressable locations may comprise protrusions from a base surface of the substrate. In some cases, a plurality of particles may be electrostatically immobilized to at least a subset of the plurality of individually addressable locations.

The method for processing a substrate may further comprise imaging at least a portion of the substrate prior to, during, and/or subsequent to adding a buffer solution to the substrate. The buffer solution may be present on the substrate prior to, during, and/or subsequent to imaging. In some cases, imaging may detect at least about 100,000 particles/cm² of the substrate. In some cases, imaging may detect up to about 10,000, 50,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,100,000, 1,200,000, 1,300,000, 1,400,000, 1,500,000, 1,600,000, 1,700,000, 1,800,000, 1,900,000, 2,000,000 particles/cm² of the substrate or more. In some cases, imaging may detect a number of particles/cm² of the substrate within a range defined by any two of the preceding values.

The imaging may detect a plurality of particles on the substrate. In some instances, imaging may detect up to about 10, 100, 1000, 10,000, 100,000, 1,000,000, 10,000,000, 100,000,000, 1,000,000,000, 10,000,000,000, 100,000,000,000 or more particles. In some cases, imaging may detect more than about 100,000,000,000 particles. In some cases, imaging may detect less than 10 particles. In some instances, imaging may detect at least a subset of a plurality of particles on the substrate. In some cases, imaging may detect up to about 5%, 10%, 15%, 20%, 25%, 30%, 35%, 40%, 45%, 50%, 55%, 60%, 65%, 70%, 75%, 80%, 85%, 90%, 95%, 99%, or more of a plurality of particles. In some cases, imaging may detect less than 5% of a plurality of particles. In some cases, imaging may detect more than 99% of a plurality of particles.

In some instances, the buffer solution may comprise a polymer. In some cases, the buffer solution may comprise polyethylene glycol (PEG). In some cases, a PEG molecule may comprise one or more subunits. In some cases, a PEG molecule may comprise a polymer of one or more subunits. In some cases, a PEG molecule may comprise a homopolymer of one or more subunits. In some cases, a PEG molecule may comprise a heteropolymer of one or more subunits.

In some cases, a subunit of PEG may comprise ethylene glycol, ethylene oxide, a derivative thereof, or a combination thereof. In some cases, a PEG molecule may comprise polyethylene oxide (PEO), polyoxyethylene (POE), a derivative thereof, or a combination thereof.

In some cases, a PEG molecule may be represented by Formula I:

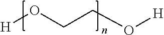

wherein n is the number of units.

In some cases, a molecular weight (M.W.) of a PEG molecule may be calculated by Formula II: M.W. of PEG (gram/molecule)=44.05n+18.02, where n is the same as the number of units as in Formula I.

In some instances, the molecular weight of a PEG molecule may be used to calculate the number of units. In other cases, the number of units (e.g., n of Formula I or II) may be used to calculate the molecular weight of a PEG molecule.

In some instances, a PEG molecule may be represented by the chemical formula H—(O—CH2-CH2)ₙ—OH or C2ₙH4ₙ+2Oₙ+1, where n is the number of unit. In some cases, a PEG molecule may also be represented by chemical formula H—(O—CH2-CH2)ₙ—OH or C2ₙH4ₙ+2Oₙ+1, where n is the number of units.

In some cases, a PEG molecule may be represented as PEG-n. In some instances, n comprises the molecular mass of the PEG molecule. For example, PEG-4000 may be a PEG molecule with a molecular mass of about 4000 daltons (Da), while PEG-8000 may be a PEG molecule with a molecular mass of about 8000 Da. In some cases, a population of PEG molecules may also be represented as PEG-n, where n may comprise the average molecular mass of the population of PEG molecules. For example, PEG-4000 may be a population of PEG molecules with an average molecular mass of about 4000 Da, while PEG-8000 may be a population of PEG molecules with an average molecular mass of about 8000 Da.

In some instances, a PEG molecule may have a molecular mass of up to about 100, 200, 300, 400, 500, 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, or more Da. In some cases, a PEG molecule may have a molecular mass of more than about 20,000 Da. In some cases, a PEG molecule may have a molecular mass of less than about 100 Da. In some instances, a PEG molecule may have a molecular mass within a range defined by any two of the preceding values. In some cases, a PEG molecule may have a molecular weight of at least about 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, 1×10⁸ or more grams per molecule (g/mol). In some cases, a PEG molecule may have a molecular weight of more than about 1×10⁸ g/mol. In some cases, a PEG molecule may have a molecular weight of less than about 1×10⁴ g/mol. In some cases, a PEG molecule may have a molecular weight within a range defined by any two of the preceding values.

In some cases, n comprises the number of units of PEG, such as n number of units as indicated in Formula I. For example, PEG-4000 may be a PEG molecule with about 4000 subunits, while PEG-8000 may be a PEG molecule with about 8000 subunits. In some cases, a population of PEG molecules may also be represented as PEG-N, where N may comprise the average number of units of the population of PEG molecules, such as n in Formula I. For example, PEG-4000 may be a population of PEG molecules with an average of about 4000 subunits, while PEG-8000 may be a population of molecules of about 8000 subunits.

In some instances, the PEG concentration may be up to about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, by weight, of a buffer solution. In some cases, the PEG concentration may be less than about 0.1% by weight, of a buffer solution. In some cases, the PEG concentration may be more than about 50% by weight, of a buffer solution. In some instances, the PEG concentration may be a percent by weight of a buffer solution within a range defined by any two of the preceding values. In some cases, the PEG concentration may be up to about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, by volume, of a buffer solution. In some cases, the PEG concentration may be less than about 0.1%, by volume, of a buffer solution. In some cases, the PEG concentration may be more than about 50%, by volume, of a buffer solution. In some instances, the PEG concentration may be a percent by volume of a buffer solution within a range defined by any two of the preceding values.

In some cases, a PEG molecule with different number of units (or a PEG of a different molar mass) may exhibit different properties. In some cases, PEG molecules may be liquids or solids (e.g., crystalized forms of PEG molecules). In some instances, a PEG molecule may comprise a glass transition temperature (T_{g}) from about −40° C., to about −70° C. In some cases, a PEG molecule may be dissolved in polar or nonpolar solvents. In some instances, a PEG molecule may be dissolved in hydrophilic solvents. In some instances, a PEG molecule may dissolve in organic solvents, such as alcohol, methylene chloride, acetone, toluene, acetonitrile, benzene, dichloromethane, chloroform, or a combination herein and thereof. In other cases, a PEG molecule may be amphiphilic. In some instances, a PEG molecule may have a branched, star, linear, comb-like, structure; or any combinations thereof. In some instances, a PEG molecule may comprise a terminal hydroxyl group. In other cases, a terminal hydroxyl group of a PEG molecule may be converted into a symmetric or asymmetric functional group.

In some instances, the buffer solution may comprise spermine. In some cases, the buffer solution may comprise one or more nitrogen atoms. In some cases, the buffer solution may comprise one or more amine groups. In some cases, the buffer solution may comprise a tetramine. In some cases, the buffer solution may comprise an alkane. In some cases, the buffer solution may comprise a polyalkane. In some cases, the buffer solution may comprise a tetradecane. In some cases, the buffer solution may comprise a polyazaalkane. In some cases, spermine may have nitrogen atoms replacing carbon atoms one or more of positions 1, 5, 10, and 14 of a polyazaalkane. In some cases, spermine may have a nitrogen atom replacing a carbon atom at one or more of positions 1, 2, 3, 4, 5, 6, 7, 8, 9, 10, 11, 12, 13, or 14 of a polyazaalkane. In some cases, spermine may be charged or carry a charge. In some cases, a charged spermine or spermine carrying a charge may be spermine ion. In some cases, spermine ions may comprise spermine¹⁺, spermine²⁺, spermine³⁺, spermine⁴⁺, or a combination thereof. In some case, a buffer solution comprising spermine may also comprise a salt derivative of spermine.

In some cases, the buffer solution may comprise a concentration of up to about 1 nanomolar (nM), 5 nM, 10 nM, 100 nM, 500 nM, 1 micromolar (μM), 5 μM, 10 μM, 50 μM, 100 μM, 500 μM, 1 mM, 5 mM, 10 mM, 50 mM, 100 mM, 500 mM, 1 M, 5M, or 10 M of spermine. In some cases, the buffer solution may comprise a concentration of less than 1 nM of spermine. In some cases, the buffer solution may comprise a concentration of more than 10 M of spermine. In some cases, the buffer solution comprises a concentration within a range defined by any two of the preceding values. In some cases, the spermine comprises a salt derivative of spermine.

In some instances, the buffer solution may comprise an ion. In some cases, the buffer solution may comprise a cation. In some cases, an ion may comprise a divalent cation. Alternatively or in addition, an ion may comprise a monovalent cation, divalent cation, trivalent cation, quadrivalent cation, or pentavalent cation. In some cases, an ion may comprise magnesium ion. In some cases, an ion may comprise Mg²⁺. In some cases, the ion may comprise an ion of aluminum, barium, bismuth, cadmium, calcium, cesium, chromium, cobalt, copper, copper, hydrogen, iron, iron, lead, lithium, magnesium, mercury, mercury, nickel, potassium, rubidium, silver, sodium, strontium, or tin. In some cases, the buffer solution may comprise at least one of the ions of aluminum, barium, bismuth, cadmium, calcium, cesium, chromium, cobalt, copper, copper, hydrogen, iron, iron, lead, lithium, magnesium, mercury, mercury, nickel, potassium, rubidium, silver, sodium, strontium, or tin. In some cases, an ion may comprise Al³⁺, Ba²⁺, Bi³⁺, Cd²⁺, Ca¹⁺, Ca²⁺, Cs¹⁺, Cr³⁺, Co²⁺, Cu¹⁺, Cu²⁺, H¹⁺, Fe²⁺, Fe³⁺, Pb²⁺, Li¹⁺, Mg¹⁺, Mg²⁺, Hg2²⁺, Hg²⁺, Ni²⁺, K¹⁺, Rb¹⁺, Ag¹⁺, Na¹⁺, Sr²⁺, Sn²⁺, In some cases, the buffer solution may comprise at least one of Al³⁺, Ba²⁺, Bi³⁺, Cd²⁺, Ca¹⁺, Ca²⁺, Cs¹⁺, Cr³⁺, Co²⁺, Cu¹⁺, Cu²⁺, H¹⁺, Fe²⁺, Fe³⁺, Pb²⁺, Li¹⁺, Mg¹⁺, Mg²⁺, Hg2²⁺, Hg²⁺, Ni²⁺, K¹⁺, Rb¹⁺, Ag¹⁺, Na¹⁺, Sr²⁺, Sn². In some cases, the buffer solution may comprise a salt derivative of an ion. In some cases, the salt derivative of an ion may comprise a chloride salt of the ion.

In some instances, the buffer solution may comprise up to about 1 attomolar (aM), 5 aM, 8 aM, 10 aM, 50 aM, 80 aM, 100 aM, 500 aM, 800 aM, 1 femtomolar (fM), 5 fM, 8 fM, 10 fM, 50 fM, 80 fM, 100 fM, 500 fM, 800 fM, 1 picomolar (pM), 5 nM, 8 nM, 10 nM, 50 nM, 80 nM, 100 nM, 500 nM, 800 nM, 1 μM, 5 μM, 8 μM, 10 μM, 50 μM, 80 μM, 100 μM, 500 μM, 800 μM, 1 mM, 5 mM, 8 mM, 10 mM, 50 mM, 80 mM 100 mM, 500 mM, 800 mM, 1 M, 5 M, 8 M, or 10 M of an ion. In some cases, the buffer solution may comprise less than 1 aM of an ion. In some cases, the buffer solution may comprise more than 10M of an ion. In some cases, the buffer solution comprises a molarity of an ion within a range defined by any two of the preceding values. In some cases, the ion comprises a sodium ion or a salt derivative of a sodium ion. In some cases, a salt derivative of a sodium ion may comprise NaCl.

In some instances, the buffer solution may comprise Tris. In some cases. Tris may comprise tris(hydroxymethyl)aminomethane or tromethamine. In some cases. Tris in the buffer solution may have a concentration of about 20 mM (e.g., the buffer solution comprises about 20 mM Tris). In some cases, Tris in the buffer solution may have a concentration of up to about 10 nM, 50 nM, 100 nM, 500 nM, 1 μM, 5 μM, 10 μM, 50 μM, 100 μM, 500 μM, 1 mM, 5 mM, 10 mM, 50 mM, 100 mM, 500 mM. In some cases, Tris in the buffer solution may have a concentration of more than about 500 mM. In some cases, Tris in the buffer solution may have a concentration of less than about 10 nM. In some cases, Tris in the buffer solution may have a concentration that is within a range defined by any two of the preceding values.

In some instances, the buffer solution may comprise a concentration of up to about 1 aM, 5 aM, 10 aM, 50 aM, 50 aM, 100 aM, 500 aM, 1 fM, 5 fM, 10 fM, 50 fM, 100 fM, 500, fM, 1 pM, 5 pM, 10 pM, 50 pM, 100 pM, 500 pM, 1 nM, 5 nM, 10 nM, 50 nM, 100 nM, 500 nM, 1 μM, 5 μM, 10 μM, 50 μM, 100 μM, 500 μM, 1 mM, 5 mM, 10 mM, 50 mM, 100 mM, 500 mM. 1 M, 5 M, or 10 M of a sodium ion or a salt derivative of the sodium ion. In some cases, the buffer solution may comprise more than about 10M of a sodium ion or salt derivative of a sodium ion. In some cases, a salt derivative of a sodium ion may comprise NaCl.

In some instances, the buffer solution may decrease an average size (e.g., maximum dimension, such as a diameter) of at least a subset of a plurality of particles by up to about 1%, 5%, 10%, 15%, 20%, 25%, 30%, 35%, 40%, 45%, 50%, 55%, 60%, 65%, 70%, 75%, 80%, 85%, 90%, 95%, 99% or more (e.g., as compared with the average size of the particles prior to exposure to the buffer solution). In some cases, the buffer solution may decrease an average size of at least a subset of a plurality of particles by less than. In some cases, the buffer solution may decrease an average size of up to about 5%, 10%, 15%, 20%, 25%, 30%, 35%, 40%, 45%, 50%, 55%, 60%, 65%, 70%, 75%, 80%, 85%, 90%, 95%, 99% of particles in the plurality of particles.

In some instances, the method may further comprise washing the substrate of the buffer solution. In some cases, washing may be performed prior to, during, or subsequent to imaging. In some cases, washing the substrate of the buffer solution (e.g., upon or subsequent to a washing operation) may result in an average size of at least a subset of the plurality of particles returning to within up to about 5%, 10%, 15%, 20%, 25%, 30%, 35%, 40%, 45%, or 50% of the average size of the plurality of particles prior to adding the buffer solution. In some cases, upon or subsequent to a washing, an average size of at least a subset of a plurality of particles may be within at least about 5%, 10%, 15%, 20%, 25%, 30%, 35%, 40%, 45%, or 50% of the average size of the plurality of particles under conditions without washing.

The method may further comprise performing one or more operations on or with a plurality of particles on the substrate. In some cases, a method may comprise: (a) providing a substrate comprising a plurality of individually addressable locations, wherein a plurality of particles is immobilized to at least a subset of the plurality of individually addressable locations; (b) adding a buffer solution to a substrate, wherein the buffer solution decreases an average size of at least a subset of the plurality of particles; (c) subsequent to (b), imaging at least a portion of the substrate; (d) performing one or more operations on or with the plurality of particles on the substrate; (e) repeating (a); (f) repeating (b); and (g) repeating the imaging of (c). In some cases, a method may further comprise repeating a cycle of (d)-(g) up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some cases, an operation performed in (d) may comprise a nucleotide incorporation reaction with a nucleic acid molecule immobilized to a bead of a plurality of beads. In some cases, a nucleotide incorporation reaction may comprise or involve a reverse transcription, a nucleic acid extension, a nucleic acid amplification, a ligation, or a combination thereof. In some cases, imaging may comprise photometry.

In some instances, the plurality of particles comprises a plurality of sources that emit signals (e.g., fluorescent signals) during imaging. In some instances, the plurality of particles comprises a plurality of beads (e.g., sequencing beads as described herein). A bead may comprise a nucleic acid molecule immobilized thereto. In some cases, the nucleic acid molecule may comprise a fluorescent dye or other optically detectable moiety, e.g., coupled to the nucleic acid molecule during one or more operations (e.g., incorporation reactions). In some cases, a bead may comprise a plurality of nucleic acid molecules. Nucleic acid molecules of the plurality of nucleic acid molecules have sequence homology with each other. For example, a bead may comprise a colony of amplicons. In some instances, the plurality of particles comprises a plurality of DNA molecules (e.g., DNA molecules to be sequenced). In some instances, the plurality of DNA molecules have sequence complementarity. In some instances, a first subset of the plurality of DNA molecules have sequence complementarity and a second subset of the plurality of DNA molecules have sequence complementarity. In some instances, a particle in the plurality of particles comprises a plurality (e.g., a cluster) of DNA molecules, where the plurality of DNA molecules have sequence complementarity. In some instances, the plurality of particles comprises a plurality of DNA nanoballs.

In some instances, an average size of a plurality of particles, prior to, upon, or subsequent to the washing operation, is measured in Full-width at half-maximum (FWHM). As used herein, the term “FWHM” refers to a size (e.g., a diameter) of a particle determined from fluorescence imaging. In some instances. FWHM is the width of an intensity profile for the imaged particle, measured at the median intensity value (e.g., amplitude) detected from the particle (e.g., from an intensity profile of the fluorescence emitted from the particle). For instance, the FWHM may be determined for one or more particles in the plurality of particles, and an average size may be determined by averaging the one or more FWHM values so determined. In some instances, an intensity line profile corresponding to a respective particle is extracted from an image of the substrate. In some such instances, the FWHM for the particle is measured directly from the intensity line profile. In some such instances, the FWHM for the particle is estimated by fitting a Gaussian to the intensity line profile. In some instances, the FWHM for the particle is determined from a gray value version of the line intensity profile of the particle. In some instances, a FWHM may be determined for a particle at multiple time points (e.g., prior to, upon, and/or subsequent to a washing operation).

In some instances, average FWHM of a plurality of particles, may be up to about 1 nm, 5 nm, 10 nm, 50 nm, 0.1μ, 0.5 μm, 1 μm, 5 μm, 10 μm, 50 μm, 100 μm, 500 μm, 1 mm, or more. In some instances, an average size of a plurality of particles, prior to, upon, or subsequent to the washing operation, as measured in FWHM, may be up to about 1 nm, 5 nm, 10 nm, 50 nm. 0.1 μm, 0.5 μm, 1 μm, 5 μm, 10 μm, 50 μm, 100 μm, 500 μm, or 1 mm. In some instances, an average size of a plurality of particles prior to, upon, or subsequent to the washing operation, may be at least about 1 nm, 5 nm, 10 nm, 50 nm, 0.1 μm, 0.5 μm, 1 μm, 5 μm, 10 μm, 50 μm, 100 μm, 500 μm, 1 mm, or more. In some instances, an average size of a plurality of particles prior to, upon, or subsequent to the washing operation, may be greater than 1 mm. In some instances, an average size of a plurality of particles prior to, upon, or subsequent to the washing operation, may be less than 1 nm.

In some instances, the buffer solution may have a volume of less than about 1 mL, 0.9, 0.8 mL, 0.7 mL, 0.6 mL, 0.5 mL, 0.4 mL, 0.3 mL, 0.2 mL, 0.1 mL, 90 μL, 80 μL, 70 μL, 60 μL, 50 μL, 45 μL, 40 μL, 35 μL, 30 μL, 25 μL, 20 μL, 15 μL, 10 μL, or 5 μL.

In some instances, the buffer solution may comprise a plurality of particles mixed therein. In some cases, the plurality of particles may comprise at least about 10, 100, 1000, 10,000, 100,000, 1,000,000, 10,000,000, 100,000,000, 1,000,000,000, 10,000,000,000, 100,000,000,000 or more particles. In some cases, the plurality of particles may comprise at least about 100,000, 10,000,000, or 1,000,000,000 particles. In some cases, the plurality of particles may comprise at most about 100,000,000,000, 10,000,000,000, 1,000,000,000, 100,000,000, 10,000,000, 1,000,000, 100,000, 10,000, 1000, 100, or 10 particles. In some cases, the plurality of particles may comprise at most about 100,000, 10,000,000, or 1,000,000,000 particles.

In some instances, the plurality of particles in the buffer solution may comprise a concentration of up to about 10, about 100, about 1000, about 10,000, about 100,000, about 200,000, about 500,000, about 1,000,000, about 2,000,000, about 5,000,000, about 10,000,000, about 20,000,000, about 50,000,000, or about 100,000,000 particles per μL of the buffer solution. In some cases, the plurality of particles in the buffer solution may comprise a concentration of less than 10 particles per μL of the buffer solution. In some cases, the plurality of particles in the buffer solution may comprise a concentration of more than 100,000,000 particles per μL of the buffer solution. In some cases, the plurality of particles in the buffer solution may comprise a concentration within a range defined by any two of the preceding values.

In some instances, at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the independently addressable locations has at least one of a plurality of particles immobilized thereto.

Treating the particles with a buffer solution prior to imaging may decrease the average size or dimension of the particles, improving photometry quality during sequencing. This may be useful for various sequencing approaches, including flow-based sequencing, also referred to herein as a flow sequencing method.

a. Flow-Based Sequencing

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively.

Accordingly, the term “flow cycle order,” as used herein, generally refers to an ordering of flow cycles within the flow order and can be expressed in units of flow cycles. For example, where [A TGC] is identified as a 1^{st} flow cycle, and [A T G] is identified as a 2^{nd} flow cycle, the flow order of [A T G C A T G C A T G A T G A T G A T G C A T G C] may be described as having a flow-cycle order of [1^{st} flow cycle; 1^{st} flow cycle; 2^{nd} flow cycle; 2^{nd} flow; cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc.

Sequencing data can be generated using a flow sequencing method that includes extending a primer bound to a template molecule according to a pre-determined flow cycle where, in any given flow position, typically a single type of nucleotide is accessible to the extending primer. In some embodiments, at least some of the nucleotides of the particular type include a label, which upon incorporation of the labeled nucleotides into the extending primer renders a detectable signal. The resulting sequence by which such nucleotides are incorporated into the extended primer is expected to be the reverse complement of the sequence of the template polynucleotide molecule. Thus, an example method for generating sequencing data using a flow sequencing method comprises extending a primer using labeled nucleotides, detecting the presence or absence of a labeled nucleotide incorporated into the extending primer, and repeating these operations any number of times. Flow sequencing methods may also be referred to as “natural sequencing-by-synthesis.” “mostly natural sequencing-by-synthesis.” or “non-terminated sequencing-by-synthesis” methods. Exemplary methods are described in U.S. Pat. No. 8,772,473, which is incorporated herein by reference in its entirety.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having reversible terminators (e.g., 3′ reversible terminators), wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

Flow-based sequencing may be performed on colonies of amplified molecules, e.g., a plurality of beads with each bead representing one colony, where an optically resolvable location (e.g., individually addressable location) contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

The sequencing primer hybridized to the template molecule can be extended in a series of flow cycles, each flow cycle comprising one or more flow steps. After each flow, a signal indicative of the incorporation or lack thereof of the base(s) in the flow may be detected, such as by imaging the surface the polynucleotides are deposited on and analyzing the resulting image(s). Photometry may refer to imaging and/or processing of signals obtained from imaging, for example as described in PCT Patent App. No. PCT/US2022/074349, which is hereby incorporated by reference in its entirety. The signal obtained for any flow position in the sequencing data is flow-order-dependent in that the flow order used to sequence the template at any base position can affect the flow signal at that position. Prior to a subsequent flow, the extending primer-template hybrids may be subjected to a cleaving reagent to remove the labels that have been detected. At any point in time, the surface may be washed with a wash buffer.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Sequencing data, such as a flowgram as described below, can be generated. For example, Table 1 illustrates a flowgram generated for each of the example template sequences CTG, CAG, and CCG, which was sequenced by flow sequencing using a repeating flow cycle of T-A-C-G (that is, sequential addition of T, A, C, and G nucleotides). In Table 1, the value ‘l’ indicates incorporation of an introduced nucleotide, and the value ‘0’ indicates no incorporation of an introduced nucleotide, and an integer value ‘x’, where x>1, indicates incorporation of x introduced nucleotides. The flowgram can be used to determine the sequence of the template strand (e.g., the sequence of the template strand may be considered as the complement of the incorporated nucleotides).

TABLE 1

| | Flow Cycle | | | | | | | |
|---|---|---|---|---|---|---|---|---|
| | 1 | | | | 2 | | | |
| Cycle Step | 1 | 2 | 3 | 4 | 1 | 2 | 3 | 4 |
| Flow Bases | T | A | C | G | T | A | C | G |

Sequence | Number of Bases Incorporated

| | | | | | | | | |
|---|---|---|---|---|---|---|---|---|
| CTG | 0 | 0 | 0 | 1 | 0 | 1 | 1 | 0 |
| CAG | 0 | 0 | 0 | 1 | 1 | 0 | 1 | 0 |
| CCG | 0 | 0 | 0 | 2 | 0 | 0 | 1 | 0 |

A flowgram may be binary or non-binary. A binary flowgram detects the presence (1) or absence (0) of an incorporated nucleotide. A non-binary flowgram can also indicate the presence or absence of a base that is flowed in, but more quantitatively display a number of incorporated nucleotide (e.g., 2) from each stepwise introduction.

b. Treatment of Particles at Specific Flows to Improve Photometry

In flow-based sequencing where a plurality of particles is immobilized to a substrate during sequencing, a photometry workflow may comprise an initial particle detection operation to identify and index particle locations on the substrate, such as to build a particle catalogue. Such initial particle detection may be performed at or after the first flow or first set of flows in a flow order. Thereafter, for subsequent flows, the signals detected at such flows can be attributed to respective particles (or locations thereof) in the particle catalogue.

In some cases, the particles may be treated with the buffer solution at each imaging step in each flow. In some cases, the particles may be treated with the buffer solution at only at a subset of imaging steps of all the flows. In some cases, the particles may be treated with the buffer solution at only one or more specific imaging steps, such as the first imaging step, first two imaging steps, first three imaging steps, first four imaging steps, or any number of preamble imaging steps, of a sequencing flow order sufficient for one or more processors to process the signals to complete an initial detection of the particle locations on the substrate and build a particle catalogue: the particle catalogue can be referenced to attribute signals detected at subsequent imaging steps (for subsequent flows) to specific particles or locations thereof. In the latter instances, the particles need not be treated with the buffer solution in subsequent flows through the end of the sequencing run.

### Reducing Particle Aggregation and Improving Loading Efficiency

Sequencing (e.g., sequencing by synthesis) methods may comprise the use of particles (e.g., beads) coated with single-stranded nucleic acid molecules (e.g., ssDNA), such as single-stranded nucleic acid molecules primed with a PA-26 primer. Depending on the sequence, these single-stranded nucleic acid molecules may have significant secondary structure, which may be challenging to sequence through. Moreover, a single-stranded nucleic acid molecule coupled to a first particle may efficiently hybridize to single-stranded nucleic acid molecule coupled to a second particle if the sequences are compatible. Using human genomic (HG) DNA compounds this issue, as the human genome is highly repetitive, making it very likely that a given particle may encounter a compatible particle in a solution comprising a plurality of particles. If a particle does encounter a compatible particle, this interaction may be very stable and difficult to break up. Most importantly, such particle aggregates are impossible to sequence. Keeping particles separate is therefore paramount for high-throughput sequencing.

Disclosed herein are methods, systems, and compositions for reducing particle aggregation, increase substrate loading efficiency, and/or improving sequencing quality in nucleic acid sequencing processes. For example, the present disclosure provides methods, systems, and compositions for efficiently loading particles comprising nucleic acid molecules to be used in nucleic acid sequencing processes. Decreasing the average sizes of particles during loading can increase the number of particles that can be loaded onto a substrate for sequencing. Particles processed according to the methods, systems, and compositions provided herein may be useful in nucleic acid sequencing applications.

A method for processing a substrate (e.g., for sequencing) may comprise providing the substrate comprising a plurality of individually addressable locations, loading a plurality of particles onto the substrate (where particles in the plurality of particles comprise nucleic acid molecules), and subsequently adding a buffer solution to the substrate. In some cases, the method further comprises loading an additional plurality of particles onto the substrate. In some cases, a method for processing a substrate may comprise providing the substrate comprising a plurality of individually addressable locations and loading a plurality of particles onto the substrate concurrent with adding a buffer solution to the substrate. A plurality of particles may be immobilized to at least a subset of a plurality of individually addressable locations. The buffer solution may decrease an average size of at least a subset of the plurality of particles. In some instances, adding the buffer solution increases the percentage of individually addressable locations that have immobilized thereto particles from the plurality of particles (e.g., as compared with the percentage of individually addressable locations with particles immobilized thereto in the absence of adding the buffer solution). In some instances, the buffer solution comprises polyethylene glycol (PEG). In some instances, the buffer solution comprises spermine. In some instances, the buffer solution comprises MgCl₂. In some instances, the buffer solution comprises both PEG and MgCl₂.

The substrate may be any substrate described elsewhere herein. The plurality of particles may be any plurality of particles described elsewhere herein, such as a plurality of beads and/or a plurality of DNA nanoballs.

The method for processing a substrate may further comprise imaging at least a portion of the substrate during or subsequent to loading the plurality of particles onto the substrate. In some cases, imaging may detect at least about 100,000 particles/cm² of the substrate. In some cases, imaging may detect up to about 10,000, 50,000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,100,000, 1,200,000, 1,300,000, 1,400,000, 1,500,000, 1,600,000, 1,700,000, 1,800,000, 1,900,000, 2,000,000 particles/cm² of the substrate or more. In some cases, imaging may detect a number of particles/cm² of the substrate within a range defined by any two of the preceding values.

The imaging may detect a plurality of particles on the substrate. In some instances, imaging may detect up to about 10, 100, 1000, 10,000, 100,000, 1,000,000, 10,000,000, 100,000,000, 1,000,000,000, 10,000,000,000, 100,000,000,000 or more particles. In some cases, imaging may detect more than about 100,000,000,000 particles. In some cases, imaging may detect less than 10 particles. In some instances, imaging may detect at least a subset of a plurality of particles on the substrate. In some cases, imaging may detect up to about 5%, 10%, 15%, 20%, 25%, 30%, 35%, 40%, 45%, 50%, 55%, 60%, 65%, 70%, 75%, 80%, 85%, 90%, 95%, 99%, or more of a plurality of particles. In some cases, imaging may detect less than 5% of a plurality of particles. In some cases, imaging may detect more than 99% of a plurality of particles.

The buffer solution may be any buffer solution described elsewhere herein, for example the buffer solution described with respect to improving photometry.

In some instances, the buffer solution may decrease an average size (e.g., maximum dimension, such as a diameter) of at least a subset of a plurality of particles by up to about 1%, 5%, 10%, 15%, 20%, 25%, 30%, 35%, 40%, 45%, 50%, 55%, 60%, 65%, 70%, 75%, 80%, 85%, 90%, 95%, 99% or more. In some cases, the buffer solution may decrease an average size of at least a subset of a plurality of particles by less than. In some cases, the buffer solution may decrease an average size of up to about 5%, 10%, 15%, 20%, 25%, 30%, 35%, 40%, 45%, 50%, 55%, 60%, 65%, 70%, 75%, 80%, 85%, 90%, 95%, 99% of particles in the plurality of particles. In some instances, the method may further comprise washing the substrate of the buffer solution. In some cases, washing may be performed prior to, during, or subsequent to imaging.

The method may further comprise performing one or more operations on or with a plurality of particles on the substrate. In some cases, a method may comprise: (a) providing a substrate comprising a plurality of individually addressable locations, wherein a plurality of particles is immobilized to at least a subset of the plurality of individually addressable locations; (b) adding a buffer solution to a substrate, wherein the buffer solution decreases an average size of at least a subset of the plurality of particles; (c) subsequent to (b), imaging at least a portion of the substrate; (d) performing one or more operations on or with the plurality of particles on the substrate; (e) repeating (a); (f) repeating (b); and (g) repeating the imaging of (c). In some cases, a method may further comprise repeating a cycle of (d)-(g) up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some cases, an operation may comprise a nucleotide incorporation reaction with a nucleic acid molecule immobilized to a bead of a plurality of beads. In some cases, a nucleotide incorporation reaction may comprise or involve a reverse transcription, a nucleic acid extension, a nucleic acid amplification, a ligation, or a combination thereof. In some cases, imaging may comprise photometry.

In some instances, an average size of a plurality of particles, upon incubation with the buffer solution is measured in Full-width at half-maximum (FWHM).

In some instances, average FWHM of the plurality of particles, may be up to about 1 nm. 5 nm, 10 nm, 50 nm, 0.1 μm, 0.5 μm, 1 μm, 5 μm, 10 μm, 50 μm, 100 μm, 500 μm, 1 mm, or more. In some instances, an average size of the plurality of particles, prior to, upon, or subsequent to the washing operation, as measured in FWHM, may be up to about 1 nm, 5 nm, 10 nm, 50 nm, 0.1 μm, 0.5 μm, 1 μm, 5 μm, 10 μm, 50 μm, 100 μm, 500 μm, or 1 mm. In some instances, an average size of the plurality of particles prior to, upon, or subsequent to the washing operation may be at least about 1 nm, 5 nm, 10 nm, 50 nm, 0.1 μm, 0.5 μm, 1 μm, 5 μm, 10 μm, 50 μm, 100 μm, 500 μm, 1 mm, or more. In some instances, an average size of a plurality of particles prior to, upon, or subsequent to the washing operation may be greater than 1 mm. In some cases, an average size of a plurality of particles prior to, upon, or subsequent to the washing operation may be less than 1 nm.

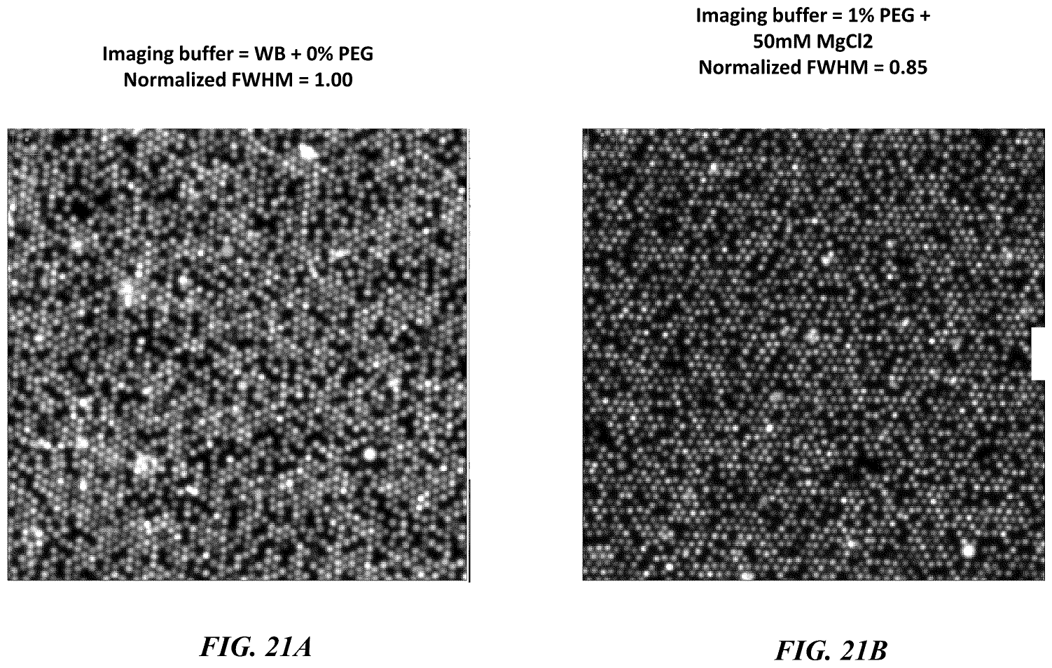

An exemplary image of a plurality of particles immobilized to a substrate is shown in FIG. 21A, where the plurality of particles is imaged in a buffer that lacks any PEG. In comparison, an exemplary image of a plurality of particles immobilized to a substrate and imaged in a buffer comprising 1% w/v PEG and 50 mM MgCl₂ is shown in FIG. 21B. The FWHM for FIGS. 21A and 21B are normalized to the FWHM measured for FIG. 21A. Using a buffer containing PEG and a divalent cation decreases the measured FWHM by approximately 15% compared with using a buffer that lacks both PEG and a divalent cation.

In some embodiments, one or more of a temperature, an incubation time, a surfactant, a polymer concentration, or a salt concentration of a solution (e.g., a buffer) comprising beads may be adjusted to increase bead occupancy or to increase bead loading efficiency. In some embodiments, one or more of a temperature, an incubation time, a surfactant, a polymer concentration, or a salt concentration of a solution (e.g., a buffer) on a substrate (e.g., a wafer) may be adjusted to increase bead occupancy or to increase bead loading efficiency on the substrate.

A solution may be incubated on the substrate. In some embodiments, the solution may be incubated on the substrate under conditions that maintain a layer of fluid on the surface. The solution may be incubated for at least about 5, 10, 15, 20, 25, 30, 35, 40, 45, 50, 55, 60, 65, 70, 75, 80, 85, or 90 minutes. In some cases the incubation time may be within a range defined by any two of the preceding values. In some cases, the incubation may be for more than 90 minutes. In some instances, the layer of fluid may be maintained at a film thickness of at least 10 nm, 20 nm, 50 nm, 100 nm, 200 nm, 500 nm, 1 μm, 2 μm, 5 μm, 10 μm, 20 μm, 50 μm, 100 μm, 200 μm, 500 μm, or 1 mm during incubation. One or more of the temperature of the chamber, the humidity of the chamber, the rotation of the substrate, or the composition of the fluid (e.g., the solution) may be adjusted such that the layer of fluid is maintained on the substrate during incubation. It is advantageous to maintain a minimum threshold fluid layer to facilitate bead dispersion and settling onto individually addressable locations (e.g., reducing aggregation while promoting high bead occupancy).

In some embodiments, a buffer solution used during dispensing a plurality of particles onto the substrate has a first concentration of a polymer (e.g., PEG) and a first concentration of a salt (e.g., magnesium chloride). In some embodiments, prior to dispensing a plurality of particles onto the substrate, the substrate is incubated in the presence of another buffer solution, wherein the other buffer solution has a second concentration of the polymer and a second concentration of the salt. In some embodiments, the second concentration of the polymer is greater than the first concentration of the polymer. In some embodiments, the second concentration of the salt is greater than the first concentration of the salt.

In some embodiments, a method is provided with a first plurality of particles immobilized thereto at a first density of particles per total substrate surface area. The method proceeds by dispensing a buffer solution to the substrate, where an average maximum dimension of the first plurality of particles decreases subsequent to contacting the buffer solution. The method continues by dispensing a second plurality of particles onto the substrate, to immobilize at least a subset of the second plurality of particles on the substrate. This yields a particle-loaded substrate having a second density of particles per total substrate surface area, where the second density is higher than the first density.

### EXAMPLES

These examples are provided for illustrative purposes only and are not intended to limit the scope of the claims provided herein.

### Example 1: Shrinking Beads Using a Shrinkage Buffer During Imaging

Various polymers or cations were tested for providing better resolution of individual particles during imaging. Particles, e.g., ISP-5HG beads (ThermoFisher's Ion sphere particles (ISPs)), were prepared (e.g., attached or annealed to) DNA molecules amplified by emulsion PCR (emPCR). The beads were dispensed onto wafers for imaging, where the dispensing was performed using typical loading buffers. Prior to imaging, wafers were exposed to buffers comprising one or more of the various polymers or cations to be tested. The size of beads (e.g., FWHM) was determined by using a detector during imaging. Specifically, the bead sizes were determined from fluorescent imaging.

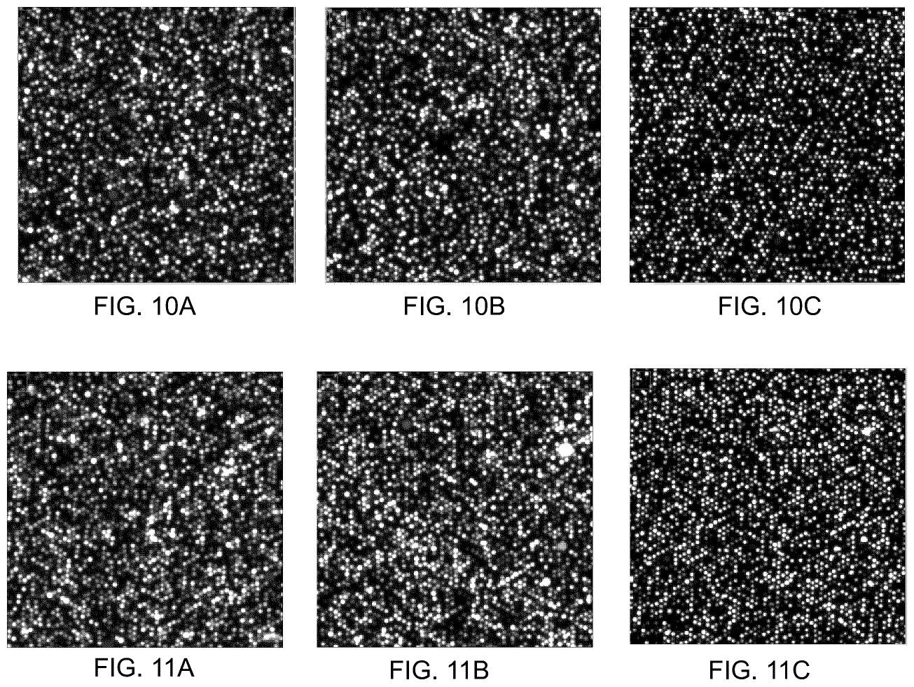

The sizes of the beads in the presence of a shrinkage buffer (20 mM Tris, 80 mM NaCl. 0.05% w/v Triton X-100, 10% w/v PEG-4000, and 50 mM Mg²⁺ (FIG. 10C) were smaller than those the presence of a control imaging buffer (20 mM Tris, 80 mM NaCl, and TritonX0.05% w/v Triton X-100) (FIG. 10A) or a shrinkage buffer without PEG (20 mM Tris, 80 mM NaCl. and TritonX0.05% w/v Triton X-100 and 50 mM Mg²⁺) (FIG. 10B). Similar shrinkage of the beads was also observed in the presence of PEG-8000 (e.g., in a shrinkage buffer comprising 20) mM Tris, 80 mM NaCl. 0.05% w/v Triton X-100, 10% w/v PEG-8000, and 50 mM Mg²⁺). The sizes of the beads in the image taken in the presence of a shrinkage buffer comprising PEG-8000 (see FIG. 11C) were smaller than those taken in the presence of the control imaging buffer (20 mM Tris, 80 mM NaCl. TritonX) (FIG. 11A) or the shrinkage buffer without PEG (FIG. 11B). The Mg²⁺ present in the different shrinkage buffers was derived from MgCl₂

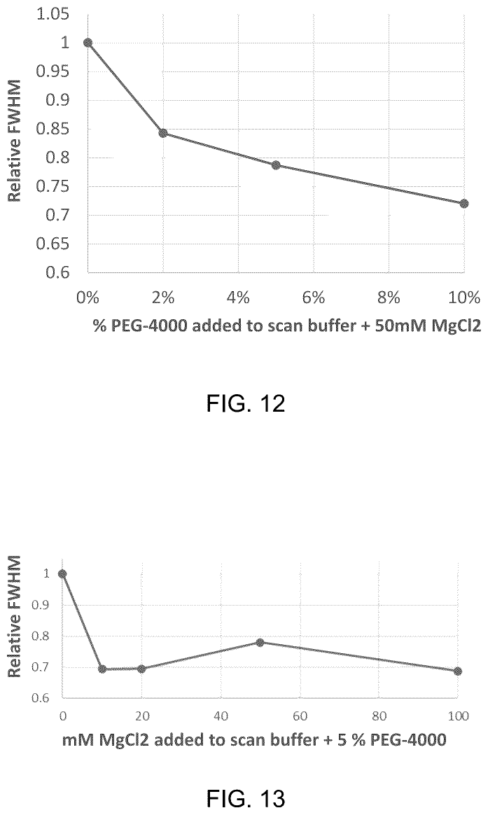

The average FWHM of beads in the first imaging buffer (20 mM Tris, 80 mM NaCl. TritonX) was measured at 1.11 μm. In contrast, the average FWHM of beads in the presence of the fifth imaging buffer (20 mM Tris, 80 mM NaCl. TritonX. 10% w/v PEG-8000, 50 mM Mg²⁺) was measured at 0.82 μm, about 30% (1.11 μm) smaller than that imaged under the control imaging buffer. The totality of this data suggests that both polymers (e.g., PEG) and cations (e.g., Mg²⁺) can decrease the size of the bead when added in the imaging buffer. For example, as shown in FIG. 12, increasing the amount of PEG in the shrinkage imaging buffer (20 mM Tris. 80 mM NaCl. TritonX. 50 mM Mg²⁺) decreased the average size of the beads. 10% PEG could decrease the average bead size by greater than 25%. Furthermore, as shown in FIG. 13, concentrations as low as 10 mM Mg²⁺ added to the shrinkage imaging buffer (20 mM Tris 80 mM NaCl, TritonX, 10% w/v PEG) could decrease the size of the beads by about 30%. Concentrations of Mg²⁺ lower than 10 mM Mg²⁺ are also expected to decrease the sizes of the beads when used in the imaging buffer.

Increasingly more particles can be resolved (e.g., detecting using an imaging system such as system 800 as described above) in cases where the average size of the particles is decreased. As shown in Table 2, significantly (p value<<<0.001) more beads could be detected and resolved when beads were shrunk by the shrinkage buffer (e.g., the imaging buffer with the addition of one or more shrinking agents as described herein: Tris, 80 mM NaCl, TritonX, 10% w/v PEG-4000, and 50 mM Mg²⁺), as opposed those of the control buffer (e.g., the imaging buffer that comprises Tris, 80 mM NaCl, and TritonX). Table 2 illustrates data collected from multiple locations on a substrate (e.g., from imaging tiles of equal area).

TABLE 2
Numbers of Beads Detected in Different Imaging Buffers

| | Number of beads | Number of beads | Percentage increase |
|---|---|---|---|
| Experimental | detected in the | detected in the | in number of beads |
| Tile | control buffer | shrinkage buffer | detected |
| 1 | 142487 | 160596 | 12.7% |
| 2 | 152283 | 163880 | 7.6% |
| 3 | 147408 | 169378 | 14.9% |
| 4 | 147726 | 164038 | 11.0% |
| 5 | 149321 | 164719 | 10.3% |
| 6 | 149567 | 170649 | 14.1% |
| 7 | 148315 | 163605 | 10.3% |
| 8 | 148531 | 164428 | 10.7% |
| 9 | 152761 | 165410 | 8.3% |
| 10 | 146357 | 164293 | 12.3% |
| 11 | 149975 | 163931 | 9.3% |
| 12 | 148710 | 167024 | 12.3% |
| 13 | 137628 | 163411 | 18.7% |
| 14 | 144214 | 163573 | 13.4% |
| 15 | 147977 | 160262 | 8.3% |
| 16 | 150069 | 167272 | 11.5% |

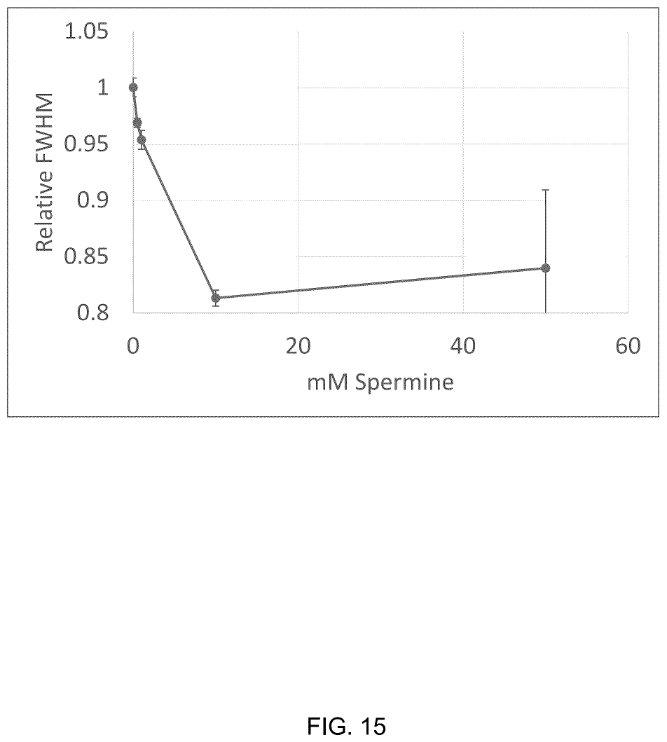

In some instances, other molecules can replace either PEG or Mg²⁺ in the shrinkage buffer. For example, cations such as Ca²⁺ may be used in place of Mg²⁺. Furthermore, as shown in FIG. 15, spermine alone, which can be positively charged as monovalent, divalent, trivalent, or tetravalent ions, could also shrink the size of the beads when added to the shrinkage buffer, even at concentrations as low as 10 mM. These data suggest that different polymers and cations when used in the imaging buffer can decrease the size of the particles during imaging, thus facilitating the resolution of the individual particles on a substrate (e.g., during fluorescence imaging).

In many cases, beads comprise polymer-based structures and contain a large amount of water. PEG is predicted to function as a thickening agent (due to its structure as a large inert polymer with a branching structure) and helps shrink beads by removing water from the beads. Mg²⁺ is predicted to interact with polynucleotides (e.g., nucleic acid molecules immobilized to a bead) and reduce electrostatic force (e.g., the compression of negatively-charged polynucleotides as beads shrink in diameter).

### Example 2: Bead Shrinkage is Maintained Over Multiple Imaging Cycles

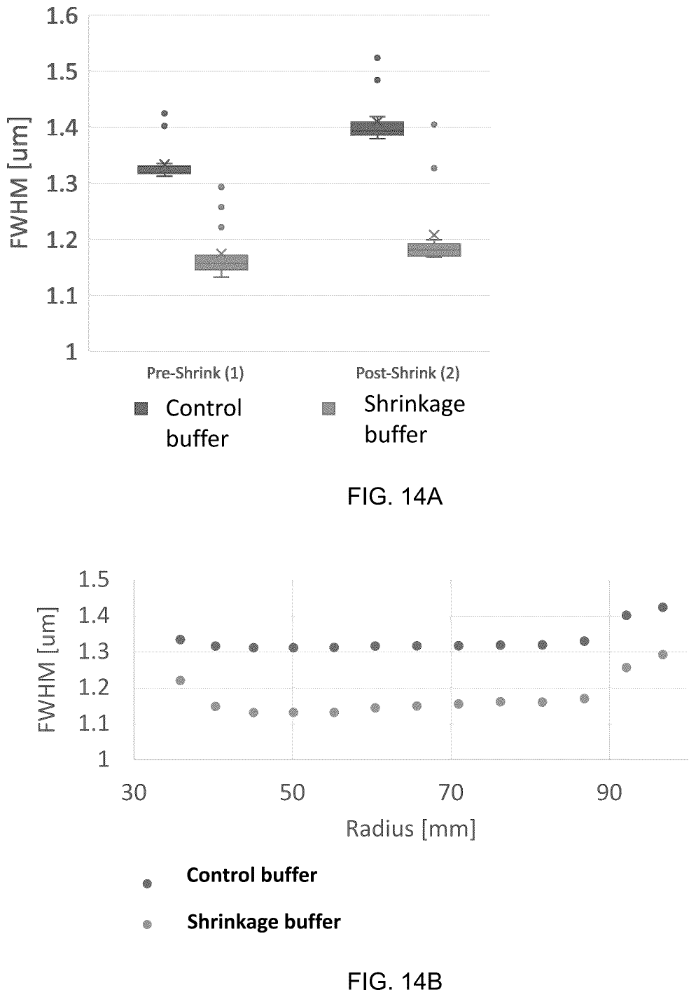

An experiment was conducted to assess whether the decrease of the size of the beads in the presence of a shrinkage buffer can be maintained over multiple imaging cycles. The ISP-HG beads ePCR5130 were prepared by attaching DNAs amplified by emulsion PCR (emPCR). The beads were loaded onto a wafer for imaging. In imaging cycle #1, the beads were first imaged in the presence of an imaging buffer. Approximately 20 mL of imaging buffer were pipetted onto the wafer such that it was evenly dispersed to cover the whole wafer. The wafer was incubated for 60 seconds, washed briefly, and imaged in the imaging cycle #2. The wafer was washed and imaged again in the imaging cycle #3. As shown in FIG. 14A, when a shrinkage buffer (20 mM Tris, 80 mM NaCl. TritonX. 10% w/v PEG-4000, 50 mM Mg²⁺) was used as the imaging buffer, the sizes of the beads were measured to be approximately 10-20% smaller, compared to bead sizes measured in the control imaging buffer (20 mM Tris, 80 mM NaCl. TritonX). These data indicate that shrinkage of the beads can be maintained over multiple imaging cycles when a shrinkage buffer is used during imaging. In some cases, the shrinkage buffer may be washed out of the wafer concurrent with or subsequent to imaging.

To determine if the bead shrinkage by the shrinkage buffer could be maintained over the whole substrate surface, the sizes of the beads detected in different imaging buffers were compared across different radii of the wafer. As shown in FIG. 14B, the sizes of the beads imaged with the shrinkage buffer were smaller than those imaged with the control buffer across the whole wafer. Therefore, bead shrinkage in the presence of the shrinkage buffer can be maintained over multiple imaging cycles across the whole surface of the wafer.

### Example 3: Bead Shrinkage is at Least Partially Reversible

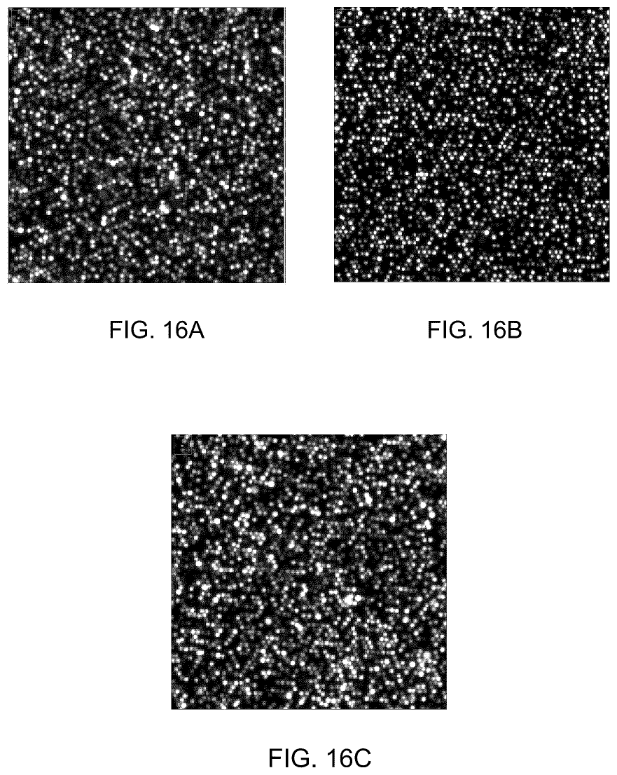

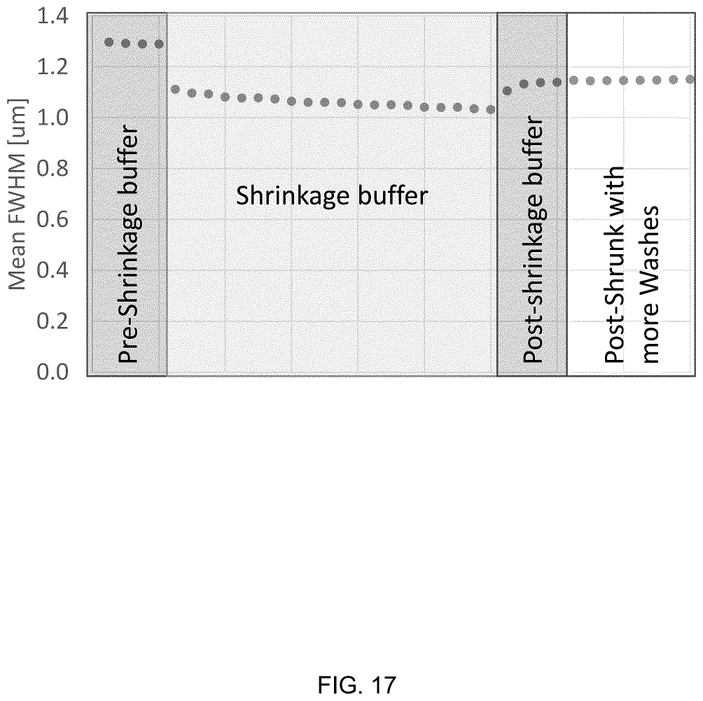

Because bead shrinkage can potentially affect the chemistry of the beads, the reversibility of the bead shrinkage was tested. ISP-5HG beads (ThermoFisher's Ion sphere particles (ISPs)) were prepared by attaching DNAs amplified by emulsion PCR (emPCR). The beads were dispensed onto wafer for imaging in the presence of a control buffer (20 mM Tris, 80 mM NaCl. TritonX) (FIG. 16A). After the imaging, the wafer was washed thoroughly, and the beads were imaged in the presence of a shrinkage buffer (20 mM Tris, 80 mM NaCl. TritonX. 10% w/v PEG-8000, 50 mM Mg²⁺) (FIG. 16B). The beads were washed again and imaged in the presence of the control buffer (FIG. 16C). As shown in FIGS. 16A-16C, the size of the beads decreased in the shrinkage buffer and increased in the control buffer. Consistently, when the average sizes of the beads (FWHM) imaged in the presence of the control buffer and a different shrinkage buffer (20) mM Tris, 80 mM NaCl, TritonX, 10% w/v PEG-4000, 50 mM Mg²⁺) were compared in FIG. 17, the bead showed shrinkage under the shrinkage buffer. When the control buffer replaced the shrinkage buffer, the size of the beads increased, albeit not completely. Hence, the bead shrinkage is at least partially reversible and is not expected to affect the physical chemistry of the beads (and/or the performance of the beads e.g., as substrates for sequencing reactions).

### Example 4: Increasing Surface Bead Occupancy via Repeated Bead Loading

A substrate, such as those described herein, may have a large number of individually addressable locations, where each such individually addressable location is a potential site to immobilize a particle comprising one or more nucleic acid molecules. The efficiency of loading (e.g., the percentage of a substrate's individually addressable locations that are occupied by a sequencing particle) impacts the efficiency of nucleic acid molecule sequencing that is possible (e.g., in subsequent sequencing reactions). Methods for increasing the loading efficiency of a substrate by performing successive particle (e.g., bead) loadings were tested.

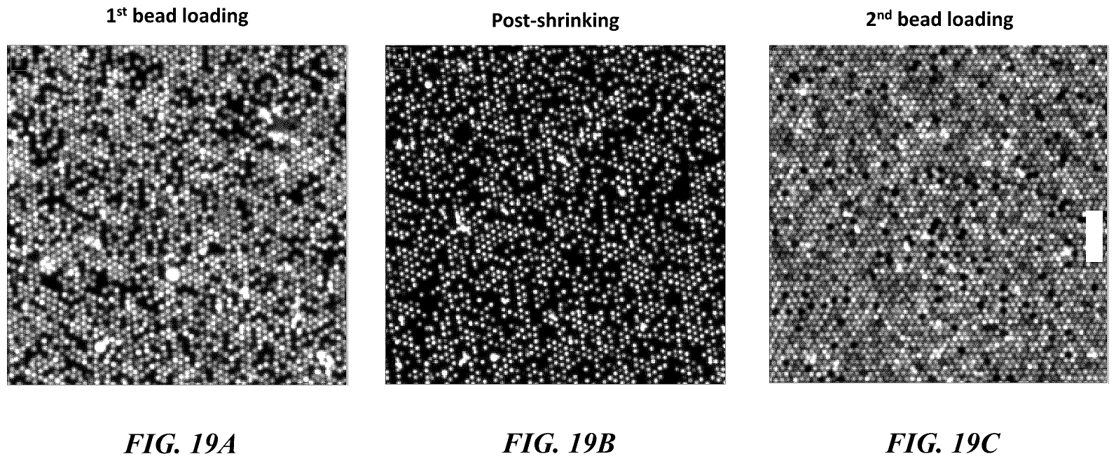

Particles, e.g., ISP-5HG beads (ThermoFisher's Ion sphere particles (ISPs)), were prepared (e.g., attached or annealed to) DNA molecules amplified (e.g., by emulsion PCR). The beads were dispensed onto substrates (e.g., wafers or wafer coupons) for imaging. The dispensing was performed using typical loading buffers (e.g., buffers lacking a polymer, such as PEG). An exemplary image of the results of the first dispense of beads is shown in FIG. 19A. After the first bead dispense, wafers were exposed to shrinkage buffer comprising polymers (e.g., 10% w/v PEG-8000). An exemplary image of the results of the applying a shrinking buffer is shown in FIG. 19B. After exposure to shrinkage buffer, additional beads were dispensed onto the wafers. An exemplary image of the results of the second bead dispense are shown in FIG. 19C. Each dispense step comprised incubation of beads on the wafer for approximately 60 minutes.

After each step-first bead dispense, shrinkage, and second bead dispense—the percent occupancy (e.g., average % occupancy) of the wafer was determined. Example occupancy measurements are shown in Table 3. Wafer occupancy was determined from the number of beads immobilized (e.g. to individually addressable locations) in an area of the substrate/the possible number of individually addressable locations in the area of the substrate. The number of possible individually addressable locations is proportional to the pitch (e.g., distance of the center of one individually addressable location to a next closest individually addressable location) of individually addressable locations on the wafer or wafer coupon. The experiments detailed in Table 3 were performed on wafer coupons with 1.6 μm pitch (e.g., there are on average 1.6 μm between an individually addressable location and a next closest individually addressable location. Similar results would be expected from performing the first bead dispense, shrinking, and second bead dispense on a wafer with varying pitches.

TABLE 3
Resulting occupancy of multiple bead
loadings on 1.6 um substrate coupons

| Experiment | Step | Average % occupancy |
|---|---|---|
| A | 1^{st} bead loading | 78% |
| | post-shrinking | 79% |
| | 2^{nd} bead loading | 89% |
| B | 1^{st} bead loading | 83% |
| | post-shrinking | 83% |
| | 2^{nd} bead loading | 95% |

Table 3 shows the results from 2 independent experiments, A and B. In both experiments, the 2^{nd} bead loading resulted in higher average percent occupancy (e.g., 89% and 95%, respectively) than that observed after the 1st bead loading (e.g., 78% and 83%, respectively). This indicates that whatever the initial loading efficiency, multiple loading steps can be successful at immobilizing an additional plurality of beads to individually addressable locations on the surface. As an internal control, in each experiment, the average percentage occupancy observed after adding shrinkage buffer did not vary significantly from the average percentage occupancy observed after the first loading (e.g., indicating that the addition of the shrinkage buffer did not displace any beads in the first plurality of beads that had immobilized to individually addressable locations prior to the second bead loading step).

Additional tests were performed comparing shrinkage buffers comprising different molecular weights of PEG. See Table 4. PEG-8000 has a higher viscosity than PEG-4000 (or other smaller molecular weight PEGs). As shown in Table 4, which illustrates duplicate experiments of each scenario (e.g., independent experiments A and B with 10% w/v PEG-8000, and independent experiments C and D with 10% w/v PEG 4000), average percent occupancy generally increased when PEG-4000 was used as compared to the use of PEG-8000. For example, in experiment A the average percent occupancy (e.g., of a wafer coupon) after the first bead loading step was 78% and increased to 89% after incubating with the shrinking buffer and performing the second bead loading step. Experiment C illustrated even higher occupancies, where the average percent occupancy was 89% after the first bead loading step and increased to 95% after incubating with the shrinking buffer and performing the second bead loading step. This demonstrates that the use of a less viscous PEG molecule (e.g., a lower molecular weight PEG) is useful in increasing bead occupancy on a wafer (note, in both experiments bead occupancy increased as a result of the second bead loading step).

TABLE 4
Bead occupancy with respect to buffer viscosity

| Shrinking | | | Average % |
|---|---|---|---|
| Agent | Experiment | Step | occupancy |
| 10% PEG- | A | 1^{st} bead loading | 78% |
| 8000 | | shrinking buffer + 2^{nd} bead | 89% |
| | | loading | |
| | B | 1^{st} bead loading | 83% |
| | | shrinking buffer + 2^{nd} bead | 95% |
| | | loading | |
| 10% PEG- | C | 1^{st} bead loading | 89% |
| 4000 | | shrinking buffer + 2^{nd} bead | 95% |
| | | loading | |
| | D | 1^{st} bead loading | 83% |
| | | shrinking buffer + 2^{nd} bead | 91% |
| | | loading | |

In Table 4 the substrate (e.g., the wafer coupon) was incubated for 1 hour for each of the first and second bead loading steps. Decreasing the amount of time required for such incubations would improve the overall efficiency of the bead loading process. Table 5 shows the results of incubating the substrate for both bead loading steps for 30 minutes versus 60 minutes. As seen by comparing experiments A and B (30 minute incubation) with experiments C and D (60 minute incubation), longer incubation times are correlated with higher average bead loading occupancies (e.g., 73% and 79% versus 95% and 91%, respectively). Longer incubation times are preferred as this provides beads more opportunities to immobilize to individually addressable locations on the substrate.

TABLE 5
Bead occupancy with respect to incubation time

| Incubation Time | Experi- | | Average % |
|---|---|---|---|
| per Loading | ment | Step | occupancy |
| 30 minutes | A | 1^{st} bead loading | 66% |
| | | shrinking buffer + 2^{nd} bead | 73% |
| | | loading | |
| | B | 1^{st} bead loading | 66% |
| | | shrinking buffer + 2^{nd} bead | 79% |
| | | loading | |
| 60 minutes | C | 1^{st} bead loading | 86% |
| | | shrinking buffer + 2^{nd} bead | 95% |
| | | loading | |
| | D | 1^{st} bead loading | 86% |
| | | shrinking buffer + 2^{nd} bead | 91% |
| | | loading | |

In some instances, more than 1, 2, 3, 4, 5, 6, 7, 8, 9, or 10 bead dispense steps may be performed. In some instances, each bead dispense step may comprise a same volume. In some instances, each bead dispense step may comprise a same number of beads (and/or concentration of beads). In some instances, each bead dispense step may be performed with a same buffer (e.g., either during loading and/or during pre-loading incubation). In some instances, at least one bead dispense step may comprise a different volume. In some instances, at least one bead dispense step may comprise a different number of beads (and/or concentration of beads). In some instances, at least one bead dispense step may be performed with a different buffer (e.g., without shrinking agents such as PEG and/or Mg²⁺).

### Example 5: Increasing Surface Bead Occupancy Via Single Bead Loading

An additional method of applying shrinking buffer to beads (e.g., for the purpose of increasing loading efficiency) was performed. In Example 4, multiple rounds of bead loading were performed to achieve higher loading efficiencies. The use of shrinking agents during loading, as opposed to during a separate shrinking step, would help improve the efficiency of bead loading, and subsequent sequencing reactions. As shown in Table 6, an increase the percentage of PEG spiked into loading buffer (e.g., the percentage of PEG-8000 in loading buffer) is correlated with an increase in the average percent occupancy of beads on the substrate. Alternative weights of PEG (e.g., PEG-4000, PEG-400, etc.) are expected to have similar effects (e.g., as found elsewhere herein in other experiments).

TABLE 6
Bead occupancy when shrinking buffer is used
during loading at various percentages

| % PEG8000 in loading buffer | Average % occupancy |
|---|---|
| 0% | 78-83% |
| 0.01% | 87% |
| 0.1% | 84% |
| 0.5% | 91% |
| 0.5% | 95% |
| 1.0% | 93% |
| 1.0% | 93% |

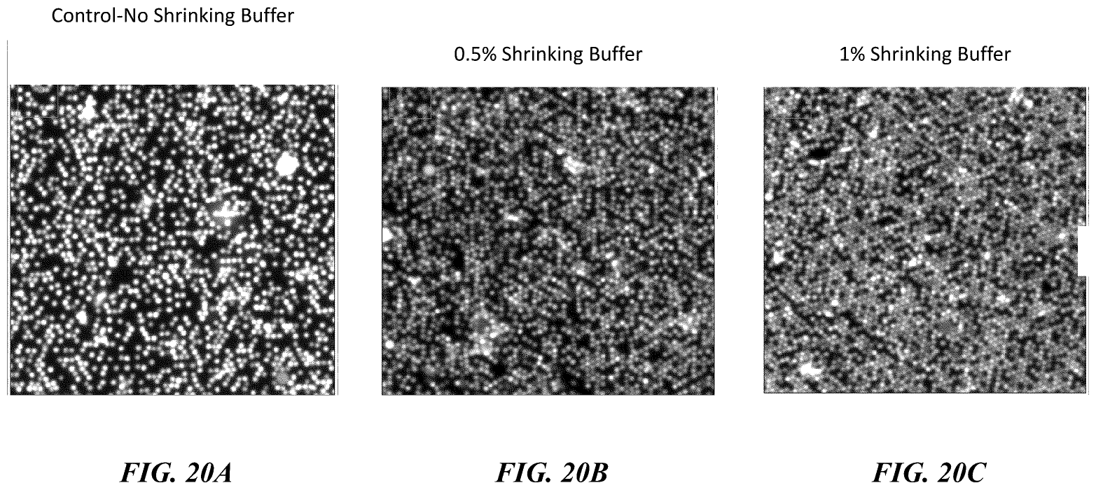

Exemplary images of bead loading as shown in FIGS. 20A-20C. Various percentages of PEG-8000 were present in the buffer used for bead loading, on wafers with 1.4 μm pitch. In FIG. 20A, 0% w/v PEG-8000 was present in the loading buffer, resulting in an average percent occupancy of 48%. In FIG. 20B, 0.5% w/v PEG-8000 was present in the loading buffer, resulting in an average percent occupancy of 53%. In FIG. 20C, 1% w/v PEG-8000 was present in the loading buffer, resulting in an average percent occupancy of 67%. This confirms the results illustrated in Table 6, where an increase in the percentage of PEG aids in improving wafer occupancy.

### Example 6: Surface Bead Occupancy on Different Pitch Sizes

The efficacy of bead loading with respect to pitch size was tested. Following Example 5, a shrinking buffer was used during bead loading (e.g., to reduce an effective dimension of the beads during bead loading). As seen in Table 7 in these independent experiments, regardless of whether the pitch size is 1.5 μm or 1.4 μm, the average percent occupancy of the substrate (e.g., the percentage of available individually addressable locations with a sequencing particle immobilized thereto) increased as the percentage of shrinking buffer used during loading increased.

TABLE 7
Bead occupancy with respect to pitch size

| | | | Average % |
|---|---|---|---|
| Pitch size | Experiment | Condition | occupancy |
| 1.5 μm | A | Control | 71% |
| | | 0.5% Shrinking Buffer | 78% |
| | | 1% Shrinking Buffer | 85% |
| | B | Control | 75% |
| | | 0.5% Shrinking Buffer | 67% |
| | | 1% Shrinking Buffer | 84% |
| | C | Control | 71% |
| | | 0.5% Shrinking Buffer | 78% |
| | | 1% Shrinking Buffer | 85% |
| | D | Control | 57% |
| | | 0.5% Shrinking Buffer | 60% |
| | | 1% Shrinking Buffer | 65% |
| | | 1.5% Shrinking Buffer | 68% |
| 1.4 μm | E | Control | 48% |
| | | 0.5% Shrinking Buffer | 53% |
| | | 1% Shrinking Buffer | 67% |
| | F | Control | 45% |
| | | 0.5% Shrinking Buffer | 62% |
| | | 1% Shrinking Buffer | 60% |
| | | 1.5% Shrinking Buffer | 71% |

This confirms the results detailed in Example 5. The control buffers comprised 10 mM Tris pH 7, 0.05% Tergitol and 50 mM MgCl₂. The 0.5% shrinking buffer comprised 0.5% w/v PEG-8000, while the 1% shrinking buffer comprised 1% w/v PEG-8000. In some cases, the substrate may be further incubated with a buffer comprising a divalent cation prior to dispensing beads onto the substrate (e.g., as described in Example 8).

### Example 7: Prolonged Exposure to Mg²⁺ Encourages Bead Aggregation

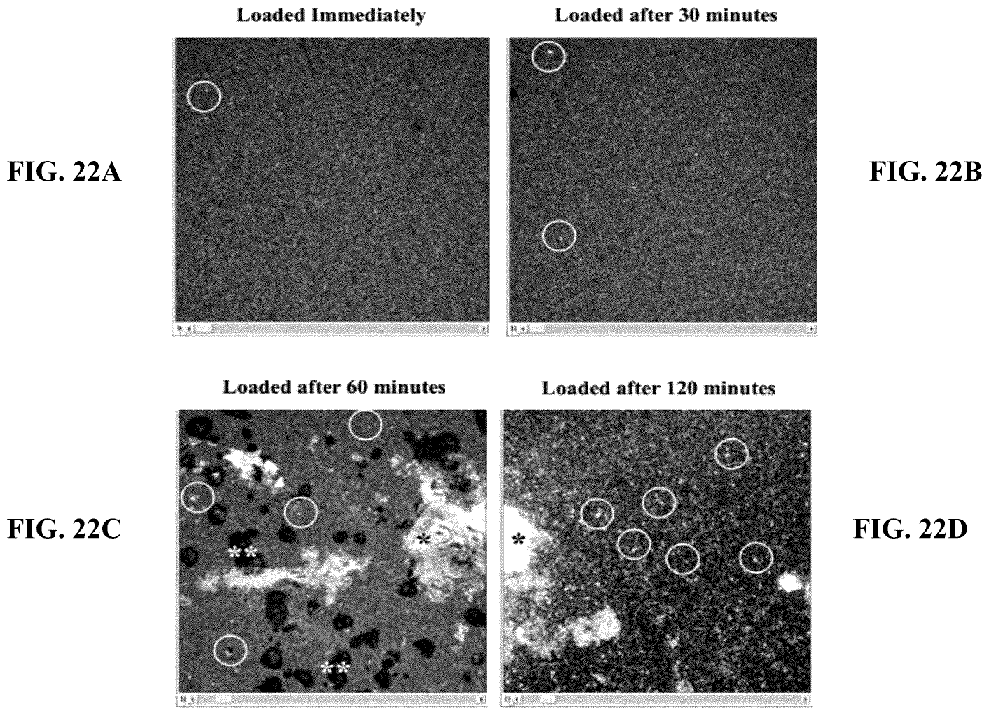

As described herein, prolonged exposure of a plurality of beads to cations, such as divalent cations Mg²⁺ or Ca²⁺, encourages bead aggregation. To identify a role of cations in bead aggregation, beads (ThermoFisher's Ion sphere particles (ISPs)) prepared by attaching DNAs amplified by emulsion PCR (emPCR) were incubated with a TTM buffer (10 mM of Tris, pH=7.0; 0.055% Tergitol: 10 mM MgCl₂) for various amount of times (0 minutes, 30 minutes, 60 minutes, and 120 minutes) in a sample tube before dispensing the beads onto wafers. As shown in FIGS. 22A-22D, which show coupon wafers (e.g., sections of wafers) of loaded beads, loading the beads to the wafer immediately after incubation with the TTM buffer did not cause significant bead aggregation (e.g., in FIG. 22A). However, the numbers of bead aggregates on the wafer increased with the time the beads were incubated in the TTM buffer, as indicated by the bright white spots highlighted by circles, i.e., with longer exposure to the Mg²⁺. FIGS. 22B, 22C, and 22D illustrate the result of bead loading after 30 minutes. 60) minutes, or 120 minutes, respectively, of incubation of the beads with TTM buffer. FIG. 22B shows slightly increased bead aggregation as compared with FIG. 22A. FIGS. 22C and 22D both show significantly increased bead aggregation as compared with FIG. 22A. Therefore, this example shows that prolonged exposure to cations (e.g., in solution) encourages bead aggregation. One potential method to reduce bead aggregation is to minimize the exposure of the beads to cations (e.g., Mg²⁺ in TTM buffer) before dispensing the beads onto the substrate. (e.g., as described in Example 8).

### Example 8: Incubating the Substrate, Instead of the Beads, with Mg²⁺ Promotes High Occupancy of Beads without Aggregation

As described herein, incubating a substrate with cations, such as divalent cations including Mg²⁺ or Ca²⁺, encourages high occupancy of the substrate with beads and minimizes bead aggregation. Although cations promote bead aggregation when the beads themselves are exposed to the ions, for prolonged periods (e.g., as during incubation with a cation-containing buffer as described in Example 7), the same cations also advantageously facilitate dense packing and immobilization of the beads onto small features (i.e., micrometer level features) of the substrate. Aggregation and dense packing are inherently related effects of loading beads onto a substrate. One goal with bead loading is to minimize aggregation, which degrades the sequencing information obtainable from a loaded substrate, while still permitting dense packing, which increases sequencing efficiency. The cations themselves can also screen the high, negative charges of the beads and reduce bead-bead repulsion that occurs on substrates with small feature sizes. It is therefore desirable to investigate the parameters (e.g., the concentration or the incubation time of the cations) of the cations for the treatment, either separately or together, of the substrate and the beads.

To investigate the efficacy of cations in promoting surface occupancy while still minimizing bead aggregation, wafers with a pitch size of 1.8 μm were incubated (prewet) with a TT (10 mM of Tris. pH=7.0; 0.055% Tergitol) buffer containing various amounts of Mg²⁺ (50 mM, 100 mM, 200 mM, and 300 mM) for 0.5-1 minute. The beads were incubated with the TT buffer that lacked Mg²⁺ for 60 minutes in a sample tube prior to being dispensed onto the wafer. As a control, both the wafer and beads were incubated with a TTM buffer (10 mM of Tris. pH=7.0:0.055% Tergitol: 10 mM MgCl₂) for 0.5-1 and 60 minutes, respectively. Table 8 summarizes the resulting average substrate occupancy percentage on the wafers with the beads in different experiments.

TABLE 8
Coupon wafer occupancy with beads on a coupon
wafter with a pitch size of 1.8 μm

| Coupon wafer incubation | Bead incubation | Average substrate |
|---|---|---|
| buffer | buffer | occupancy (%) |
| TTM buffer | TTM buffer | 94 |
| TT buffer + 50 mM Mg²⁺ | TT buffer | 91 |
| TT buffer + 100 mM Mg²⁺ | TT buffer | 92 |
| TT buffer + 200 mM Mg²⁺ | TT buffer | 92 |
| TT buffer + 300 mM Mg²⁺ | TT buffer | 94 |

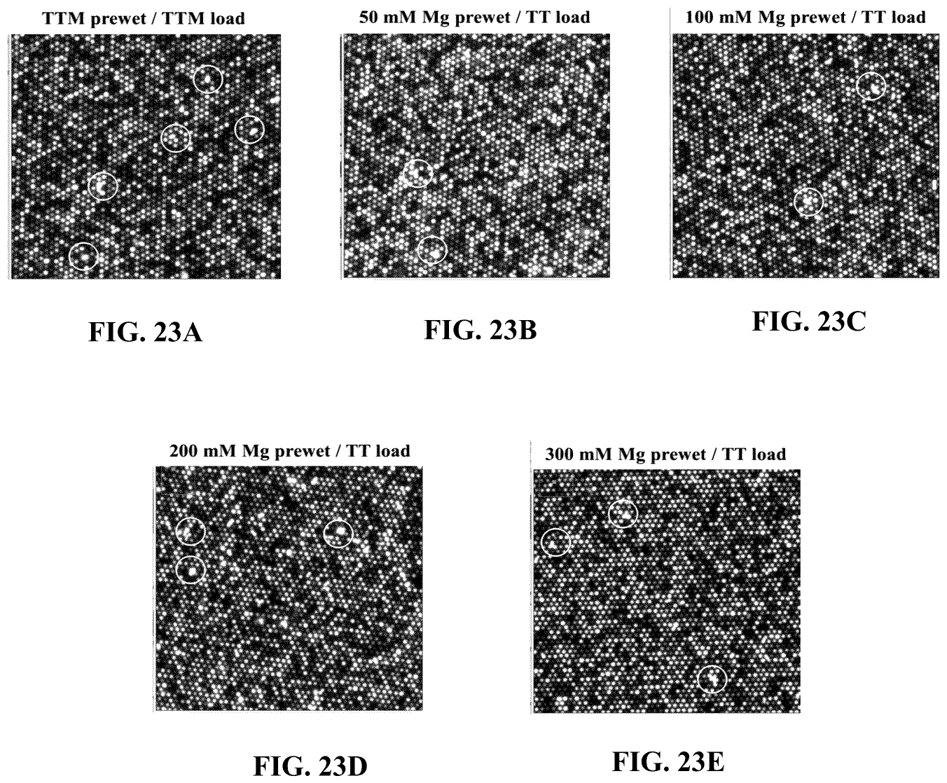

Substrate occupancy measures how much area or how many locations (i.e., micrometer level features) of the substrate are covered with beads. It does not inform on bead aggregation, but rather general success of bead loading. As shown in FIGS. 23A-23E, high average substrate occupancies were achieved in the wafers incubated with various concentrations of Mg²⁺. FIGS. 23B, 23C, 23D, and 23E show the beads incubated with 50 mM Mg²⁺, 100 mM Mg²⁺, 200 mM Mg²⁺, and 300 mM Mg²⁺, respectively. Beads incubated in buffer without any Mg²⁺ (i.e., when the beads were incubated with the TT buffer) showed significantly less bead aggregation on wafer as compared to the control experiment (e.g., FIG. 23A) where the beads and the wafers were both incubated in buffer containing Mg²⁺ (i.e., TTM buffer).

Even when the beads were exposed to the same total amount of Mg²⁺ (e.g., through either incubation of the beads in buffer containing Mg²⁺ or from prewetting a wafer in buffer containing Mg²⁺), incubation in Mg²⁺-containing buffer resulted in aggregates, while Mg²⁺ prewetting discouraged bead aggregation without affecting the substrate occupancy. Thus, the timing (i.e., on or off wafer) of bead exposure to Mg²⁺ is surprisingly important. Table 9 summarizes average substrate occupancy when beads were incubated with or without buffer containing Mg²⁺ (e.g., the TT buffer or the TTM buffer, respectively), while the wafer was incubated with TT buffer containing Mg²⁺ (at either 50 mM or 100 mM) for 0 min. The wafers in this experiment had an average pitch size of 1.5 μm.

TABLE 9
Coupon wafer occupancy with beads on a
coupon wafer with a pitch size of 1.5 μm

| | | Average |
|---|---|---|
| Coupon wafer incubation | Bead incubation | substrate |
| buffer | buffer | occupancy (%) |
| TT buffer + 50 mM Mg²⁺ | TT buffer + 50 mM Mg²⁺ | 88 |
| TT buffer + 100 mM Mg²⁺ | TT buffer | 88 |

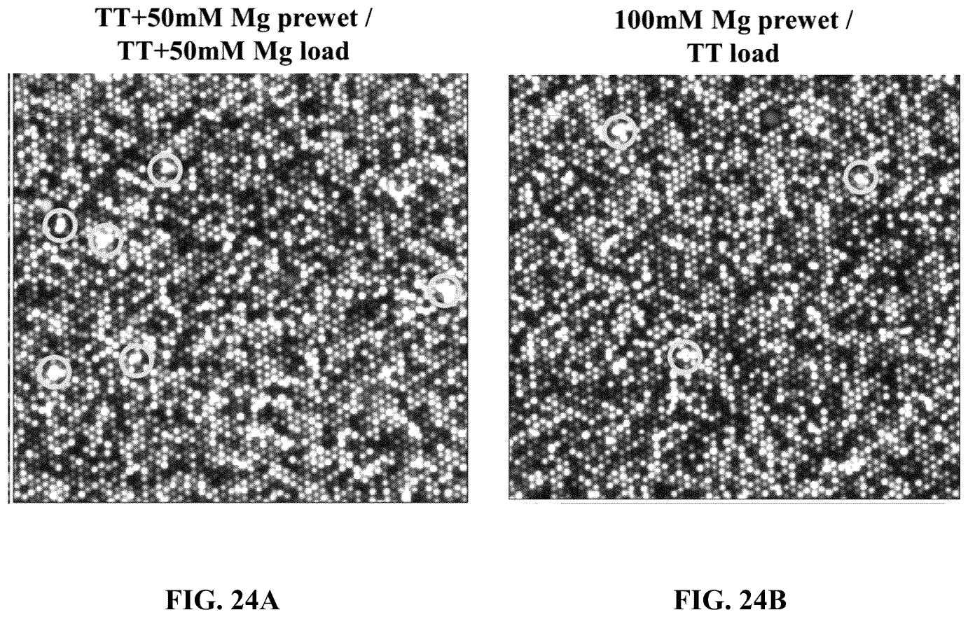

A high occupancy of beads on the substrates was achieved in each scenario. In addition, the beads incubated without any Mg²⁺ (i.e., when the beads were incubated with the TT buffer) (FIG. 24B) showed significantly less bead aggregation compared to that of the control where both the beads and the wafer were incubated with the TTM buffer) (FIG. 24A). This occurred even though the beads were exposed to the same total amount of Mg²⁺ in each experiment.

Therefore, it can be concluded that exposure of beads themselves to cations encourages bead aggregation, but that incubation (prewetting) of the substrate with the same cations has the advantageous result of promoting substrate occupancy without increasing bead aggregation. Hence, one way to reduce the bead aggregation effect is to minimize the exposure of beads to cations before dispensing onto a substrate. Instead, the benefits of dense loading of beads onto wafers can be achieved by incubating the wafers (i.e., alone without the beads, prior to loading) with cations.

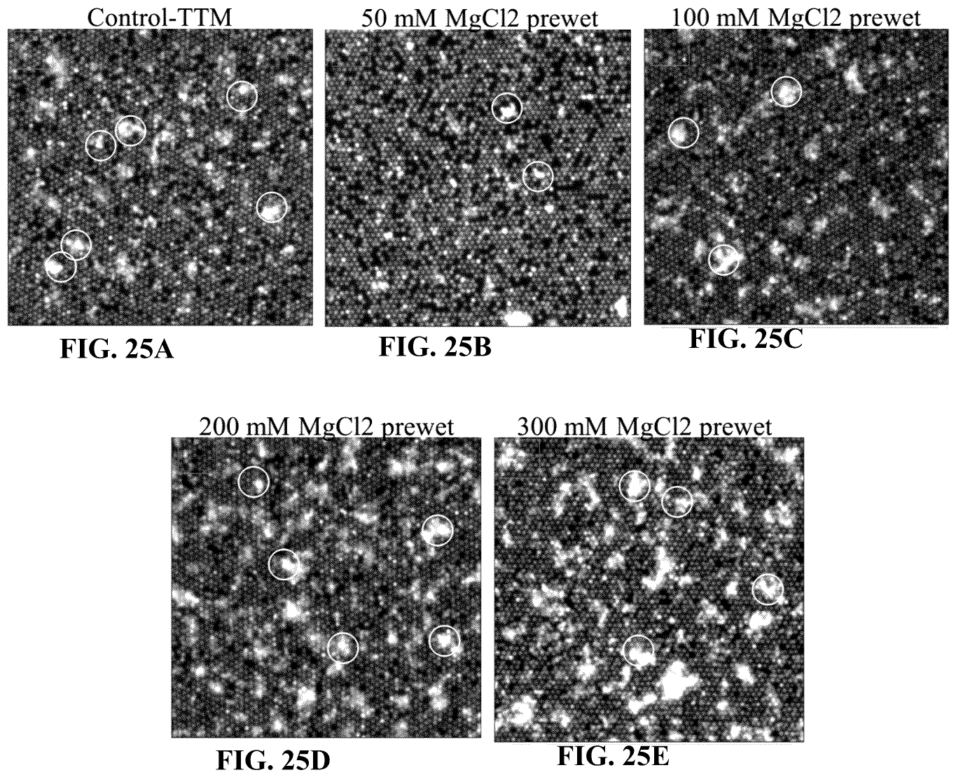

To test whether the same strategy to reduce the bead aggregation while promoting the substrate occupancy would work for other types of beads and substrate layouts, amplified IH beads (B1434 (10%-tBA ATRP in THF)) were prepared by coating the beads with polymer+oligonucleotide conjugate. The beads were attached with DNA amplified by emPCR and fluorescently labeled by hybridizing the amplified DNA strands with a dye-conjugated complementary oligonucleotide probe sequence (i.e., PA39FAM). The labeled beads were incubated in TT buffer (prewet) before loading onto a substrate with a 1.5 μm pitch size. The beads were incubated in 20 μL TT buffer with a concentration of about 20,000,000 beads per μL. The substrate was incubated with the TT buffer with various amounts of Mg²⁺ (e.g., 50 mM (FIG. 25B). 100 mM (FIG. 25C). 200 mM (FIG. 25D), and 300 mM (FIG. 25E)) for 0.5-1 min. As a control (FIG. 25A), the wafer and the beads were both incubated with the TTM buffer for 0.5-1 min and 60 min, respectively. As shown in FIGS. 25A-25E, in all cases when the beads were incubated with the TT buffer, while the wafers were prewet with buffers containing Mg²⁺, high substrate occupancies were observed. The wafer incubated with 50 mM Mg²⁺ showed the least bead aggregation. In contrast, the wafers incubated with either 200 mM or 300 mM Mg²⁺ showed high levels of bead aggregation. Therefore, excess amounts of Mg²⁺ are capable of leading to bead aggregation even when used purely for prewetting wafers.

These experiments demonstrate that Ca²⁺ worked as well as Mg²⁺ in promoting substrate occupancy. However, using Zn²⁺, another divalent cation, significantly increased aggregation of the beads: In one example, the wafers and the beads were incubated (prewet) with a TT (10 mM of Tris. pH=7.0:0.055% Tergitol) buffer containing 10 mM Zn²⁺ (provided in the form of zinc acetate) for 0.5-1 minute and 60 minutes, respectively. After dispensing the beads on the wafers, irreversible and massive bead aggregation was observed by the naked eye. Individual beads could not be resolved under the microscope: only aggregates were observed. In a second example, the wafers were incubated (prewet) with a TT (10 mM of Tris. pH=7.0; 0.055% Tergitol) buffer containing 10 mM Zn²⁺ (provided in the form of zinc acetate) for 0.5-1 minute. The beads were incubated with the TT buffer that lacked Zn²⁺ for 60) minutes in a sample tube prior to being dispensed onto the wafer. Low level of bead aggregation was observed. However, the bead loading was sparse and not dense across coupon surface. Hence, some, but not all divalent cations, can promote the substrate occupancy without inducing bead aggregation.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method for processing a substrate, comprising: (a) providing said substrate comprising a plurality of individually addressable locations, wherein a plurality of particles is immobilized to at least a subset of said plurality of individually addressable locations: (b) adding a buffer solution to said substrate, wherein said buffer solution decreases an average size of at least a subset of said plurality of particles; and (c) subsequent to (b), imaging at least a portion of said substrate.

2. The method of embodiment 1, wherein said imaging detects at least 1000 particles of said plurality of particles. 3. The method of embodiment 1 or 2, wherein said imaging detects at least 10,000 particles of said plurality of particles. 4. The method of any one of embodiments 1-3, wherein said imaging detects at least 100,000 particles of said plurality of particles. 5. The method of any one of embodiments 1-4, wherein said imaging detects at least 1,000,000,000 particles of said plurality of particles. 6. The method of any one of embodiments 1-5, wherein said imaging detects at least 10% of said plurality of particles. 7. The method of any one of embodiments 1-6, wherein said imaging detects at least 50% of said plurality of particles. 8.

The method of any one of embodiments 1-7, wherein said imaging detects at least 70% of said plurality of particles. 9. The method of any one of embodiments 1-8, wherein said imaging detects at least 90% of said plurality of particles. 10. The method of any one of embodiments 1-9, further comprising, subsequent to said imaging of (c), washing said substrate of said buffer solution. 11. The method of embodiment 10, wherein, upon or subsequent to said washing, said average size of said at least said subset of said plurality of particles increases. 12. The method of embodiment 10 or 11, wherein, upon or subsequent to said washing, said average size of said at least said subset of said plurality of particles returns to within 10% of said average size of said plurality of particles in (a). 13. The method of any one of embodiments 10-12, wherein, upon or subsequent to said washing, said average size of said at least said subset of said plurality of particles returns to within 5% of said average size of said plurality of particles in (a). 14. The method of any one of embodiments 10-13, further comprising, subsequent to said washing, (i) performing one or more operations on or with said plurality of particles on said substrate, (ii) repeating (b), and (iii) repeating said imaging. 15. The method of embodiment 14, further comprising repeating a cycle of (i)-(iii) at least 20 times, with washing in between each cycle. 16. The method of embodiment 14 or 15, wherein said one or more operations comprise a nucleotide incorporation reaction with a nucleic acid molecule immobilized to a particle of said plurality of particles. 17. The method of any one of embodiments 1-16, wherein said imaging comprises using photometry. 18. The method of any one of embodiments 1-17, wherein said buffer solution comprises Tris. 19. The method of embodiment 18, wherein said Tris in said buffer solution has a concentration of 20 mM. 20. The method of any one of embodiments 1-19, wherein said buffer solution comprises NaCl. 21. The method of embodiment 20, wherein said NaCl in said buffer solution has a concentration of about 10 μM. 22. The method of embodiment 20 or 21, wherein said NaCl in said buffer solution has a concentration of about 80 pM. 23. The method of any one of embodiments 20-22, wherein said NaCl in said buffer solution has a concentration of about 800 μM. 24. The method of any one of embodiments 20-23, wherein said NaCl in said buffer solution has a concentration of about 8 mM. 25. The method of any one of embodiments 20-24, wherein said NaCl in said buffer solution has a concentration of about 80 mM. 26. The method of any one of embodiments 1-25, wherein said average size, as measured in full-width-half-maximum (FWHM), of said plurality of particles prior to (b) is about 0.1 μm. 27. The method of any one of embodiments 1-26, wherein said average size, as measured in full-width-half-maximum (FWHM), of said plurality of particles prior to (b) is about 1 μm. 28. The method of any one of embodiments 1-27, wherein said decrease of said average size in (b) is at least about 1%, 29. The method of any one of embodiments 1-28, wherein said decrease of said average size in (b) is at least about 10%, 30. The method of any one of embodiments 1-29, wherein said decrease of said average size in (b) is at least about 25%, 31. The method of any one of embodiments 1-30, where said plurality of particles comprises a plurality of beads. 32. The method of embodiment 31, wherein a bead of said plurality of beads comprises a nucleic acid molecule immobilized thereto. 33. The method of embodiment 32, wherein said nucleic acid molecule comprises a fluorescent dye. 34. The method of embodiment 32 or 33, wherein said bead comprises a plurality of nucleic acid molecules, including said nucleic acid molecule, immobilized thereto. 35. The method of embodiment 34, wherein nucleic acid molecules of said plurality of nucleic acid molecules have sequence homology with each other.

36. A method for processing a substrate, comprising: (a) providing said substrate comprising a plurality of individually addressable locations, wherein a plurality of particles is immobilized to at least a subset of said plurality of individually addressable locations; and (b) adding a buffer solution comprising polyethylene glycol (PEG) to said substrate, wherein said buffer solution decreases an average size of at least a subset of said plurality of particles.

37. The method of embodiment 36, wherein said PEG has an average molecular weight of at least 100 daltons. 38. The method of embodiment 36 or 37, wherein said PEG has an average molecular weight of at least 4000 daltons. 39. The method of any one of embodiments 36-38, wherein said PEG has an average molecular weight of at least 8000 daltons. 40. The method of any one of embodiments 36-39, wherein said PEG is at least 0.1%, by weight, in said buffer solution. 41. The method of any one of embodiments 36-40, wherein said PEG is at least 1%, by weight, in said buffer solution. 42. The method of any one of embodiments 36-41, wherein said PEG is at least 5%, by weight, in said buffer solution. 43. The method of any one of embodiments 36-42, wherein said PEG is at least 10%, by weight, in said buffer solution. 44. The method of any one of embodiments 36-43, wherein said buffer solution comprises an ion or a salt derivative thereof. 45. The method of embodiment 44, wherein said ion comprises a cation or a salt derivative thereof. 46. The method of embodiment 45, wherein said cation comprises a divalent cation. 47. The method of embodiment 46, wherein said divalent cation comprises a magnesium ion. 48. The method of embodiment 47, wherein said magnesium ion comprises Mg²⁺. 49. The method of any one of embodiments 44-48, wherein said salt comprises a chloride salt. 50. The method of any one of embodiments 44-49, wherein said ion or said salt derivative in said buffer solution has a concentration of about 1 μM. 51. The method of any one of embodiments 44-50, wherein said ion or said salt derivative in said buffer solution has a concentration of about 50 μM. 52. The method of any one of embodiments 44-51, wherein said ion or said salt derivative in said buffer solution has a concentration of about 500 μM. 53. The method of any one of embodiments 44-52, wherein said ion or said salt derivative in said buffer solution has a concentration of about 5 mM. 54. The method of any one of embodiments 44-53, wherein said ion or said salt derivative in said buffer solution has a concentration of about 50) mM. 55. The method of any one of embodiments 36-54, wherein said buffer solution comprises Tris. 56. The method of embodiment 55, wherein said Tris in said buffer solution has a concentration of 20 mM. 57. The method of any one of embodiments 36-56, wherein said buffer solution comprises NaCl. 58. The method of embodiment 57, wherein said NaCl in said buffer solution has a concentration of about 10 μM. 59. The method of embodiment 57 or 58, wherein said NaCl in said buffer solution has a concentration of about 80 μM. 60. The method of any one of embodiments 57-59, wherein said NaCl in said buffer solution has a concentration of about 800 μM. 61. The method of any one of embodiments 57-60, wherein said NaCl in said buffer solution has a concentration of about 8 mM. 62. The method of any one of embodiments 57-61, wherein said NaCl in said buffer solution has a concentration of about 80 mM. 63. The method of any one of embodiments 36-62, wherein said average size, as measured in full-width-half-maximum (FWHM), of said plurality of particles prior to (b) is about 0.1 μm. 64. The method of any one of embodiments 36-63, wherein said average size, as measured in full-width-half-maximum (FWHM), of said plurality of particles prior to (b) is about 1 μm. 65. The method of any one of embodiments 36-64, wherein said decrease of said average size in (b) is at least about 1%, 66. The method of any one of embodiments 36-65, wherein said decrease of said average size in (b) is at least about 10%, 67. The method of any one of embodiments 36-66, wherein said decrease of said average size in (b) is at least about 25%, 68. The method of any one of embodiments 36-67, where said plurality of particles comprises a plurality of beads. 69. The method of embodiment 68, wherein a bead of said plurality of beads comprises a nucleic acid molecule immobilized thereto. 70. The method of embodiment 69, wherein said nucleic acid molecule comprises a fluorescent dye. 71. The method of embodiment 69 or 70, wherein said bead comprises a plurality of nucleic acid molecules, including said nucleic acid molecule, immobilized thereto. 72. The method of embodiment 71, wherein nucleic acid molecules of said plurality of nucleic acid molecules have sequence homology with each other. 73. The method of any one of embodiments 36-72, further comprising, subsequent to said adding said buffer solution of (b). (c) imaging at least a portion of said substrate. 74. The method of embodiment 73, wherein said imaging detects at least 1000 particles of said plurality of particles. 75. The method of embodiment 73 or 74, wherein said imaging detects at least 10,000 particles of said plurality of particles. 76. The method of any one of embodiments 73-75, wherein said imaging detects at least 100,000 particles of said plurality of particles. 77. The method of any one of embodiments 73-76, wherein said imaging detects at least 1,000,000,000 particles of said plurality of particles. 78. The method of any one of embodiments 73-77, wherein said imaging detects at least 10% of said plurality of particles. 79. The method of any one of embodiments 73-78, wherein said imaging detects at least 50% of said plurality of particles. 80. The method of any one of embodiments 73-79, wherein said imaging detects at least 70% of said plurality of particles. 81. The method of any one of embodiments 73-80, wherein said imaging detects at least 90% of said plurality of particles. 82. The method of any one of embodiments 73-81, further comprising, subsequent to said imaging of (c), washing said substrate of said buffer solution. 83. The method of embodiment 82, wherein, upon or subsequent to said washing, said average size of said at least said subset of said plurality of particles increases. 84. The method of embodiment 82 or 83, wherein, upon or subsequent to said washing, said average size of said at least said subset of said plurality of particles returns to within 10% of said average size of said plurality of particles in (a). 85. The method of any one of embodiments 82-84, wherein, upon or subsequent to said washing, said average size of said at least said subset of said plurality of particles returns to within 5% of said average size of said plurality of particles in (a). 86. The method of any one of embodiments 82-85, further comprising, subsequent to said washing, (i) performing one or more operations on or with said plurality of particles on said substrate, (ii) repeating (b), and (iii) repeating said imaging. 87. The method of embodiment 86, further comprising repeating a cycle of (i)-(iii) at least 20 times, with washing in between each cycle. 88. The method of embodiment 86 or 87, wherein said one or more operations comprise a nucleotide incorporation reaction with a nucleic acid molecule immobilized to a particle of said plurality of particles. 89. The method of any one of embodiments 73-88, wherein said imaging comprises using photometry.

90. A method for processing a substrate, comprising: (a) providing said substrate comprising a plurality of individually addressable locations, wherein a plurality of particles is immobilized to at least a subset of said plurality of individually addressable locations; and (b) adding a buffer solution, across an air gap, to said substrate, wherein said buffer solution decreases an average size of at least a subset of said plurality of particles.

91. The method of embodiment 90, wherein said buffer solution comprises Tris. 92. The method of embodiment 91, wherein said Tris in said buffer solution has a concentration of 20 mM. 93. The method of embodiment 92, wherein said buffer solution comprises NaCl. 94. The method of embodiment 93, wherein said NaCl in said buffer solution has a concentration of about 10 M. 95. The method of embodiment 93 or 94, wherein said NaCl in said buffer solution has a concentration of about 80 pM. 96. The method of any one of embodiments 93-95, wherein said NaCl in said buffer solution has a concentration of about 800 μM. 97. The method of any one of embodiments 93-96, wherein said NaCl in said buffer solution has a concentration of about 8 mM. 98. The method of any one of embodiments 93-97, wherein said NaCl in said buffer solution has a concentration of about 80 mM. 99. The method of any one of embodiments 90-98, wherein said average size, as measured in full-width-half-maximum (FWHM), of said plurality of particles prior to (b) is about 0.1 μm. 100. The method of any one of embodiments 90-99, wherein said average size, as measured in full-width-half-maximum (FWHM), of said plurality of particles prior to (b) is about 1 μm. 101. The method of any one of embodiments 90-100, wherein said decrease of said average size in (b) is at least about 1%, 102. The method of any one of embodiments 90-101, wherein said decrease of said average size in (b) is at least about 10%, 103. The method of any one of embodiments 90-102, wherein said decrease of said average size in (b) is at least about 25%, 104. The method of any one of embodiments 90-103, where said plurality of particles comprises a plurality of beads. 105. The method of embodiment 104, wherein a bead of said plurality of beads comprises a nucleic acid molecule immobilized thereto. 106. The method of embodiment 105, wherein said nucleic acid molecule comprises a fluorescent dye. 107. The method of embodiment 105 or 106, wherein said bead comprises a plurality of nucleic acid molecules, including said nucleic acid molecule, immobilized thereto. 108. The method of embodiment 107, wherein nucleic acid molecules of said plurality of nucleic acid molecules have sequence homology with each other. 109. The method of any one of embodiments 90-108, further comprising, subsequent to adding said buffer solution of (b). (c) imaging at least a portion of said substrate. 110. The method of embodiment 109, wherein said imaging detects at least 1000 particles of said plurality of particles. 111. The method of embodiment 109 or 110, wherein said imaging detects at least 10,000 particles of said plurality of particles. 112. The method of any one of embodiments 109-111, wherein said imaging detects at least 100,000 particles of said plurality of particles. 113. The method of any one of embodiments 109-112, wherein said imaging detects at least 1,000,000,000 particles of said plurality of particles. 114. The method of any one of embodiments 109-113, wherein said imaging detects at least 10% of said plurality of particles. 115. The method of any one of embodiments 109-114, wherein said imaging detects at least 50% of said plurality of particles. 116. The method of any one of embodiments 109-115, wherein said imaging detects at least 70% of said plurality of particles. 117. The method of any one of embodiments 109-116, wherein said imaging detects at least 90% of said plurality of particles. 118. The method of any one of embodiments 109-117, further comprising, subsequent to said imaging of (c), washing said substrate of said buffer solution. 119. The method of embodiment 118, wherein, upon or subsequent to said washing, said average size of said at least said subset of said plurality of particles increases. 120. The method of embodiment 118 or 119, wherein, upon or subsequent to said washing, said average size of said at least said subset of said plurality of particles returns to within 10% of said average size of said plurality of particles in (a). 121. The method of any one of embodiments 118-120, wherein, upon or subsequent to said washing, said average size of said at least said subset of said plurality of particles returns to within 5% of said average size of said plurality of particles in (a). 122. The method of any one of embodiments 118-121, further comprising, subsequent to said washing, (i) performing one or more operations on or with said plurality of particles on said substrate, (ii) repeating (b), and (iii) repeating said imaging. 123. The method of embodiment 122, further comprising repeating a cycle of (i)-(iii) at least 20 times, with washing in between each cycle. 124. The method of embodiment 122 or 123, wherein said one or more operations comprise a nucleotide incorporation reaction with a nucleic acid molecule immobilized to a particle of said plurality of particles. 125. The method of any one of embodiments 109-124, wherein said imaging comprises using photometry.

126. A method for processing a substrate, comprising: (a) providing said substrate comprising a plurality of individually addressable locations, wherein said plurality of individually addressable locations is substantially planar, wherein a plurality of particles is immobilized to at least a subset of said plurality of individually addressable locations; and (b) adding a buffer solution to said substrate, wherein said buffer solution decreases an average size of at least a subset of said plurality of particles.

127. The method of embodiment 126, wherein said buffer solution comprises Tris. 128. The method of embodiment 126, wherein said Tris in said buffer solution has a concentration of 20 mM. 129. The method of any one of embodiments 126-128, wherein said buffer solution comprises NaCl. 130. The method of embodiment 129, wherein said NaCl in said buffer solution has a concentration of about 10 μM. 131. The method of embodiment 129 or 130, wherein said NaCl in said buffer solution has a concentration of about 80 pM. 132. The method of any one of embodiments 130)-132, wherein said NaCl in said buffer solution has a concentration of about 800 μM. 133. The method of any one of embodiments 126-132, wherein said NaCl in said buffer solution has a concentration of about 8 mM. 134. The method of any one of embodiments 126-133, wherein said NaCl in said buffer solution has a concentration of about 80 mM. 135. The method of any one of embodiments 126-134, wherein said average size, as measured in full-width-half-maximum (FWHM), of said plurality of particles prior to (b) is about 0.1 μm. 136. The method of any one of embodiments 126-135, wherein said average size, as measured in full-width-half-maximum (FWHM), of said plurality of particles prior to (b) is about 1 μm. 137. The method of any one of embodiments 126-136, wherein said decrease of said average size in (b) is at least about 1%, 138. The method of any one of embodiments 126-137, wherein said decrease of said average size in (b) is at least about 10%, 139. The method of any one of embodiments 126-138, wherein said decrease of said average size in (b) is at least about 25%, 140. The method of any one of embodiments 126-139, where said plurality of particles comprises a plurality of beads. 141. The method of embodiment 140, wherein a bead of said plurality of beads comprises a nucleic acid molecule immobilized thereto. 142. The method of embodiment 141, wherein said nucleic acid molecule comprises a fluorescent dye. 143. The method of embodiment 141 or 142, wherein said bead comprises a plurality of nucleic acid molecules, including said nucleic acid molecule, immobilized thereto. 144. The method of embodiment 143, wherein nucleic acid molecules of said plurality of nucleic acid molecules have sequence homology with each other. 145. The method of any one of embodiments 126-144, further comprising, subsequent to adding said buffer solution of (b). (c) imaging at least a portion of said substrate. 146. The method of embodiment 145, wherein said imaging detects at least 1000 particles of said plurality of particles. 147. The method of embodiment 145 or 146, wherein said imaging detects at least 10,000 particles of said plurality of particles. 148. The method of any one of embodiments 145-147, wherein said imaging detects at least 100,000 particles of said plurality of particles. 149. The method of any one of embodiments 145-148, wherein said imaging detects at least 1,000,000,000 particles of said plurality of particles. 150. The method of any one of embodiments 145-149, wherein said imaging detects at least 10% of said plurality of particles. 151. The method of any one of embodiments 126-150, wherein said imaging detects at least 50% of said plurality of particles. 152. The method of any one of embodiments 126-151, wherein said imaging detects at least 70% of said plurality of particles. 153. The method of any one of embodiments 126-152, wherein said imaging detects at least 90% of said plurality of particles. 154. The method of any one of embodiments 126-153, further comprising, subsequent to said imaging of (c), washing said substrate of said buffer solution. 155. The method of embodiment 154, wherein, upon or subsequent to said washing, said average size of said at least said subset of said plurality of particles increases. 156. The method of embodiment 154 or 155, wherein, upon or subsequent to said washing, said average size of said at least said subset of said plurality of particles returns to within 10% of said average size of said plurality of particles in (a). 157. The method of any one of embodiments 154-156, wherein, upon or subsequent to said washing, said average size of said at least said subset of said plurality of particles returns to within 5% of said average size of said plurality of particles in (a). 158. The method of any one of embodiments 154-157, further comprising, subsequent to said washing, (i) performing one or more operations on or with said plurality of particles on said substrate, (ii) repeating (b), and (iii) repeating said imaging. 159. The method of embodiment 158, further comprising repeating a cycle of (i)-(iii) at least 20 times, with washing in between each cycle. 160. The method of embodiment 158 or 159, wherein said one or more operations comprise a nucleotide incorporation reaction with a nucleic acid molecule immobilized to a particle of said plurality of particles. 161. The method of any one of embodiments 126-160, wherein said imaging comprises using photometry.

162. A method for processing a substrate, comprising: (a) providing said substrate comprising a plurality of individually addressable locations, wherein said plurality of individually addressable locations are protrusions from a base surface of said substrate, wherein a plurality of particles is immobilized to at least a subset of said plurality of individually addressable locations; and (b) adding a buffer solution to said substrate, wherein said buffer solution decreases an average size of at least a subset of said plurality of particles.

163. The method of embodiment 162, wherein said buffer solution comprises Tris. 164. The method of embodiment 163, wherein said Tris in said buffer solution has a concentration of 20 mM. 165. The method of any one of embodiments 162-164, wherein said buffer solution comprises NaCl. 166. The method of embodiment 165, wherein said NaCl in said buffer solution has a concentration of about 10 μM. 167. The method of embodiment 165 or 166, wherein said NaCl in said buffer solution has a concentration of about 80 μM. 168. The method of any one of embodiments 165-167, wherein said NaCl in said buffer solution has a concentration of about 800 μM. 169. The method of any one of embodiments 165-168, wherein said NaCl in said buffer solution has a concentration of about 8 mM. 170. The method of any one of embodiments 165-169, wherein said NaCl in said buffer solution has a concentration of about 80 mM. 171. The method of any one of embodiments 162-170, wherein said average size, as measured in full-width-half-maximum (FWHM), of said plurality of particles prior to (b) is about 0.1 μm. 172. The method of any one of embodiments 162-171, wherein said average size, as measured in full-width-half-maximum (FWHM), of said plurality of particles prior to (b) is about 1 μm. 173. The method of any one of embodiments 162-172, wherein said decrease of said average size in (b) is at least about 1%, 174. The method of any one of embodiments 162-173, wherein said decrease of said average size in (b) is at least about 10%, 175. The method of any one of embodiments 162-174, wherein said decrease of said average size in (b) is at least about 25%, 176. The method of any one of embodiments 162-175, where said plurality of particles comprises a plurality of beads. 177. The method of embodiment 176, wherein a bead of said plurality of beads comprises a nucleic acid molecule immobilized thereto. 178. The method of embodiment 177, wherein said nucleic acid molecule comprises a fluorescent dye. 179. The method of embodiment 177 or 178, wherein said bead comprises a plurality of nucleic acid molecules, including said nucleic acid molecule, immobilized thereto. 180. The method of embodiment 179, wherein nucleic acid molecules of said plurality of nucleic acid molecules have sequence homology with each other. 181. The method of any one of embodiments 162-180, further comprising, subsequent to adding said buffer solution of (b). (c) imaging at least a portion of said substrate. 182. The method of embodiment 181, wherein said imaging detects at least 1000 particles of said plurality of particles. 183. The method of embodiment 181 or 182, wherein said imaging detects at least 10,000 particles of said plurality of particles. 184. The method of any one of embodiments 181-183, wherein said imaging detects at least 100,000 particles of said plurality of particles. 185. The method of any one of embodiments 181-184, wherein said imaging detects at least 1,000,000,000 particles of said plurality of particles. 186. The method of any one of embodiments 181-185, wherein said imaging detects at least 10% of said plurality of particles. 187. The method of any one of embodiments 181-186, wherein said imaging detects at least 50% of said plurality of particles. 188. The method of any one of embodiments 181-187, wherein said imaging detects at least 70% of said plurality of particles. 189. The method of any one of embodiments 181-188, wherein said imaging detects at least 90% of said plurality of particles. 190. The method of any one of embodiments 181-189, further comprising, subsequent to said imaging of (c), washing said substrate of said buffer solution. 191. The method of embodiment 190, wherein, upon or subsequent to said washing, said average size of said at least said subset of said plurality of particles increases. 192. The method of embodiment 190 or 191, wherein, upon or subsequent to said washing, said average size of said at least said subset of said plurality of particles returns to within 10% of said average size of said plurality of particles in (a). 193. The method of any one of embodiments 190-192, wherein, upon or subsequent to said washing, said average size of said at least said subset of said plurality of particles returns to within 5% of said average size of said plurality of particles in (a). 194. The method of any one of embodiments 190-193, further comprising, subsequent to said washing, (i) performing one or more operations on or with said plurality of particles on said substrate, (ii) repeating (b), and (iii) repeating said imaging. 195. The method of embodiment 194, further comprising repeating a cycle of (i)-(iii) at least 20 times, with washing in between each cycle. 196. The method of embodiment 194 or 195, wherein said one or more operations comprise a nucleotide incorporation reaction with a nucleic acid molecule immobilized to a particle of said plurality of particles. 197. The method of any one of embodiments 162-196, wherein said imaging comprises using photometry.

198. A method for processing a substrate, comprising: (a) providing said substrate comprising a plurality of individually addressable locations, wherein a plurality of particles is electrostatically immobilized to at least a subset of said plurality of individually addressable locations; and (b) adding a buffer solution to said substrate, wherein said buffer solution decreases an average size of at least a subset of said plurality of particles.

199. The method of embodiment 198, wherein said buffer solution comprises Tris. 200. The method of embodiment 199, wherein said Tris in said buffer solution has a concentration of 20 mM. 201. The method of any one of embodiments 198-200, wherein said buffer solution comprises NaCl. 202. The method of embodiment 201, wherein said NaCl in said buffer solution has a concentration of about 10 μM. 203. The method of embodiment 201 or 202, wherein said NaCl in said buffer solution has a concentration of about 80 μM. 204. The method of any one of embodiments 201-203, wherein said NaCl in said buffer solution has a concentration of about 800 μM. 205. The method of any one of embodiments 201-204, wherein said NaCl in said buffer solution has a concentration of about 8 mM. 206. The method of any one of embodiments 201-205, wherein said NaCl in said buffer solution has a concentration of about 80 mM. 207. The method of any one of embodiments 198-206, wherein said average size, as measured in full-width-half-maximum (FWHM), of said plurality of particles prior to (b) is about 0.1 μm. 208. The method of any one of embodiments 198-139, wherein said average size, as measured in full-width-half-maximum (FWHM), of said plurality of particles prior to (b) is about 1 μm. 209. The method of any one of embodiments 198-208, wherein said decrease of said average size in (b) is at least about 1%, 210. The method of any one of embodiments 198-209, wherein said decrease of said average size in (b) is at least about 10%, 211. The method of any one of embodiments 198-210, wherein said decrease of said average size in (b) is at least about 25%, 212. The method of any one of embodiments 198-211, where said plurality of particles comprises a plurality of beads. 213. The method of embodiment 212 wherein a bead of said plurality of beads comprises a nucleic acid molecule immobilized thereto. 214. The method of embodiment 213, wherein said nucleic acid molecule comprises a fluorescent dye. 215. The method of embodiment 213 or 214, wherein said bead comprises a plurality of nucleic acid molecules, including said nucleic acid molecule, immobilized thereto. 216. The method of embodiment 215, wherein nucleic acid molecules of said plurality of nucleic acid molecules have sequence homology with each other. 217. The method of any one of embodiments 198-216, further comprising, subsequent to adding said buffer solution of (b). (c) imaging at least a portion of said substrate. 218. The method of embodiment 217, wherein said imaging detects at least 1000 particles of said plurality of particles. 219. The method of embodiment 217 or 218, wherein said imaging detects at least 10,000 particles of said plurality of particles. 220. The method of any one of embodiments 217-219, wherein said imaging detects at least 100,000 particles of said plurality of particles. 221. The method of any one of embodiments 217-220, wherein said imaging detects at least 1,000,000,000 particles of said plurality of particles. 222. The method of any one of embodiments 217-221, wherein said imaging detects at least 10% of said plurality of particles. 223. The method of any one of embodiments 217-222, wherein said imaging detects at least 50% of said plurality of particles. 224. The method of any one of embodiments 217-223, wherein said imaging detects at least 70% of said plurality of particles. 225. The method of any one of embodiments 217-224, wherein said imaging detects at least 90% of said plurality of particles. 226. The method of any one of embodiments 217-225, further comprising, subsequent to said imaging of (c), washing said substrate of said buffer solution. 227. The method of embodiment 226, wherein, upon or subsequent to said washing, said average size of said at least said subset of said plurality of particles increases. 228. The method of embodiment 226 or 227, wherein, upon or subsequent to said washing, said average size of said at least said subset of said plurality of particles returns to within 10% of said average size of said plurality of particles in (a). 229. The method of any one of embodiments 226-228, wherein, upon or subsequent to said washing, said average size of said at least said subset of said plurality of particles returns to within 5% of said average size of said plurality of particles in (a). 230. The method of any one of embodiments 226-229, further comprising, subsequent to said washing, (i) performing one or more operations on or with said plurality of particles on said substrate, (ii) repeating (b), and (iii) repeating said imaging. 231. The method of embodiment 230, further comprising repeating a cycle of (i)-(iii) at least 20 times, with washing in between each cycle. 232. The method of embodiment 230) or 231, wherein said one or more operations comprise a nucleotide incorporation reaction with a nucleic acid molecule immobilized to a particle of said plurality of particles. 233. The method of any one of embodiments 198-232, wherein said imaging comprises using photometry.

234. A method for processing a substrate, comprising: (a) providing said substrate comprising a plurality of individually addressable locations, wherein a plurality of particles is immobilized to at least a subset of said plurality of individually addressable locations; and (b) adding a buffer solution comprising spermine or a derivative thereof to said substrate, wherein said buffer solution decreases an average size of at least a subset of said plurality of particles.

235. The method of embodiment 234, wherein said spermine or said derivative thereof in said buffer solution has a concentration of about 1 μM. 236. The method of embodiment 234 or 235, wherein said spermine or said derivative thereof in said buffer solution has a concentration of about 50 μM. 237. The method of any one of embodiments 234-236, wherein said spermine or said derivative thereof in said buffer solution has a concentration of about 500 μM. 238. The method of any one of embodiments 234-237, wherein said spermine or said derivative thereof in said buffer solution has a concentration of about 5 mM. 239. The method of any one of embodiments 234-238, wherein said spermine or said derivative thereof in said buffer solution has a concentration of about 10 mM. 240. The method of any one of embodiments 234-239, wherein said buffer solution comprises Tris. 241. The method of embodiment 240, wherein said Tris in said buffer solution has a concentration of 20 mM. 242. The method of any one of embodiments 234-241, wherein said buffer solution comprises NaCl. 243. The method of embodiment 242, wherein said NaCl in said buffer solution has a concentration of about 10 μM. 244. The method of embodiment 242 or 243, wherein said NaCl in said buffer solution has a concentration of about 80 μM. 245. The method of any one of embodiments 242-244, wherein said NaCl in said buffer solution has a concentration of about 800 μM. 246. The method of any one of embodiments 242-245, wherein said NaCl in said buffer solution has a concentration of about 8 mM. 247. The method of any one of embodiments 242-246, wherein said NaCl in said buffer solution has a concentration of about 80 mM. 248. The method of any one of embodiments 242-247, wherein said average size, as measured in full-width-half-maximum (FWHM), of said plurality of particles prior to (b) is about 0.1 μm. 249. The method of any one of embodiments 234-248, wherein said average size, as measured in full-width-half-maximum (FWHM), of said plurality of particles prior to (b) is about 1 μm. 250. The method of any one of embodiments 234-249, wherein said decrease of said average size in (b) is at least about 1%, 251. The method of any one of embodiments 234-250, wherein said decrease of said average size in (b) is at least about 10%, 252. The method of any one of embodiments 234-251, wherein said decrease of said average size in (b) is at least about 25%, 253. The method of any one of embodiments 234-252, where said plurality of particles comprises a plurality of beads. 254. The method of embodiment 253, wherein a bead of said plurality of beads comprises a nucleic acid molecule immobilized thereto. 255. The method of embodiment 254, wherein said nucleic acid molecule comprises a fluorescent dye. 256. The method of embodiment 254 or 255, wherein said bead comprises a plurality of nucleic acid molecules, including said nucleic acid molecule, immobilized thereto. 257. The method of embodiment 256, wherein nucleic acid molecules of said plurality of nucleic acid molecules have sequence homology with each other. 258. The method of any one of embodiments 234-257, further comprising, subsequent to adding said buffer solution of (b). (c) imaging at least a portion of said substrate. 259. The method of embodiment 258, wherein said imaging detects at least 1000 particles of said plurality of particles. 260. The method of embodiment 258 or 259, wherein said imaging detects at least 10,000 particles of said plurality of particles. 261. The method of any one of embodiments 258-260, wherein said imaging detects at least 100,000 particles of said plurality of particles. 262. The method of any one of embodiments 234-261, wherein said imaging detects at least 1,000,000,000 particles of said plurality of particles. 263. The method of any one of embodiments 234-262, wherein said imaging detects at least 10% of said plurality of particles. 264. The method of any one of embodiments 234-263, wherein said imaging detects at least 50% of said plurality of particles. 265. The method of any one of embodiments 234-264, wherein said imaging detects at least 70% of said plurality of particles. 266. The method of any one of embodiments 234-265, wherein said imaging detects at least 90% of said plurality of particles. 267. The method of any one of embodiments 234-266, further comprising, subsequent to said imaging of (c), washing said substrate of said buffer solution. 268. The method of embodiment 267, wherein, upon or subsequent to said washing, said average size of said at least said subset of said plurality of particles increases. 269. The method of embodiment 267 or 268, wherein, upon or subsequent to said washing, said average size of said at least said subset of said plurality of particles returns to within 10% of said average size of said plurality of particles in (a). 270. The method of any one of embodiments 267-269, wherein, upon or subsequent to said washing, said average size of said at least said subset of said plurality of particles returns to within 5% of said average size of said plurality of particles in (a). 271. The method of any one of embodiments 267-270, further comprising, subsequent to said washing, (i) performing one or more operations on or with said plurality of particles on said substrate, (ii) repeating (b), and (iii) repeating said imaging. 272. The method of embodiment 271, further comprising repeating a cycle of (i)-(iii) at least 20 times, with washing in between each cycle. 273. The method of embodiment 271 or 272, wherein said one or more operations comprise a nucleotide incorporation reaction with a nucleic acid molecule immobilized to a particle of said plurality of particles. 274. The method of any one of embodiments 234-273, wherein said imaging comprises using photometry.

275. A method for processing a substrate, comprising: (a) providing said substrate comprising a plurality of individually addressable locations, wherein a plurality of particles is immobilized to at least a subset of said plurality of individually addressable locations: (b) adding a buffer solution to said substrate, wherein said buffer solution decreases an average size of at least a subset of said plurality of particles; and (c) imaging at least a portion of said substrate, wherein said buffer solution is present on said substrate during said imaging.

276. The method of embodiment 275, wherein said buffer solution comprises Tris. 277. The method of embodiment 276, wherein said Tris in said buffer solution has a concentration of 20) mM. 278. The method of any one of embodiments 275-277, wherein said buffer solution comprises NaCl. 279. The method of embodiment 278, wherein said NaCl in said buffer solution has a concentration of about 10 μM. 280. The method of embodiment 278 or 279, wherein said NaCl in said buffer solution has a concentration of about 80 pM. 281. The method of any one of embodiments 278-280, wherein said NaCl in said buffer solution has a concentration of about 800 μM. 282. The method of any one of embodiments 278-281, wherein said NaCl in said buffer solution has a concentration of about 8 mM. 283. The method of any one of embodiments 278-282, wherein said NaCl in said buffer solution has a concentration of about 80 mM. 284. The method of any one of embodiments 275-283, wherein said average size, as measured in full-width-half-maximum (FWHM), of said plurality of particles prior to (b) is about 0.1 μm. 285. The method of any one of embodiments 275-284, wherein said average size, as measured in full-width-half-maximum (FWHM), of said plurality of particles prior to (b) is about 1 μm. 286. The method of any one of embodiments 275-285, wherein said decrease of said average size in (b) is at least about 1%, 287. The method of any one of embodiments 275-286, wherein said decrease of said average size in (b) is at least about 10%, 288. The method of any one of embodiments 275-287, wherein said decrease of said average size in (b) is at least about 25%, 289. The method of any one of embodiments 275-288, where said plurality of particles comprises a plurality of beads. 290. The method of embodiment 289, wherein a bead of said plurality of beads comprises a nucleic acid molecule immobilized thereto. 291. The method of embodiment 290, wherein said nucleic acid molecule comprises a fluorescent dye. 292. The method of embodiment 290) or 291, wherein said bead comprises a plurality of nucleic acid molecules, including said nucleic acid molecule, immobilized thereto. 293. The method of embodiment 292, wherein nucleic acid molecules of said plurality of nucleic acid molecules have sequence homology with each other. 294. The method of any one of embodiments 275-293, further comprising using data from said imaging to detect locations of said plurality of particles on said substrate. 295. The method of embodiment 294, further comprising (i) subsequent to said imaging of (c), washing said substrate of said buffer solution, (ii) subsequent to said washing, performing one or more operations on or with said plurality of particles on said substrate, and (iii) imaging said at least said portion of said substrate. 296. The method of embodiment 295, further comprising repeating a cycle of (ii)-(iii) at least 20 times, with washing in between each cycle, and in absence of contacting said buffer solution to said plurality of particles.

297. A method for processing a substrate, comprising: (a) dispensing a first plurality of particles in a first buffer solution onto the substrate, wherein a subset of the first plurality of particles immobilizes onto a first plurality of individually addressable locations on the substrate; (b) adding a second buffer solution to the substrate, wherein an average maximum dimension of the subset of the first plurality of particles decreases upon contacting the second buffer solution; and (c) dispensing a second plurality of particles in the first buffer solution onto the substrate, wherein a subset of the second plurality of particles immobilizes onto a second plurality of individually addressable locations on the substrate.

298. The method of embodiment 297, wherein subsequent to (c), the method further comprises imaging (d) at least a portion of the substrate. 299. The method of embodiment 298, wherein the imaging comprises using photometry. 300. The method of embodiment 297 or 298, wherein a particle in the subset of the first plurality of particles comprises a nucleic acid molecule immobilized thereto. 301. The method of embodiment 300, wherein the nucleic acid molecule comprises a fluorescent dye. 302. The method of embodiment 300 or 301, wherein the particle comprises a plurality of nucleic acid molecules, including the nucleic acid molecule, immobilized thereto. 303. The method of embodiment 302, wherein nucleic acid molecules of said plurality of nucleic acid molecules have sequence homology with each other. 304. The method of any one of embodiments 297-303, wherein a particle in the subset of the second plurality of particles comprises a nucleic acid immobilized thereto. 305. The method of any one of embodiments 297-304, wherein the adding (b) further comprises making one or more individually addressable locations in the first plurality of individually addressable locations available for immobilizing particles. 306. The method of any one of embodiments 297-305, wherein, prior to the dispensing (c), the second plurality of individually addressable locations do not have a particle of the first plurality of particles immobilized thereto. 307. The method of any one of embodiments 298-306, further comprising, prior to the imaging (d), washing the substrate of the first buffer solution. 308. The method of any one of embodiments 297-307, wherein, upon or subsequent to the dispensing (c), the average maximum dimension of the subset of the first plurality of particles increases. 309. The method of any one of embodiments 297-308, wherein the first plurality of individually addressable locations comprises at least about 100,000 locations. 310. The method of any one of embodiments 297-309, wherein the second plurality of individually addressable locations comprises at least about 100,000 locations. 311. The method of any one of embodiments 298-310, wherein the imaging (d) detects 1000-10,000 particles immobilized to individually addressable locations on the substrate. 312. The method of any one of embodiments 297-311, wherein the average maximum dimension, as measured in full-width half-maximum (FWHM), of the subset of the first plurality of particles prior to the adding (b) is about 0.1 μm. 313. The method of any one of embodiments 297-312, wherein the average maximum dimension, as measured in full-width half-maximum (FWHM), of the subset of the first plurality of particles prior to the adding (b) is about 1 μm. 314. The method of any one of embodiments 297-313, wherein the average maximum dimension of the subset of the first plurality of particles upon the adding (b) decreases by at least about 1%, 315. The method of any one of embodiments 297-314, wherein the average maximum dimension of the subset of the first plurality of particles upon the adding (b) decreases by at least about 10%, 316. The method of any one of embodiments 297-315, wherein the average maximum dimension of the subset of the first plurality of particles upon the adding (b) decreases by at least about 25%, 317. The method of any one of embodiments 297-316, wherein the first plurality of particles comprises a first plurality of beads and the second plurality of particles comprises a second plurality of beads. 318. The method of any one of embodiments 297-317, wherein the adding (b) comprises adding the second buffer solution across an air gap to the substrate. 319. The method of any one of embodiments 297-318, wherein the second buffer solution comprises polyethylene glycol (PEG). 320. The method of embodiment 319, wherein the PEG has an average molecular weight of at least 100 daltons. 321. The method of embodiment 319 or 320, wherein the PEG has an average molecular weight of at least 4000 daltons. 322. The method of any one of embodiments 319-321, wherein the PEG has an average molecular weight of at least 8000 daltons. 323. The method of any one of embodiments 319-322, wherein the second buffer solution comprises at least 0.1% by weight of PEG. 324. The method of any one of embodiments 319-323, wherein the second buffer solution comprises at least 1% by weight of PEG. 325. The method of any one of embodiments 319-324, wherein the second buffer solution comprises at least 5% by weight of PEG. 326. The method of any one of embodiments 319-325, wherein the second buffer solution comprises at least 10% by weight of PEG. 327. The method of any one of embodiments 319-326, wherein the second buffer solution comprises a divalent cation. 328. The method of embodiment 327, wherein the divalent cation comprises a magnesium ion or a calcium ion. 329. The method of any one of embodiments 297-328, wherein: the dispensing (a) comprises incubating the first plurality of particles in the first buffer solution on the substrate for a time period of at least 30 minutes; and the dispensing (b) comprises incubating the second plurality of particles in the second buffer solution on the substrate for a time period of at least 30 minutes. 330. The method of any one of embodiments 297-329, wherein: the dispensing (a) comprises incubating the first plurality of particles in the first buffer solution on the substrate for a time period of at least 60 minutes; and the dispensing (b) comprises incubating the second plurality of particles in the second buffer solution on the substrate for a time period of at least 60 minutes.

331. A method of processing a substrate, comprising: (a) providing a plurality of particles in a first buffer solution; (b) adding an aliquot of a second buffer solution to the plurality of particles in the first buffer solution, thereby providing the plurality of particles in a third buffer solution; (c) incubating the plurality of particles with the third buffer solution; and (d) dispensing the plurality of particles onto the substrate to immobilize at least a subset of the plurality of particles onto a plurality of individually addressable locations on the substrate.

332. The method of embodiment 331, further comprising, subsequent to the dispensing (d), imaging at least a portion of the substrate. 333. The method of embodiment 331, wherein the imaging comprises using photometry. 334. The method of embodiment 332 or 333, further comprising, prior to the imaging (d), washing the substrate of the third buffer solution. 335. The method of any one of embodiments 331-334, wherein a particle in the at least the subset of the plurality of particles comprises a nucleic acid molecule immobilized thereto. 336. The method of embodiment 335, wherein the nucleic acid molecule comprises a fluorescent dye. 337. The method of embodiment 335 or 336, wherein the particle comprises a plurality of nucleic acid molecules, including the nucleic acid molecule, immobilized thereto. 338. The method of embodiment 337, wherein nucleic acid molecules of said plurality of nucleic acid molecules have sequence homology with each other. 339. The method of any one of embodiments 331-338, wherein the second buffer solution comprises at least 1% by weight of PEG. 340. The method of any one of embodiments 331-339, wherein the second buffer solution comprises at least 5% by weight of PEG. 341. The method of any one of embodiments 331-340, wherein the second buffer solution comprises a divalent cation. 342. The method of embodiment 341, wherein the divalent cation is a magnesium ion or a calcium ion. 343. The method of any one of embodiments 331-342, wherein the third buffer solution comprises at least 0.1% by weight of PEG. 344. The method of any one of embodiments 331-343, wherein the third buffer solution comprises at least 1% by weight of PEG. 345. The method of any one of embodiments 331-344, wherein the first buffer solution does not comprise PEG. 346. The method of any one of embodiments 331-345, wherein the first buffer solution does not comprise a divalent cation. 347. The method of any one of embodiments 331-346, wherein the first buffer solution or the second buffer solution comprises a Tris buffer solution. 348. The method of any one of embodiments 331-347, wherein the plurality of individually addressable locations comprises at least about 100,000 locations. 349. The method of any one of embodiments 331-348, wherein an average maximum dimension of the plurality of particles decreases upon or subsequent to the incubating (c). 350. The method of embodiment 349, wherein the average maximum dimension, as measured in full-width half-maximum (FWHM), of the plurality of particles prior to the adding (b) is about 0.1 μm. 351. The method of embodiment 349 or 350, wherein the average dimension, as measured in full-width half-maximum (FWHM), of the subset of the plurality of particles prior to the adding (b) is about 1 μm. 352. The method of any one of embodiments 349-351, wherein the average dimension of the plurality of particles upon or subsequent to the incubating (c) decreases by at least about 1%, 353. The method of any one of embodiments 349-352, wherein the average dimension of the plurality of particles upon or subsequent to the incubating (c) decreases by at least about 10%.

354. The method of any one of embodiments 349-353, wherein the average dimension of the plurality of particles upon or subsequent to the incubating (c) decreases by at least about 25%, 355. The method of any one of embodiments 331-354, wherein the incubating (c) comprises incubating the plurality of particles in the third buffer solution for a time period of at least 10 minutes. 356. The method of any one of embodiments 331-355, wherein the incubating (c) comprises incubating the plurality of particles in the third buffer solution for a time period of at least 1 hour. 357. The method of any one of embodiments 331-356, wherein the plurality of particles comprises a plurality of beads. 358. The method of any one of embodiments 331-357, wherein prior to the adding (b), the plurality of particles is stored in the first buffer solution for a time period of at least 1 hour. 359. The method of any one of embodiments 331-358, wherein prior to the adding (b), the plurality of particles is stored in the first buffer solution for a time period of at least 1 day.

While preferred instances of the present invention have been shown and described herein, it will be obvious to those skilled in the art that such instances are provided by way of example only. It is not intended that the invention be limited by the specific examples provided within the specification. While the invention has been described with reference to the aforementioned specification, the descriptions and illustrations of the instances herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the instances of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-126. (canceled)

127. A method of processing a substrate, comprising:
(a) providing a plurality of particles in a first buffer solution;
(b) adding an aliquot of a second buffer solution to said plurality of particles in said first buffer solution, thereby providing said plurality of particles in a third buffer solution;
(c) incubating said plurality of particles with said third buffer solution; and
(d) dispensing said plurality of particles onto said substrate to immobilize at least a subset of said plurality of particles onto a plurality of individually addressable locations on said substrate.

128. The method of claim 127, further comprising, subsequent to said dispensing in (d), imaging at least a portion of said substrate.

129. The method of claim 128, wherein said imaging comprises using photometry.

130. The method of claim 127, wherein a particle in said at least said subset of said plurality of particles comprises a nucleic acid molecule immobilized thereto.

131. The method of claim 130, wherein said nucleic acid molecule comprises a fluorescent dye.

132. The method of claim 127, wherein said second buffer solution comprises at least 1% or at least 10% by weight of polyethylene glycol (PEG).

133. The method of claim 127, wherein said second buffer solution comprises a divalent cation.

134. The method of claim 133, wherein said divalent cation is a magnesium ion or a calcium ion.

135. The method of claim 127, wherein said third buffer solution comprises at least 0.1% or at least 5% by weight of PEG.

136. The method of claim 127, wherein said first buffer solution does not comprise PEG.

137. The method of claim 127, wherein said first buffer solution does not comprise a divalent cation.

138. (canceled)

139. The method of claim 127, wherein said plurality of individually addressable locations comprises at least about 100,000 locations.

140. The method of claim 127, wherein an average maximum dimension of said plurality of particles decreases upon or subsequent to said incubating in (c).

141. The method of claim 140, wherein said average maximum dimension, as measured in full-width half-maximum (FWHM), of said plurality of particles prior to said adding in (b) is from about 0.1 micrometer (μm) to about 1 μm.

142. The method of claim 140, wherein said average maximum dimension of said plurality of particles upon or subsequent to said incubating in (c) decreases by at least about 1%, at least about 10%, or at least about 25%.

143. The method of claim 127, wherein said incubating in (c) comprises incubating said plurality of particles in said third buffer solution for a time period of at least 10 minutes, or at least 1 hour.

144. The method of claim 127, wherein said plurality of particles comprises a plurality of beads.

145. The method of claim 127, wherein prior to said adding in (b), said plurality of particles is stored in said first buffer solution for a time period of at least 1 hour or at least 1 day.

146. The method of claim 128, wherein said imaging detects at least 100,000 particles of said subset of said plurality of particles.

147. The method of claim 133, wherein said second buffer solution comprises up to about 1 molar (M) of the divalent cation.
